SEQUENCE LISTING (60 sequences)

SEQ ID NO: 1 - DNA, 65 bp, synthetic construct
  misc_feature 1..65: note = Synthetic
  source 1..65: mol_type = other DNA
aaaaatacatattcaatcattaaaacgattgaatggagaacttttgtgtaggctggagctgcttc

SEQ ID NO: 2 - DNA, 65 bp, synthetic construct
  misc_feature 1..65: note = Synthetic
  source 1..65: mol_type = other DNA
ttccccggggcgattttcacctcggggaaattttagttggcgttcttaattctcatgtttgacag

SEQ ID NO: 3 - DNA, 26 bp, synthetic construct
  misc_feature 1..26: note = Synthetic
  source 1..26: mol_type = other DNA
aacggataagacgggcataaatgagg

SEQ ID NO: 4 - DNA, 34 bp, synthetic construct
  misc_feature 1..34: note = Synthetic
  source 1..34: mol_type = other DNA
gtttaataaaaaaaagattaagggatgaaggaac

SEQ ID NO: 5 - DNA, 67 bp, synthetic construct
  misc_feature 1..67: note = Synthetic
  source 1..67: mol_type = other DNA
gctttaatagtgggattaatttccacattaaaacagggattgatcgtgtaggctggagctgcttcgg

SEQ ID NO: 6 - DNA, 74 bp, synthetic construct
  misc_feature 1..74: note = Synthetic
  source 1..74: mol_type = other DNA
cctttaataagcttagcaagagatgttaattttttcagtaagctcttaattctcatgtttgacagggtacccat

SEQ ID NO: 7 - DNA, 24 bp, synthetic construct
  misc_feature 1..24: note = Synthetic
  source 1..24: mol_type = other DNA
gatgtcggagttacgcaggaattg

SEQ ID NO: 8 - DNA, 34 bp, synthetic construct
  misc_feature 1..34: note = Synthetic
  source 1..34: mol_type = other DNA
tatcaaacagtggtttgcttacttcgttattaac

SEQ ID NO: 9 - DNA, 67 bp, synthetic construct
  misc_feature 1..67: note = Synthetic
  source 1..67: mol_type = other DNA
cccatacaattagttaaccaaaaaggggggattttatctccccttgtgtaggctggagctgcttcgg

SEQ ID NO: 10 - DNA, 74 bp, synthetic construct
  misc_feature 1..74: note = Synthetic
  source 1..74: mol_type = other DNA
gcctgccagccctgtttttattagtgcattttgcgcgaggtcacgttaattctcatgtttgacagggtacccat

SEQ ID NO: 11 - DNA, 25 bp, synthetic construct
  misc_feature 1..25: note = Synthetic
  source 1..25: mol_type = other DNA
tctgattcagatcctcaaagagccg

SEQ ID NO: 12 - DNA, 21 bp, synthetic construct
  misc_feature 1..21: note = Synthetic
  source 1..21: mol_type = other DNA
ggcagcacgctctttaacggc

SEQ ID NO: 13 - DNA, 24 bp, synthetic construct
  misc_feature 1..24: note = Synthetic
  source 1..24: mol_type = other DNA
catcagttcgaactgcaatccgtg

SEQ ID NO: 14 - DNA, 20 bp, synthetic construct
  misc_feature 1..20: note = Synthetic
  source 1..20: mol_type = other DNA
catcttcgcaaagcgcgatg

SEQ ID NO: 15 - DNA, 21 bp, synthetic construct
  misc_feature 1..21: note = Synthetic
  source 1..21: mol_type = other DNA
ggcggcagtgaagagaagcct

SEQ ID NO: 16 - DNA, 25 bp, synthetic construct
  misc_feature 1..25: note = Synthetic
  source 1..25: mol_type = other DNA
gcaataacgcgctcgtaatcttctg

SEQ ID NO: 17 - DNA, 21 bp, synthetic construct
  misc_feature 1..21: note = Synthetic
  source 1..21: mol_type = other DNA
cggcgtaccgcaacacttttg

SEQ ID NO: 18 - DNA, 22 bp, synthetic construct
  misc_feature 1..22: note = Synthetic
  source 1..22: mol_type = other DNA
tgtcgcctgcgttaacctggtt

SEQ ID NO: 19 - DNA, 25 bp, synthetic construct
  misc_feature 1..25: note = Synthetic
  source 1..25: mol_type = other DNA
gcgttgttgggatcaattctgagac

SEQ ID NO: 20 - DNA, 21 bp, synthetic construct
  misc_feature 1..21: note = Synthetic
  source 1..21: mol_type = other DNA
tgctgaccaaaaccgacgtcg

SEQ ID NO: 21 - DNA, 24 bp, synthetic construct
  misc_feature 1..24: note = Synthetic
  source 1..24: mol_type = other DNA
gtcgcaacgttgaatgaactgagc

SEQ ID NO: 22 - DNA, 24 bp, synthetic construct
  misc_feature 1..24: note = Synthetic
  source 1..24: mol_type = other DNA
agctcggtactttagcagcagcga

SEQ ID NO: 23 - DNA, 1000 bp, synthetic construct
  misc_feature 1..1000: note = Synthetic
  source 1..1000: mol_type = other DNA
aacggataagacgggcataaatgaggaagaaatggcgcgccctgcaggattcgaacctgcggcccacgacttagaagttcctagaacgacattttaagtcaacaacttaccgcgccatctctgcgctcacacgtcccactacctcaaaacatgtaaagccttgcaagccattgcgaggccttatgtgtctcagttttgtccctcttttttgtactaaaaaacatagtaattgaggataaacctcatgctattttcgcttatatgcctctaaaggcatggcacttaaatagataaaagcaccacaaaagcataaaaaaaccacacagtaaaaccgaaatatgaaacaataacagataattaaaccaaaaacagatagcgcattgtgataatcattcaatactaaacaaaatataaacagtggagcaatatgtaattgactcattaagttagatataaaaaatacatattcaatcattaaaacgattgaatggagaacttttgaacgccaactaaaatttccccgaggtgaaaatcgccccggggaataactagccatttcaatgtaacaattaacccttaaaataaacccagaaggttattaactaaatcacatagaaaaccatcaattatagtatgtataaaataggcgacagcaacccaattacaaattaatggttccagaatatcacatcaaaaaaaacgctgtataatattataattaacatgtagacaacttgtaataaacattatcagtcaattgttttgtttattccatctgtgacgccgattattttctcaaaataatgagatggcgtgacaccataataatctttaaatgcacatatgaaatatgaagtactgttatagccacatttctgggctacgacattgatagaataagagtttgaagttatgagtttttttgcatacctcatcctagtatctctcaatatttcagtaaatgacgttccttcatcccttaatcttttttttattaaac

SEQ ID NO: 24 - DNA, 1000 bp, synthetic construct
  misc_feature 1..1000: note = Synthetic
  source 1..1000: mol_type = other DNA
acgcctttcgccgcgatgtcggagttacgcaggaattgcagcgccacatatttatcattttcgctggcctccgggtcacgcagaatgtcggccacctgttgttggtgcgccggacgcagcatgaacgacgcatcatgaaacgcctggcgcgccaacagagttaacgcttcgggtgcgactttcaaaatctcctgcccttcaaattcagatacgctaacgtgttcgctggttagcaggtaatactcagtatcatcttttttgagtggaaaaggagcctgataatgaaagggtttgtttgacattgttctctcacttactgcctggtttggttatgctctgggcgggtgttccgttgccctgttaaaagcgagtaacaatatcctacacacttttttaacaaaaactgagactagtacgactttttgcggctccaggttacttcccgtaggattcttgctttaatagtgggattaatttccacattaaaacagggattgatcgagcttactgaaaaaattaacatctcttgctaagcttattaaaggcttataacaccttcaggcggccagtccgcctgatttcattttatggataatcattatgaataaatcgctggtagcggtaggcgtcattgttgcgctaggcgtagtctggacaggcggcgcatggtatacaggcaagaagattgaaacccatctcgaagacatggtcgcgcaggcgaacgcgcaactcaaactgacagctcctgaatccaacctggaagtgagttatcaaaactatcatcgcggcgtattcagcagccagttgcaactgttggtgaaacccattgccgggaaagaaaatccgtggattaaaagcggtcagagcgtcatcttcaacgaatcggttgatcatggtcccttcccgcttgcccagcttaaaaaactgaacctgatcccgtcgatggcatcaattcaaaccacgctggttaataacgaagtaagcaaaccactgtttgata

SEQ ID NO: 25 - DNA, 1000 bp, synthetic construct
  misc_feature 1..1000: note = Synthetic
  source 1..1000: mol_type = other DNA
tctgattcagatcctcaaagagccgaaaaacgccctgaccaagcagtatcaggcgctgtttaatctggaaggcgtggatctggaattccgtgacgaggcgctggatgctatcgctaagaaagcgatggcgcgtaaaaccggtgcccgtggcctgcgttccatcgtagaagccgcactgctcgataccatgtacgatctgccgtccatggaagacgtcgaaaaagtggttatcgacgagtcggtaattgatggtcaaagcaaaccgttgctgatttatggcaagccggaagcgcaacaggcatctggtgaataattaaccattcccatacaattagttaaccaaaaaggggggattttatctcccctttaatttttcctctattctcggcgttgaatgtgggggaaacatccccatatactgacgtacatgttaatagatggcgtgaagcacagtcgtgtcatctgattacctggcggaaattaaactaagagagagctcttgacctcgcgcaaaatgcactaataaaaacagggctggcaggctaattcgggcttgccagcctttttttgtctcgctaagttagatggcggatcgggcttgcccttattaaggggtgttgtaaggggatggctggcctgatataactgctgcgcgttcgtaccttgaaggattcaagtgcgatataaattataaagaggaagagaagagtgaataaatctcaattgatcgacaagattgctgcaggggctgatatctctaaagctgcggctggccgtgcgttagatgctattattgcttccgtaactgaatctctgaaagaaggggatgatgtagcactggtaggttttggtacttttgccgttaaagagcgtgctgcccgtactggccgcaacccgcagaccggtaaagagatcaccatcgctgctgctaaagtaccgagcttccgtgcaggtaaagcactgaaagacgcggtaaactaagcgttgtccccagtgggga

SEQ ID NO: 26 - DNA, 500 bp, synthetic construct
  misc_feature 1..500: note = Synthetic
  source 1..500: mol_type = other DNA
tattgaagatccgcttcgatcaggggatgggctactggcgcatcaacttttcatcgcaatggcattactttgatttccgcgatgacgtttctttccagttagtcaaaatggctcaggcctgcaaggaagggaatgtcgccaacagcgaagagagttgggcaacggatgtgctggtggaggtgatcgcctcctgatgatgagccgctcccgatgtggtgtcgggagcggtattttctataaaacttaccgcaatatcaggccggatgcggctgcgccttatccggcccataaccccttacttcctcaaccccgcaaacgcagcccgaatctcttcctccggcagctggatcccgataaacaccatcgtgctatgcggtttttcatcgccccacggcctgtcccagtcggcgctgtagaggcgctggacgccctggaacagcaggcggttaggttcgccgtcaatccacagcatccctttgtaacgtagcagtttatccgcc

SEQ ID NO: 27 - DNA, 1759 bp, Escherichia coli
  source 1..1759: mol_type = genomic DNA
gattcgaactcggcccacgacttagaagttctagaacgacattttaagtcaacaacttaccgcgccatctctgcgctcacacgtcccactacctcaaaacatgtaaagccttgcaagccattgcgaggccttatgtgtctcagttttgtccctcttttttgtactaaaaaacatagtaattgaggataaacctcatgctattttcgcttatatgcctctaaaggcatggcacttaaatagataaaagcaccacaaaagcataaaaaaaccacacagtaaaaccgaaatatgaaacaataacagataattaaaccaaaaacagatagcgcattgtgataatcattcaatactaaacaaaatataaacagtggagcaatatgtaattgactcattaagttagatataaaaaatacatattcaatcattaaaacgattgaatggagaacttttatgcgggcgaaacttctgggaatagtcctgacaacccctattgcgatcagctcttttgcttctaccgagactttatcgtttactcctgacaacataaatgcggacattagtcttggaactctgagcggaaaaacaaaagagcgtgtttatctagccgaagaaggaggccgaaaagtcagtcaactcgactggaaattcaataacgctgcaattattaaaggtgcaattaattgggatttgatgccccagatatctatcggggctgctggctggacaactctcggcagccgaggtggcaatatggtcgatcaggactggatggattccagtaaccccggaacctggacggatgaaagtagacaccctgatacacaactcaattatgccaacgaatttgatctgaatatcaaaggctggctcctcaacgaacccaattaccgcctgggactcatggccggatatcaggaaagccgttatagctttacagccagaggtggttcctatatctacagttctgaggagggattcagagatgatatcggctccttcccgaatggagaaagagcaatcggctacaaacaacgttttaaaatgccctacattggcttgactggaagttatcgttatgaagattttgaactcggtggcacatttaaatacagcggctgggtggaatcatctgataacgatgaacactatgacccgggaaaaagaatcacttatcgcagtaaggtcaaagaccaaaattactattctgttgcagtcaatgcaggttattacgtcacacctaacgcaaaagtttatgttgaaggcgcatggaatcgggttacgaataaaaaaggtaatacttcactttatgatcacaataataacacttcagactacagcaaaaatggagcaggtatagaaaactataacttcatcactactgctggtcttaagtacacattttaagaacgccaactaaaatttccccgaggtgaaaatcgccccgggaataactagccatttcaatgtaacaattaacccttaaaataaacccagaaggttattaactaaatcacatagaaaaccatcaattatagtatgtataaaataggcgacagcaacccaattacaaattaatggttccagaatatcacatcaaaaaaaacgctgtataatattataattaacatgtagacaacttgtaataaacattatcagtcaattgttttgtttattccatctgtgacgccgattattttctcaaaataatgagatggcgtgagaccataataatctttaaatgcacatatgaaatatgaag

SEQ ID NO: 28 - protein, 317 aa, Escherichia coli
MRAKLLGIVLTTPIAISSFASTETLSFTPDNINADISLGTLSGKTKERVYLAEEGGRKVSQLDWKFNNAAIIKGAINWDLMPQISIGAAGWTTLGSRGGNMVDQDWMDSSNPGTWTDESRHPDTQLNYANEFDLNIKGWLLNEPNYRLGLMAGYQESRYSFTARGGSYIYSSEEGFRDDIGSFPNGERAIGYKQRFKMPYIGLTGSYRYEDFELGGTFKYSGWVESSDNDEHYDPGKRITYRSKVKDQNYYSVAVNAGYYVTPNAKVYVEGAWNRVTNKKGNTSLYDHNNNTSDYSKNGAGIENYNFITTAGLKYTF

SEQ ID NO: 29 - DNA, 2812 bp, Escherichia coli
  source 1..2812: mol_type = genomic DNA
gaattccgtgacgaggcgctggatgctatcgctaagaaagcgatggcgcgtaaaaccggtgcccgtggcctgcgttccatcgtagaagccgcactgctcgataccatgtacgatctgccgtccatggaagatgtcgaaaaagtggttatcgacgagtcggtaattgatggtcaaagcaaaccgttgctgatttatggtaagccggaagcgcaacaggcatctggtgaataattaaccattcccatacaattagttaaccaaaaaggggggattttatctcccctttaatttttcctctattctcggcgttgaatgtgggggaaacatccccatatactgacgtacatgttaatagatggcgtgaagcacagtcgtgtcatctgattacctggcggaaattaaactaagagagagctctatgaatcctgagcgttctgaacgcattgaaatccccgtattgccgctgcgcgatgtggtggtttatccgcacatggtcatccccttatttgtcgggcgggaaaaatctatccgttgtctggaagcggcgatggaccatgataaaaaaattatgctggtcgcgcagaaagaagcttcaacggatgagccgggtgtaaacgatcttttcaccgtcgggaccgtggcctctatattgcagatgctgaaactgcctgacggcaccgtcaaagtgctggtcgaggggttacagcgcgcgcgtatttctgcgctctctgacaatggcgaacacttttctgcgaaggcggagtatctggagtcgccgaccattgatgagcgggaacaggaagtgctggtgcgtactgcaatcagccagttcgaaggctacatcaagctgaacaaaaaaatcccaccagaagtgctgacgtcgctgaatagcatcgacgatccggcgcgtctggcggataccattgctgcacatatgccgctgaaactggctgacaaacagtccgttctggagatgtccgacgttaacgaacgtctggaatatctgatggcaatgatggaatcggaaatcgatctgctgcaggttgagaaacgcattcgcaaccgcgttaaaaagcagatggagaaatcccagcgtgagtactatctgaacgagcaaatgaaagctattcagaaagaactcggtgaaatggacgacgcgccggacgaaaacgaagccctgaagcgcaaaatcgacgcggcgaagatgccgaaagaggcaaaagagaaagcggaagcagagttgcagaagctgaaaatgatgtctccgatgtcggcagaagcgaccgtagtgcgtggttatatcgactggatggtacaggtaccgtggaatgcgcgcagcaaggtcaaaaaagacctgcgtcaggcgcaggaaatccttgataccgaccattatggtctggagcgcgtgaaagatcgcatccttgagtaccttgcggttcaaagccgtgtcaacaaaatcaagggaccaatcctctgcctggtagggccgccgggggtaggtaaaacctctcttggtcagtccattgccaaagccaccgggcgtaaatatgtccgtatggcgctgggcggcgtgcgtgatgaagcggaaatccgtggtcaccgccgtacttacatcggttctatgccgggtaaactgatccagaaaatggcgaaagtgggcgtgaaaaacccgctgttcctgctcgatgagatcgacaaaatgtcttctgacatgcgaggcgatccggcctctgcactgcttgaagtgctggatccagagcagaacgtagcgttcagcgaccactacctggaagtggattacgatctcagcgacgtgatgtttgtcgcgacgtcgaactccatgaacattccggcaccgctgctggatcgtatggaagtgattcgcctctccggttataccgaagatgaaaaactgaacatcgccaaacgtcacctgctgccgaagcagattgaacgtaatgcactgaaaaaaggtgagctgaccgtcgacgatagcgccattatcggcattattcgttactacacccgtgaggcgggcgtgcgtggtctggagcgtgaaatctccaaactgtgccgcaaagcggttaagcagttactgctcgataagtcattaaaacatatcgaaattaacggcgataacctgcatgactacctcggtgttcagcgtttcgactatggtcgcgctgataacgaaaaccgtgtcggtcaggtaaccggtctggcgtggacggaagtgggcggtgacttgctgaccattgaaaccgcatgtgttccgggtaaaggcaaactgacctataccggttcgctcggcgaagtgatgcaggagtctattcaggcggcgttaacggtggttcgtgcgcgtgcggaaaaactggggatcaaccctgatttttacgaaaaacgtgacatccacgtccacgtaccggaaggtgcgacgccgaaagatggtccgagtgccggtattgctatgtgcaccgcgctggtttcttgcctgaccggtaacccggttcgtgccgatgtggcaatgaccggtgagatcactctgcgtggtcaggtactgccgatcggtggtttgaaagaaaaactcctggcagcgcatcgcggcgggattaaaacagtgctaattccgttcgaaaataaacgcgatctggaagagattcctgacaacgtaattgccgatctggacattcatcctgtgaagcgcattgaggaagttctgactctggcgctgcaaaatgaaccgtccggcatgcaccattccttgcggcggcggtgctcaacggcctcaacctactactgggctgcttcctaa

SEQ ID NO: 30 - protein, 784 aa, Escherichia coli
MNPERSERIEIPVLPLRDVVVYPHMVIPLFVGREKSIRCLEAAMDHDKKIMLVAQKEASTDEPGVNDLFTVGTVASILQMLKLPDGTVKVLVEGLQRARISALSDNGEHFSAKAEYLESPTIDEREQEVLVRTAISQFEGYIKLNKKIPPEVLTSLNSIDDPARLADTIAAHMPLKLADKQSVLEMSDVNERLEYLMAMMESEIDLLQVEKRIRNRVKKQMEKSQREYYLNEQMKAIQKELGEMDDAPDENEALKRKIDAAKMPKEAKEKAEAELQKLKMMSPMSAEATVVRGYIDWMVQVPWNARSKVKKDLRQAQEILDTDHYGLERVKDRILEYLAVQSRVNKIKGPILCLVGPPGVGKTSLGQSIAKATGRKYVRMALGGVRDEAEIRGHRRTYIGSMPGKLIQKMAKVGVKNPLFLLDEIDKMSSDMRGDPASALLEVLDPEQNVAFSDHYLEVDYDLSDVMFVATSNSMNIPAPLLDRMEVIRLSGYTEDEKLNIAKRHLLPKQIERNALKKGELTVDDSAIIGIIRYYTREAGVRGLEREISKLCRKAVKQLLLDKSLKHIEINGDNLHDYLGVQRFDYGRADNENRVGQVTGLAWTEVGGDLLTIETACVPGKGKLTYTGSLGEVMQESIQAALTVVRARAEKLGINPDFYEKRDIHVHVPEGATPKDGPSAGIAMCTALVSCLTGNPVRADVAMTGEITLRGQVLPIGGLKEKLLAAHRGGIKTVLIPFENKRDLEEIPDNVIADLDIHPVKRIEEVLTLALQNEPSGMQVVTAK

SEQ ID NO: 31 - DNA, 1604 bp, Escherichia coli
  source 1..1604: mol_type = genomic DNA
ccggacgcagcatgaacgacgcatcatgaaacgcctggcgcgccaacagagttaacgcttcgggtgcgactttcaaaatctcctgcccttcaaattcagatacgctaacgtgttcgctggttagcaggtaatactcagtatcatcttttttgagtggaaaaggagcctgataatgaaagggtttgtttgacattgttctctcacttactgcctggtttggttatgctctgggcgggtgttccgttgccctgttaaaagcgagtaacaatatcctacacacttttttaacaaaaactgagactagtacgactttttgcggctccaggttacttcccgtaggattcttgctttaatagtgggattaatttccacattaaaacagggattgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctt

SEQ ID NO: 32 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 33 - DNA, 2877 bp, unidentified
  source 1..2877: mol_type = genomic DNA; note = Bacteriophage T7
tcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacagggctcgcaagtctcgccgtatcagccttgagttcatgcttgagcaagccaataagtttgctaaccataaggccatctggttcccttacaacatggactggcgcggtcgtgtttacgccgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgagaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggctcacggtgttactcgcagtgtgactaagcgttcagtcatgacgctggcttacgggtccaaagagttcggcttccgtcaacaagtgctggaagataccattcagccagctattgattccggcaagggtccgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcc

SEQ ID NO: 34 - protein, 883 aa, unidentified
  source 1..883: note = Bacteriophage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 35 - DNA, 2896 bp, unidentified
  source 1..2896: mol_type = genomic DNA; note = Bacteriophage T3
gatgaggtgcgcattgtggggcaaaccgttacacatagacgcataccttgacaagcgtctacaaggctgatagagtcttttcttacaggtcatcatgaggtggcctgaataggaacgatttattcacaatgaggtaagcaatgaacatcatcgaaaacatcgaaaagaatgacttctcagaaatcgaactggctgctatcccgttcaacacactggctgaccactacggaagcgccttggctaaagagcagttggctttagaacatgagtcttatgagctaggcgagcgccgcttcctcaagatgcttgagcgtcaagcgaaagctggtgagattgcagacaacgcagccgctaagccgttactcgctacgcttctccctaagttaaccacacgtatcgtcgagtggctcgaagagtacgcatcgaagaaaggccgcaagcctagcgcatacgcaccgctccagttactcaagccggaggcctccgcgtttatcaccctgaaagttatccttgcgtcactaaccagtacgaacatgacaaccattcaggccgctgctggtatgctggggaaagccattgaggacgaggcacgatttgggcgcatccgtgacctagaagcgaagcacttcaagaagcacgttgaggaacagcttaacaagcgccacgggcaagtctacaagaaagcatttatgcaggtggtcgaggccgatatgattggtcgaggtctgcttggtggcgaggcgtggtctagctgggataaagaaaccacgatgcacgtagggattcgcctgattgaaatgctgattgaatccacgggtctggtggaattacagcgccacaacgcaggtaacgcaggctctgaccatgaggcactgcaactggcccaagagtacgtggacgtattagcgaagcgtgcaggcgctctggcgggtatctctccgatgttccagccgtgtgtcgtaccgccgaaaccttgggtagcaatcacagggggcggctattgggctaacggtcgcagacctttggcactcgttcgcactcactctaagaagggcttgatgcgctacgaagacgtttacatgccagaagtctacaaggctgtgaacctcgcgcaaaacaccgcatggaaaatcaacaagaaagttcttgctgttgtcaatgagattgttaactggaagaattgcccggtagcagacattccatcgctggagcgccaagagttaccgcctaagcctgacgacattgacaccaacgaggcagcgctcaaggagtggaagaaagccgctgctggtatctatcgcttggacaaggcacgagtgtctcgccgtatcagcttagagttcatgctggagcaggccaacaagttcgcaagtaagaaagcaatctggttcccttacaacatggactggcgcggtcgtgtgtacgctgtgccgatgttcaacccgcaaggcaacgacatgacgaaaggtctgctgacccttgctaaaggcaagccaatcggtgaggaaggtttctactggctgaaaatccacggtgcgaactgtgcgggtgttgataaggttccattcccggagcgcatcgcgttcattgagaagcacgtagacgacattctggcttgcgctaaagacccaatcaataacacttggtgggctgagcaggattcaccgttctgtttcctcgcgttttgcttcgagtatgcaggcgttacgcaccacggtctgagctacaattgctctctgccgctggcgttcgacgggtcttgctctggtatccagcacttctccgcgatgctccgcgatgaggtaggcggtcgtgcggttaacctgctgccaagcgaaaccgtgcaggacatttacggcatcgttgcacagaaagtaaacgagattctcaaacaggatgcaatcaacggcacgcctaacgagatgattaccgtgaccgacaaggacaccggggaaatctcagagaagctcaaacttggaacctcaacgctggcgcaacagtggctggcatatggtgtaacccgtagcgtaactaaacgttcggtcatgacgctggcttacggttccaaggagttcggctttcgtcaacaggtattggatgacaccattcagcctgcaattgacagcggtaagggcttgatgttcacccaaccgaaccaagcggctggctatatggctaagctgatttgggatgcggtaagcgtgaccgtagttgcagcggttgaggcgatgaactggctcaaatctgccgctaagctgctggctgctgaggtcaaggacaagaagaccaaggagattctgcgccaccgttgcgcggttcactggactacgccggacggcttcccggtctggcaggaataccgcaagccactccagaagcgtctcgatatgattttcttagggcaattccgtctgcaaccgacgattaataccctcaaggattcaggcattgacgcacacaagcaggagtctggcatcgctcctaactttgttcactcacaggacggtagccacctccgcatgacagtcgtttatgctcacgagaagtatggcattgagtcctttgcgctcatccatgacagctttgggactatcccggcagacgctggtaagctctttaaggctgtgcgtgaaacgatggttatcacctatgagaacaacgatgtgctggcagacttctactctcagtttgccgaccagctacacgagacccaactggacaagatgcctccgcttccgaagaaaggaaacctgaacctgcaagacattctcaagtctgactttgcctttgcataacaagcacttagcattaaccctcactaacgggagactacttaaggtctcccactttaagacactttaggtactaagagattaaatttatgattaacattaag

SEQ ID NO: 36 - protein, 884 aa, unidentified
  source 1..884: note = Bacteriophage T3
MNIIENIEKNDFSEIELAAIPFNTLADHYGSALAKEQLALEHESYELGERRFLKMLERQAKAGEIADNAAAKPLLATLLPKLTTRIVEWLEEYASKKGRKPSAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 37 - DNA, 2759 bp, unidentified
  source 1..2759: mol_type = genomic DNA; note = Bacteriophage SP6
gcgctcaattaagttttctagtaccgcatgaggatacaagatgcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagtgcgctggatggttgatacaggtatcgaagtacctgagcaaggggagttcgaccttaacgaaatcatggattctgaatacgtatttgcctaatagaacaataaatatacaggtcagccttcgggctggccttttcttttaactattacctgtaacatttaattaacaagtccaacgtgttggacac

SEQ ID NO: 38 - protein, 874 aa, unidentified
  source 1..874: note = Bacteriophage SP6
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 39 - DNA, 2843 bp, unidentified
  source 1..2843: mol_type = genomic DNA; note = Bacteriophage K11
acgaggtgtatcttaggtgtctccgaacggtgaggcacccatagataaactttatccacaaagaggcacacaatgaacgcattaaacattggacgtaatgacttctccgagattgaacttgctgctattccgtacaacatcctgagcgagcactacggggaccaagcggcacgtgagcagttagcactggagcatgaggcatacgagcttggcagacaacgtttcctgaagatgttagaacgtcaggtgaaagctggtgagttcgctgacaacgcggccgctaagccgctggtcttaacgttgcacccacagctgaccaagcgcattgacgactggaaggaggagcaagcaaacgctcgcggtaagaagcctcgcgcatactacccgattaagcacggcgtcgcctcagagttagctgttagcatgggcgctgaggtgctaaaagagaagcgcggagtgtccagtgaggcaatcgcactgctgaccattaaggtcgtcttggggaacgctcacagaccgctcaaaggccacaatccagcagtatcctctcagttaggcaaggctcttgaggatgaggcccgcttcggtcgtatccgtgagcaggaagccgcatacttcaagaagaacgtagcggaccagctggacaagcgagtaggccacgtgtacaagaaggctttcatgcaggtagtcgaggccgatatgatatccaaagggatgctgggcggcgacaactgggcgagctggaaaactgacgagcagatgcacgtagggaccaagctgctggagctactcattgagggaactggtctggtggaaatgaccaagaacaagatggccgatggctccgatgatgtaaccagtatgcagatggtccagctggctccggcctttgtggaactcctgagcaaacgggcaggcgcactcgcgggtatcagcccgatgcaccagccgtgcgtagtccctccgaaaccttgggtggagaccgtaggcggtggctactggtcagtcggtcgccgtccgctggcactggtgcgtacccactccaagaaggcgctgcgccgctacgctgacgtgcacatgccagaggtatacaaggcggtaaacctcgcgcaaaacacgccgtggaaggtgaacaagaaggtgctggcggtagtcaacgagattgtcaactggaagcactgcccggtaggtgacgtcccagcgattgaacgcgaagagttaccgccgcgcccggacgatattgacaccaacgaggtggcacgtaaggcatggcgcaaggaggccgcagcggtctaccgtaaggacaaggcccgccagtctcgccgttgtcgatgtgagttcatggtcgcacaggctaacaagttcgctaaccacaaggccatttggttcccgtacaacatggactggcgcgggcgtgtgtacgctgtgagcatgttcaacccacagggtaacgatatgaccaaggggagtctgacgctggccaagggtaagccaattggtctcgacgggttctactggctgaagattcacggcgcaaactgtgcaggtgtcgacaaggttcccttccctgagcgcatcaagttcatcgaagagaacgagggcaacattctggcgagcgcagcggacccgctgaataacacttggtggacccagcaagattcgccgttctgtttcttagcgttctgcttcgagtacgcaggtgttaagcatcacggcctgaattacaactgctcgctgccgctggcgttcgatgggtcctgctctgggattcagcacttcagcgcgatgctccgagattccatcggtggtcgtgcggttaacctgctgccttctgataccgtgcaggatatctacaagattgtggccgacaaggtgaacgaagtgctccaccagcacgccgtcaacgggtctcagaccgtggtcgagcagattgctgacaaagagactggcgagtttcacgagaaggtgactctgggcgagtccgtactggctgcgcagtggttgcaatatggtgtgacccgcaaggtgactaagcgttcggtcatgacgctggcatacggttccaaagagtcgttggtccgccagcaggttcttgaggacaccattcagcctgctattgacaacggcgagggcctgatgtttacgcaccctaaccaagcagctggctacatggctaagctgatttgggacgctgtgaccgtgaccgtagtggccgctgtcgaggcaatgaactggctgaagtctgccgctaagctgctggctgctgaagtcaaggacaagaagaccaaagaggtgctgcgtaagcgctgcgcaatccactgggtaacacccgatggcttcccggtgtggcaggagtaccgcaagcagaaccaagcgcgcctgaagctggtcttcctcgggcaggccaacgtcaagatgacgtataacactgggaaggactccgagattgatgcccacaagcaggaatccggcatcgctcctaactttgttcactcacaggatggcagtcacctgcgcatgactgtagtacacgccaacgaggtctacgggattgactccttcgcactcattcacgactcctctgggaccattccggctgacgctgggaatctctttaaggcagtccgcgagacgatggtcaagacctacgaggacaacgatgtaattgcagacttctacgaccagtttgccgaccagctgcacgagtctcaactggacaagatgcctgcggttccggccaaaggtgacctgaatctgcgcgatatcttggagtctgacttcgcgtttgcgtaaggtctcaggcaattagggcacactatagggaaccttcgaatgaccgaggg

SEQ ID NO: 40 - protein, 906 aa, unidentified
  source 1..906: note = Bacteriophage K11
MNALNIGRNDFSEIELAAIPYNILSEHYGDQAAREQLALEHEAYELGRQRFLKMLERQVKAGEFADNAAAKPLVLTLHPQLTKRIDDWKEEQANARGKKPRAYYPIKHGVASELAVSMGAEVLKEKRGVSSEAIALLTIKVVLGNAHRPLKGHNPAVSSQLGKALEDEARFGRIREQEAAYFKKNVADQLDKRVGHVYKKAFMQVVEADMISKGMLGGDNWASWKTDEQMHVGTKLLELLIEGTGLVEMTKNKMADGSDDVTSMQMVQLAPAFVELLSKRAGALAGISPMHQPCVVPPKPWVETVGGGYWSVGRRPLALVRTHSKKALRRYADVHMPEVYKAVNLAQNTPWKVNKKVLAVVNEIVNWKHCPVGDVPAIEREELPPRPDDIDTNEVARKAWRKEAAAVYRKDKARQSRRCRCEFMVAQANKFANHKAIWFPYNMDWRGRVYAVSMFNPQGNDMTKGSLTLAKGKPIGLDGFYWLKIHGANCAGVDKVPFPERIKFIEENEGNILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHAVNGSQTVVEQIADKETGEFHEKVTLGESVLAAQWLQYGVTRKVTKRSVMTLAYGSKESLVRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQARLKLVFLGQANVKMTYNTGKDSEIDAHKQESGIAPNFVHSQDGSHLRMTVVHANEVYGIDSFALIHDSSGTIPADAGNLFKAVRETMVKTYEDNDVIADFYDQFADQLHESQLDKMPAVPAKGDLNLRDILESDFAFA

SEQ ID NO: 41 - protein, 884 aa, synthetic construct
  REGION 1..884: note = Synthetic
MNTINIAKNDFSDIELAAIPFNTLADHYGERLAREQLALEHESYEMAEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFAG

SEQ ID NO: 42 - protein, 884 aa, synthetic construct
  REGION 1..884: note = Synthetic
MNTINIAKNDFSDIELAAIPFNTLADHYGERLAREQLALEHESYEMAEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFAG

SEQ ID NO: 43 - protein, 883 aa, synthetic construct
  REGION 1..883: note = Synthetic
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 44 - protein, 235 aa, synthetic construct
  REGION 1..235: note = Synthetic
MYRYLSIAAVVLSAAFSGPALAEGINSFSQAKAAAVKVHADAPGTFYCGCKINWQGKKGVVDLQSCGYQVRKNENRASRVEWEHVVPAWQFGHQRQCWQDGGRKNCAKDPVYRKMESDMHNLQPSVGEVNGDRGNFMYSQWNGGEGQYGQCAMKVDFKEKAAEPPARARGAIARTYFYMRDQYNLTLSRQQTQLFNAWNKMYPVTDWECERDERIAKVQGNHNPYVQRACQARKS

SEQ ID NO: 45 - protein, 353 aa, synthetic construct
  REGION 1..353: note = Synthetic
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 46 - protein, 304 aa, synthetic construct
  REGION 1..304: note = Synthetic
MTVPTYDKFIEPVLRYLATKPEGAAARDVHEAAADALGLDDSQRAKVITSGQLVYKNRAGWAHDRLKRAGLSQSLSRGKWCLTPAGFDWVASHPQPMTEQETNHLAFAFVNVKLKSRPDAVDLDPKADSPDHEELAKSSPDDRLDQALKELRDAVADEVLENLLQVSPSRFEVIVLDVLHRLGYGGHRDDLQRVGGTGDGGIDGVISLDKLGLEKVYVQAKRWQNTVGRPELQAFYGALAGQKAKRGVFITTSGFTSQARDFAQSVEGMVLVDGERLVHLMIENEVGVSSRLLKVPKLDMDYFE

SEQ ID NO: 47 - DNA, 11152 bp, synthetic construct
  misc_feature 1..11152: note = Synthetic
  source 1..11152: mol_type = other DNA
agagttgggcaacggatgtgctggtggaggtgatcgcctcctgatgatgagccgctcccgatgtggtgtcgggagcggtattttctataaaacttaccgctcactcaaaatagtccatatccagtttcggcaccttcaacaaacgtgaagaaacccctacttcgttttcgatcattaagtgcaccaggcgttccccatcaaccaacaccataccctcgacggattgggcaaagtcacgcgcctgagaagtaaatccagaagtggtaataaacaccccacgtttcgctttttgcccagccagtgcgccgtaaaatgcctgtaattctggcctgcctacagtattctgccaacgttttgcctgaacataaactttctccaggccaagtttatcaagcgatatcacaccatcgatgccaccatctccagtaccgccaacacgctgcaaatcatcacggtggccgccataccccaggcgatgcaaaacatccagaacaatgacttcaaagcgcgaaggagaaacctgcaataagttttccagaacctcatcagccaccgcatcacgaagctcttttagcgcctgatctaaccgatcgtccgggctgctctttgcaagttcttcatgatcgggagagtcggctttcggatctaaatcgacggcatccggccgtgacttaagtttgacattcacaaaagcgaaggccagatggttcgtctcctgctccgtcattggctggggatgagacgcaacccagtcaaaacccgcaggagtcaggcaccatttgccacgcgacaaactttgcgacaacccggcacgttttaaacggtcatgcgcccagcctgcacgatttttataaacaagttgtccgctggtaatgactttcgctcgctggctgtcatccagtcctaatgcatccgcggcagcctcatgaacatcacgcgcggctgcaccttccggttttgttgccagataacgcagaacaggttcaataaatttgtcataggtaggaaccgtcatagtacatccttgcagaatcaggtagatgtttttcggctactatagcactacaaaaatagacgaacacgttagaaatgagtcagttgctgtgaccgtggtcattgcccggaaaggtacagaaagctaagatgagatgttatgggccttaaatatttggacaggcccgcacagcaatggattaataacaatgatgaataaatccaattttgaattcctgaagggcgtcaacgacttcacttatgccatcgcctgtgcggcggaaaataactacccggatgatcccaacacgacgctgattaaaatgcgtatgtttggcgaagccacagcgaaacatcttggtctgttactcaacatccccccttgtgagaatcaacacgatctcctgcgtgaactcggcaaaatcgcctttgttgatgacaacatcctctctgtatttcacaaattacgccgcattggtaaccaggcggtgcacgaatatcataacgatctcaacgatgcccagatgtgcctgcgactcgggttccgcctggctgtctggtactaccgtctggtcactaaagattatgacttcccggtgccggtgtttgtgttgccggaacgtggtgaaaacctctatcaccaggaagtgctgacgctaaaacaacagcttgaacagcaggtgcgagaaaaagcgcagactcaggcagaagtcgaagcgcaacagcagaagctggttgccctgaacggctatatcgccattctggaaggcaaacagcaggaaaccgaagcgcaaacccaggctcgccttgcggcactggaagcacagctcgccgagaagaacgcggaactggcaaaacagaccgaacaggaacgtaaggcttaccacaaagaaattaccgatcaggccatcaagcgcacactcaaccttagcgaagaagagagtcgcttcctgattgatgcgcaactgcgtaaagcaggctggcaggccgacagcaaaaccctgcgcttctccaaaggcgcacgtccggaacccggcgtcaataaagccattgccgaatggccgaccggaaaagatgaaacgggtaatcagggctttgcggattatgtgctgtttgtcggcctcaaacccatcgcggtggtagaggcgaaacgtaacaatatcgacgttcccgccaggctcaatgagtcgtatcgctacagtaaatgtttcgataatggcttcctgcgggaaaccttgcttgagcactactcaccggatgaagtgcatgaagcagtgccagagtatgaaaccagctggcaggacaccagcggcaaacaacggtttaaaatccccttctgctactcgaccaacgggcgcgaataccgcgcaacaatgaagaccaaaagcggcatctggtatcgcgacgtgcgtgatacccgcaatatgtcgaaagccttacccgagtggcaccgcccggaagagctgctggaaatgctcggcagcgaaccgcaaaaacagaatcagtggtttgccgataaccctggcatgagcgagctgggcctgcgttattatcaggaagatgccgtccgcgcggttgaaaaggcaatcgtcaaggggcaacaagagatcctgctggcgatggcgaccggtaccggtaaaacccgtacggcaatcgccatgatgttccgcctgatccagtcccagcgttttaaacgcattctcttccttgtcgaccgccgttctcttggcgaacaggcgctgggcgcgtttgaagatacgcgtattaacggcgacaccttcaacagcattttcgacattaaagggctgacggataaattcccggaagacagcaccaaaattcacgttgccaccgtacagtcgctggtgaaacgcaccctgcaatcagatgaaccgatgccggtggcccgttacgactgtatcgtcgttgacgaagcgcatcgcggctatattctcgataaagagcagaccgaaggcgaactgcagttccgcagccagctggattacgtctctgcctaccgtcgcattctcgatcacttcgatgcggtaaaaatcgctctcaccgccaccccggcgctacatactgtgcagattttcggcgagccggtttaccgttatacctaccgtaccgcggttatcgacggttttctgatcgaccaggatccgcctattcagatcatcacccgcaacgcgcaggagggggtttatctctccaaaggcgagcaggtagagcgcatcagcccgcagggagaagtgatcaatgacaccctggaagacgatcaggattttgaagtcgccgactttaaccgtggcctggtgatcccggcgtttaaccgcgccgtctgtaacgaactcaccaattatcttgacccgaccggatcgcaaaaaacgctggtcttctgcgtcaccaatgcccatgccgatatggtggtggaagagctgcgtgccgcgttcaagaaaaagtatccgcaactggagcacgacgcgatcatcaagatcaccggtgatgccgataaagacgcgcgcaaagtgcagaccatgatcacccgcttcaataaagagcggctgcccaatatcgtggtaaccgtcgacctgctgacgaccggcgtcgatattccgtcgatctgtaatatcgtgttcctgcgtaaagtacgcagccgcattctgtacgaacagatgaaaggccgcgccacgcgcttatgcccggaggtgaataaaaccagctttaagatttttgactgtgtcgatatctacagcacgctggagagcgtcgacaccatgcgtccggtggtggtgcgcccgaaggtggaactgcaaacgctggtcaatgaaattaccgattcagaaacctataaaatcaccgaagcggatggccgcagttttgccgagcacagccatgaacaactggtggcgaagctccagcgtatcatcggtctggccacgtttaaccgtgaccgcagcgaaacgatagataaacaggtgcgtcgtctggatgagctatgccaggacgcggcgggcgtgaactttaacggcttcgcctcgcgcctgcgggaaaaagggccgcactggagcgccgaagtctttaacaaactgcctggctttatcgcccgtctggaaaagctgaaaacggacatcaacaacctgaatgatgcgccgatcttcctcgatatcgacgatgaagtggtgagtgtaaaatcgctgtacggtgattacgacacgccgcaggatttcctcgaagcctttgactcgctggtgcaacgttccccgaacgcgcaaccggcattgcaggcagttattaatcgcccgcgcgatctcacccgtaaagggctggtcgagctacaggagtggtttgaccgccagcactttgaggaatcttccctgcgcaaagcatggaaagagacgcgcaatgaagatatcgccgcccggctgattggtcatattcgccgcgctgcggtgggcgatgcgctgaaaccgtttgaggaacgtgtcgatcacgcgctgacgcgcattaagggcgaaaacgactggagcagcgagcaattaagctggctcgatcgtttagcgcaggcgctgaaagagaaagtggtgctcgacgacgatgtcttcaaaaccggcaacttccaccgtcgcggcgggaaggcgatgctgcaaagaacctttgacgataatctcgataccctgctgggcaaattcagcgattatatctgggacgagctggcctgacacgtatacacttcatccttcaggctgcctctgcgttggctgcgctcgttcaccccggtcacgtacttctgtacgctcccggggattcactcacttgccgccttgatgcaacctgaatgattttgtgtatattaccctcggcaatttcttcttctgcggctcgatgaatttgggccgctgcttaatttacggaactcacaatgaacaataacgatctggtcgcgaagctgtggaagctgtgcgacaacctgcgcgatggcggcgtttcctatcaaaactacgtcaatgaactcgcctcgctgctgtttttgaaaatgtgtaaagagaccggtcaggaagcggaatacctgccggaaggttaccgctgggatgacctgaaatcccgcatcggccaggagcagttgcagttctaccgaaaaatgctcgtgcatttaggcgaagatgacaaaaagctggtacaggcagtttttcataatgttagtaccaccatcaccgagccgaaacaaataaccgcactggtcagcaatatggattcgctggactggtacaacggcgcgcacggtaagtcgcgcgatgacttcggcgatatgtacgaagggctgttgcagaagaacgcgaatgaaaccaagtctggtgcaggccagtacttcaccccgcgtccgctgattaaaaccattattcatctgctgaaaccgcagccgcgtgaagtggtgcaggacccggcggcaggtacggcgggctttttgattgaagccgaccgctatgttaagtcgcaaaccaatgatctggacgaccttgatggcgacacgcaggatttccagatccaccgcgcgtttatcggcctcgaactggtgcccggcacccgtcgtctggcactgatgaactgcctgctgcacgatattgaaggcaacctcgaccacggcggcgcaatccgtctgggcaacactctgggtagcgacggtgaaaacctgccgaaggcgcatattgtcgccactaacccgccgtttggcagcgccgcaggcaccaacattacccgcacctttgttcacccgaccagcaacaaacagttgtgctttatgcagcatattatcgaaacgctgcatcccggcggtcgtgcggcggtggtggtgccggataacgtgctgtttgaaggcggcaaaggcaccgacattcgtcgtgacctgatggataagtgtcatctgcacaccattctgcgtctgccgaccggtattttttacgctcagggcgtgaagaccaacgtgctgttctttaccaaagggacggtggcgaacccgaatcaggataagaactgtaccgatgatgtgtgggtgtatgacctgcgtaccaatatgccgagtttcggcaagcgcacaccgtttaccgacgagcatttgcagccgtttgagcgcgtgtatggcgaagacccgcacggtttaagcccgcgcactgaaggtgaatggagttttaacgccgaagagacggaagttgccgacagcgaagagaacaaaaacaccgaccagcatcttgctaccagccgctggcgcaagttcagccgtgagtggatccgcaccgcaaaatccgattcgctggatatctcctggctgaaagataaagacagtattgatgccgacagcctgccggagccggatgtattagcggcagaagcgatgggcgaactggtacaggcgctgtctgaactggatgcgctgatgcgtgaactgggggcgagcgatgaggccgatttgcagcgtcagttgctggaagaagcgtttggtggggtgaaggaatgagtgcggggaaattgccggaggggtgggttatcgccccagtatctacggtcacaactctaatccgaggagtaacgtataaaaaagagcaggcaataaattatctaaaagatgattatttgcctcttatccgtgcgaacaatattcagaatggcaagtttgatactacggacttggtttttgttcctaaaaatcttgttaaagaaagtcaaaaaatatctcctgaagatattgttattgcaatgtcatcagggagcaaatccgtagttggtaaatccgcacatcagcatctaccatttgaatgtagtttcggcgcattttgcggtgtattacgtcctgaaaaacttatattttctggttttattgctcatttcacaaaatcttctctttatcgaaacaaaatttcatcactttctgctggtgcaaatattaataatattaagccggcaagctttgatttgataaatataccaatcccaccacttgccgaacaaaaaatcatcgctgaaaaactcgatacgctgctggcgcaggtagacagcaccaaagcacgttttgagcaaatcccacaaatcctgaaacgttttcgtcaagcggtattggggggcgcagttaatggaaaattgacagaaaaatggcgtaattttgagccgcaacattctgtatttaagaagttaaattttgaatctatcttaactgaattacgtaatggtctttcatcaaagccaaatgaaagtggtgttggtcatccaatactacgcattagttctgtacgtgctggccatgtagatcaaaacgatattcggtttctagaatgttcagaaagtgaactaaaccgccacaaattacaagatggagatcttttatttactcgctataacggaagtttagaatttgttggtgtttgtgggttattgaaaaaattacaacatcaaaatttgctatatcctgataaacttattcgagctcgattaaccaaagatgctttaccagaatatatcgaaatatttttttcatccccctcagcacgaaatgcaatgatgaactgcgtgaaaacaacttctggtcaaaaaggtatttcaggaaaagatatcaaatcccaagttgttttattacctccagtaaaagaacaagccgaaatcgttcgccgcgtcgagcaactcttcgcctacgccgacaccatagaaaaacaggtcaacaacgccttagcccgcgtcaacaacctgacgcaatccatcctggcaaaagcgttccgtggtgaacttaccgcccagtggcgggccgaaaacccggatttgatcagcggagaaaacagcgccgccgcgttgctggaaaaaatcaaagctgaacgcgcagctagcgggggtaaaaaagcctcacgtaaaaaatcctgaacattattttctggcgcacctttccggtgcgctttttattatttcacgccaatcataacccacataaatatatttaaatcattccagaaattgcccattttattctatttttagctggactttccccatatttactgatgatatatacaggtatttagcgcggtgcggatgtgcgccaacacacccgcatcgctaatcacaatcactattcctggagaatagcagttatgactgacacgcattctattgcacaaccgttcgaagcagaagtctccccggcaaataaccgtcatgtcaccgtcggttatgcgagtcgctacccggattacagccgtattcccgccatcaccctgaaaggtcagtggctggaagccgccggttttgccactggcacggcggtagatgtcaaagtgatggaaggctgtattgtcctcaccgcccaaccacccgccgccgaggagagcgaactgatgcagtcgctacgccaggtgtgcaaactgtcggcgcgtaaacaaaagcaggtgcaggcgtttattggagtgattgccggtaaacagaaagtcgcgtaacagcgtttactcccggttaaccaccgggagccttccactgactcaatagaaactttccccctcagtaaatatttaccagtctgattttgcagtaaaaatctattgtttcagtacgttgcgaaagcgataatagaggcttagcaatgaggaaggcatatcttatggaatctattcaaccctggattgaaaaatttattaagcaagcacagcaacaacgttcgcaatccactaaagattatccaacgtcttaccgtaacctgcgagtaaaattgagtttcggttatggtaattttacgtctattccctggtttgcatttcttggagaaggtcaggaagcttctaacggtatatatcccgttattctctattataaagattttgatgagttggttttggcttatggtataagcgacacgaatgaaccacatgcccaatggcagttctcttcagacatacctaaaacaatcgcagagtattttcaggcaacttcgggtgtatatcctaaaaaatacggacagtcctattacgcctgttcccaaaaagtctcacagggtattgattacacccgatttgcctctatgctggacaacataatcaacgactataaattaatatttaattctggcaagagtgttattccacctatgtcaaaaactgaatcatactgtctggaagatgcgttaaatgatttgtttatccctgaaaccacaatagagacgatactcaaacgattaaccatcaaaaaaaatattatcctccaggggccgcccggcgttggaaaaacctttgttgcacgccgtctggcttacttgctgacaggagaaaaggctccgcaacgcgtcaatatggttcagttccatcaatcttatagctatgaggattttatacagggctatcgtccgaatggcgtcggcttccgacgtaaagacggcatattttacaatttttgtcagcaagctaaagagcagccagagaaaaagtatatttttattatagatgaaatcaatcgtgccaatctcagtaaagtatttggcgaagtgatgatgttaatggaacatgataaacgaggtgaaaactggtctgttcccctaacctactccgaaaacgatgaagaacgattctatgtcccggagaatgtttatatcatcggtttaatgaatactgccgatcgctctctggccgttgttgactatgccctacgcagacgattttctttcatagatattgagccaggttttgatacaccacagttccggaattttttactgaataaaaaagcagaaccttcatttgttgagtctttatgccaaaaaatgaacgagttgaaccaggaaatcagcaaagaggccactatccttgggaaaggattccgcattgggcatagttacttctgctgtgggttggaagatggcacctctccggatacgcaatggcttaatgaaattgtgatgacggatatcgcccctttactcgaagaatatttctttgatgacccctataaacaacagaaatggaccaacaaattattaggggactcatagtggaacagcccgtgatacctgtccgtaatatctattacatgcttacctatgcatggggttatttacaggaaattaagcaggcaaaccttgaagccatacccggtaacaatcttcttgatatcctggggtatgtattaaataaaggggttttacagctttcacgccgagggcttgagcttgattacaatcctaacaccgagatcattcctggcatcaaagggcgaatagagtttgctaaaacaatacgcggcttccatcttaatcatgggaaaaccgtcagtacttttgatatgcttaatgaagacacgctggctaaccgaattataaaaagcacattagccatattaattaagcatgaaaagttaaattcaactatcagagatgaagctcgttcactttatagaaaattaccgggcattagcactcttcatttaactccgcagcatttcagctatctgaatggcggaaaaaatacgcgttattataaattcgttatcagtgtctgcaaattcatcgtcaataattctattccaggtcaaaacaaaggacactaccgtttctatgattttgaaagaaacgaaaaagagatgtcattactttatcaaaagtttctttatgaattttgccgtcgtgaattaacgtctgcaaacacaacccgctcttatttaaaatgggatgcatcgagtatatcggatcagtcacttaatttgttacctcgaatggaaactgacatcaccattcgctcatcagaaaaaatacttatcgttgacgccaaatactataagagcattttttcacgacgaatgggaacagaaaaatttcattcgcaaaatctttatcaactgatgaattacttatggtcgttaaagcctgaaaatggcgaaaacataggggggttattaatatatccccacgtagataccgcagtgaaacatcgttataaaattaatggcttcgatattggcttgtgtaccgtcaatttaggtcaggaatggccgtgtatacatcaagaattactcgacattttcgatgaatatctcaaataaaatatcaggccggatgcggctgcgccttatccggcccataaccccttacttcctcaaccccgcaaacgcagcccgaatctcttcctccggcagctggatc

SEQ ID NO: 48 - DNA, 1262 bp, synthetic construct
  misc_feature 1..1262: note = Synthetic
  source 1..1262: mol_type = other DNA
caatttctacaaaacacttgatactgtatgagcatacagtataattgcttcaacagaacatattgactatccggtattacccggcatgacaggagtaaaaatggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgacaacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcgccgtttaaacaggctgaattccagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcgactgcctggctgaaagataacccggaaaccgcgaaagagatcgagaagaaagtacgtgagttgctgctgagcaacccgaactcaacgccggatttctctgtagatgatagcgaaggcgtagcagaaactaacgaagatttttaatcgtcttgtttgatacacaagggtcgcatctgcggcccttttgcttttttaagttgtaaggatatgccatgacagaatcaacatcccgtcgcccggcata

SEQ ID NO: 49 - DNA, 2573 bp, Escherichia coli
  source 1..2573: mol_type = genomic DNA
ggatccattttcagccttggcacaagggaagagcggaagacgggtgggcgcagagcgatacttcgctactattttcacccagaagtgctccaccacttgcgaaacgcccgactgcgaacgcttctggtgacaacccaggggattcagcccctgtagccgatgatgaacgtggccagccgttcaatcacctcggcgatgcaccccctcaggtgttatcacaggactggctcctccaacaccgttacttggcaacgcgcctcttctggctgcgtagcgcaggtagtacatttataaataaagggtgagcggggcggttgtcaacgatggggtcatgcggatttttcatccactcctggcggtcagtagttcagctaataaatgcttcactgcgctaagggtttacactcaacattacgctaacggcactaaaaccatcacatttttctgtgactggcgctacaatcttccaaagtcacaattctcaaaatcagaagagtattgctaatgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactcgtggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaatcatcgtcgatatgtaggccggataaggcgttcacgccgcatccggcaaccgatgcctgatgcgacgcggtccggtcttatccggcctacaggtcgatgccgatatgtacactgtattcggcaattaagacatagcacgagggaggaaataaccggaagaacaaaaagacgttaggggatttttttatcagataaatccccttgtctgtaatttaacggaaatcataccgtgaggttaatcctaaaatagatttttaatcgttgtttatttcggaaaatacgcagattaattgcttttgtttttattttaagtttatgatttttattgttatttaaatataagttgaaacttatatttgatattcattccaattatcctaaaacgccatcgctaattccccgcgccgtaattcgcatgc

SEQ ID NO: 50 - protein, 549 aa, synthetic construct
  REGION 1..549: note = Synthetic
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 51 - DNA, 1371 bp, Escherichia coli
  source 1..1371: mol_type = genomic DNA
atgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 52 - protein, 456 aa, synthetic construct
  REGION 1..456: note = Synthetic
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 53 - DNA, 1437 bp, Escherichia coli
  source 1..1437: mol_type = genomic DNA
atggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcgggttgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaa

SEQ ID NO: 54 - protein, 478 aa, synthetic construct
  REGION 1..478: note = Synthetic
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 55 - DNA, 1122 bp, Escherichia coli
  source 1..1122: mol_type = genomic DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataa

SEQ ID NO: 56 - protein, 373 aa, synthetic construct [sequence omitted]

SEQ ID NO: 57 - DNA, 966 bp, Escherichia coli
  source 1..966: mol_type = genomic DNA
atgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgacgtggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaa

SEQ ID NO: 58 - protein, 321 aa, synthetic construct
  REGION 1..321: note = Synthetic
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 59 - DNA, 837 bp, Escherichia coli
  source 1..837: mol_type = genomic DNA
gtggacgtaagacagagcatccacagtgcgcacgcaaaaacgctggatacccaagggctgcgcaatgaatttttggttgaaaaggtatttgtcgccgatgagtacaccatggtttacagccacattgaccgaattattgttggcggcattatgccgataactaaaacggtttccgttggcggggaagttggtaaacaactcggcgtaagctatttccttgaacgtcgcgagttaggtgttatcaatattggcggtgccggtacgattactgtcgatggccaatgctatgaaatcggtcaccgcgacgccctgtatgttggtaaaggtgcaaaagaagttgtctttgccagtattgataccggcactccggcgaagttttattacaattgcgcacccgcgcatacgacgtatcccaccaaaaaagtcacaccggacgaagtatctccagtcacgttaggcgataacctcaccagtaaccgtcgcacgattaacaaatattttgtcccggatgtactggaaacctgccaattgagtatggggctgacggagctggctccgggtaacttgtggaacaccatgccgtgtcacacccacgagcgccggatggaagtttatttctatttcaatatggatgatgacgcctgcgttttccacatgatggggcagccgcaagaaacgcgtcatattgtgatgcataacgagcaggcggtgatctccccgagctggtcgatccattccggtgtcggaaccaaagcttatacctttatctggggcatggtcggtgaaaaccaggtctttgatgatatggaccatgtggccgttaaagatttgcgctag

SEQ ID NO: 60 - protein, 278 aa, synthetic construct
  REGION 1..278: note = Synthetic
MDVRQSIHSAHAKTLDTQGLRNEFLVEKVFVADEYTMVYSHIDRIIVGGIMPITKTVSVGGEVGKQLGVSYFLERRELGVINIGGAGTITVDGQCYEIGHRDALYVGKGAKEVVFASIDTGTPAKFYYNCAPAHTTYPTKKVTPDEVSPVTLGDNLTSNRRTINKYFVPDVLETCQLSMGLTELAPGNLWNTMPCHTHERRMEVYFYFNMDDDACVFHMMGQPQETRHIVMHNEQAVISPSWSIHSGVGTKAYTFIWGMVGENQVFDDMDHVAVKDLR

(19) United States
(12) Patent Application Publication
(10) Pub. No.: US 2025/0051736 A1
(43) Pub. Date: Feb. 13, 2025

(54) CUSTOM BACTERIAL STRAIN FOR RECOMBINANT PROTEIN PRODUCTION

(71) Applicant: ModernaTX, Inc., Cambridge, MA (US)

(72) Inventor: Kevin Smith, Cambridge, MA (US)

(73) Assignee: ModernaTX, Inc., Cambridge, MA (US)

(21) Appl. No.: 18/702,280

(22) PCT Filed: Oct. 17, 2022
(86) PCT No.: PCT/US2022/078214

§ 371 Date: Apr. 17, 2024

Related U.S. Application Data
(60) Provisional application No. 63/256,690, filed on Oct. 18, 2021.
(60) Provisional application No. 63/304,195, filed on Jan. 28, 2022.

(51) Int. Cl. C12N 9/12; C12N 15/70

(52) U.S. Cl. CPC C12N 9/1247; C12N 15/70; C12Y 207/07006

(57) ABSTRACT

Described here are genetically modified microorganisms with reduced protease activity for the expression of recombinant proteins and without mucoid phenotypes. Also described are methods of making and using the same.

### RELATED APPLICATIONS

This application claims the benefit under 35 U.S.C. § 119 (c) of the earlier filing dates of U.S. Provisional Application No. 63/256,690, filed Oct. 18, 2021, and U.S. Provisional Application No. 63/304,195, filed Jan. 28, 2022, the contents of which are incorporated by reference herein in their entirety.

### REFERENCE TO AN ELECTRONIC SEQUENCE LISTING

The contents of the electronic sequence listing (M137870169WO00-SEQ-NTJ.xml; Size: 122,328 bytes; and Date of Creation: Oct. 14, 2022) are herein incorporated by reference in their entirety.

### BACKGROUND

Escherichia coli (E. coli) has a long history in biotechnology and drug development, and has been used as a host for plasmid DNA production for many years. This is due to a variety of reasons, among them are genetic simplicity (e.g., smaller number of genes of ˜4,400), growth rate, safety, success in hosting foreign DNA, and ease of care of E. coli. The long history of E. coli use has also made it a well characterized organism which has been manipulated in various ways. For example, several different strains have been constructed for different purposes including cloning, plasmid DNA production, and protein expression. Most commonly, E. coli K12 derivatives used such as DH5α, JM108, DH10β, and others are used for plasmid DNA cloning and production because they possess specific genomic mutations that are desirable for cloning purposes. These primarily result in the inactivation of genes that encode nucleases, recombinases, and other enzymes that reduce DNA stability, purity, and cloning efficiency of the strain.

Due to its history as a host bacterium for plasmid cloning and production, E. coli is also commonly used for the expression of recombinant proteins. However, the E. coli genome encodes multiple proteases, such as OmpT and Lon, that can cleave proteins, especially mutant or foreign proteins, expressed by the bacterium. The presence of these proteases reduces the amount of a recombinant protein that can be successfully purified from a given E. coli cell. Furthermore, some protease-deficient variants of E. coli exhibit a mucoid phenotype, which interferes with genetic manipulation of bacteria, mixing of cells and maintenance of an aerobic environment during fermentation, and separation of bacterial cells from a culture medium.

### SUMMARY

Described herein are engineered bacterial strains and vectors for enhanced plasmid DNA production, recombinant protein expression, and improved protein yield. Bacterial proteases, such as OmpT and Lon, cleave proteins, especially mutant and foreign proteins, which limits the yield of recombinant proteins expressed in bacterial cells. Many bacterial strains commonly used for recombinant protein expression, such as Escherichia coli BL21 cells, are modified to delete the ompT gene, and E. coli B strains, such as BL21, are considered naturally deficient in Lon protease activity due to one or more mutations in the promoter of the lon gene. However, total deletion of the lon gene from the bacterial chromosome further improved protein yield, but resulted in a mucoid phenotype in the bacterial strain, suggesting that the lon gene is still expressed in E. coli B strains. This mucoid phenotype, which was attributed to the accumulation of excess polysaccharides in the absence of Lon protease activity, interferes with efficient pelleting of bacteria and subsequent purification of recombinant protein. This mucoid phenotype was absent in strains lacking manA, which catalyzes the first step in GDP-mannose synthesis and is thus required for production of polysaccharides containing mannose. Therefore, a bacterial strain lacking ompT, lon, and one or more genes required for synthesis of extracellular polysaccharides (e.g., manA), produces greater amounts of a recombinant proteins relative to an unmodified bacterial strain.

Accordingly, some aspects of the present disclosure relate to a genetically modified microorganism comprising a genome in which an ompT gene and a lon gene have been mutated, disabled, or deleted. In some embodiments, the microorganism does not express a functional form of one or more proteins selected from the group consisting of G6PI, ManA, ManB, ManC, Gmd, Fcl, and KduI. In some embodiments, the genome comprises a mutation in a gene selected from the group consisting of pgi, manA, manB, manC, gmd, fcl, and kduI. In some embodiments, the genome comprises a mutation in a promoter operably linked to a gene selected from the group consisting of pgi, manA, manB, manC, gmd, fel, and kduI. In some embodiments, the genome does not comprise a nucleic acid sequence encoding a carbohydrate metabolism protein selected from the group consisting of G6PI, ManA, ManB, ManC, Gmd, Fcl, and KduI.

In some embodiments, the microorganism does not express functional ManA. In some embodiments, the genome comprises a mutation in a manA gene or a promoter operably linked to the manA gene. In some embodiments, the genome does not comprise a nucleic acid sequence encoding ManA.

In some embodiments, the genome comprises i) a first nucleic acid sequence having at least 90% sequence identity to SEQ ID NO: 23, and ii) a second nucleic acid sequence having at least 90% sequence identity to SEQ ID NO: 25. In some embodiments, the genome further comprises a third nucleic acid sequence having at least 90% sequence identity to SEQ ID NO: 24. In some embodiments, the microorganism does not exhibit a mucoid phenotype. In some embodiments, the genetically modified microorganism is not capable of synthesizing mannose. In some embodiments, the genetically modified microorganism is not capable of synthesizing fucose.

In some embodiments, the genome does not comprise a nucleic acid sequence encoding endA, the genome does not comprise a nucleic acid sequence encoding recA, and the EcoK1 restriction system has been inactivated. In some embodiments, the genotype of the microorganism is ΔendA ΔrecA Δ(mrr-hsdRMS-symE-mcrBC) ΔompT ΔmanA Δlon. In some embodiments, the microorganism is E. coli. In some embodiments, the microorganism is derived from E. coli MG1655.

In some embodiments, the genetically modified microorganism comprises a nucleic acid sequence encoding a recombinant protein. In some embodiments, the nucleic acid sequence encoding a recombinant protein is located in the genome of the microorganism. In some embodiments, the nucleic acid sequence encoding a recombinant protein is located on a plasmid. In some embodiments, the recombinant protein is an RNA polymerase. In some embodiments, the RNA polymerase is T7 RNA polymerase, T3 RNA polymerase, SP6 RNA polymerase, or K11 RNA polymerase.

In some embodiments, the RNA polymerase comprises (a) an amino acid substitution at a binding site residue for de novo RNA synthesis; and (b) an amino acid modification that causes increased transcription efficiency, relative to wild-type RNA polymerase. In some embodiments, the amino acid modification is an amino acid substitution at position 47, relative to the wild-type RNA polymerase, wherein the wild-type RNA polymerase comprises the amino acid sequence of SEQ ID NO: 34. In some embodiments, the amino acid substitution at position 47 is G47A. In some embodiments, the amino acid modification comprises an additional C-terminal amino acid, relative to the wild-type RNA polymerase. In some embodiments, the additional C-terminal amino acid is glycine. In some embodiments, the RNA polymerase comprises the amino acid sequence SEQ NO: 41. In some embodiments, the RNA polymerase variant comprises an amino acid substitution at position 350, and/or an amino acid substitution at position 351 relative to the wild-type RNA polymerase, wherein the wild-type RNA polymerase comprises the amino acid sequence of SEQ ID NO: 34. In some embodiments, the amino acid substitution at position 350 is E350W. In some embodiments, the amino acid substitution at position 351 is D351V. In some embodiments, the amino acid substitution at position 350 is E350W. and the amino acid substitution at position 351 is D351V. In some embodiments, the RNA polymerase variant comprises the amino acid sequence of SEQ ID NO: 42. In some embodiments, the RNA polymerase variant comprises the amino acid sequence of SEQ ID NO: 43.

In some aspects, the disclosure relates to a method for producing a polypeptide or protein, comprising the steps of i) introducing a nucleic acid molecule comprising a sequence encoding a polypeptide or protein into one of the genetically modified microorganisms described herein; ii) culturing the genetically modified organism under conditions suitable for expression of the polypeptide or protein; and iii) isolating the polypeptide or protein.

In some aspects, the disclosure relates to a method for producing a polypeptide or protein, comprising the steps of i) culturing one of the genetically modified microorganisms described herein under conditions suitable for expression of the polypeptide or protein; and ii) isolating the polypeptide or protein.

In some embodiments of the methods described herein, the polypeptide or protein is an RNA polymerase. In some embodiments, the RNA polymerase is T7 RNA polymerase. T3 RNA polymerase, SP6 RNA polymerase, or K11 RNA polymerase. In some embodiments, the RNA polymerase comprises (a) an amino acid substitution at a binding site residue for de novo RNA synthesis; and (b) an amino acid modification that causes increased transcription efficiency, relative to wild-type RNA polymerase. In some embodiments, the amino acid modification is an amino acid substitution at position 47, relative to the wild-type RNA polymerase, wherein the wild-type RNA polymerase comprises the amino acid sequence of SEQ ID NO: 34. In some embodiments, the amino acid substitution at position 47 is G47A. In some embodiments, the amino acid modification comprises an additional C-terminal amino acid, relative to the wild-type RNA polymerase. In some embodiments, the additional C-terminal amino acid is glycine. In some embodiments, the RNA polymerase comprises the amino acid sequence SEQ NO: 42. In some embodiments, the RNA polymerase variant comprises an amino acid substitution at position 350, and/or an amino acid substitution at position 351 relative to the wild-type RNA polymerase, wherein the wild-type RNA polymerase comprises the amino acid sequence of SEQ ID NO: 34. In some embodiments, the amino acid substitution at position 350 is E350W. In some embodiments, the amino acid substitution at position 351 is D351V. In some embodiments, the amino acid substitution at position 350 is E350W, and the amino acid substitution at position 351 is D351V. In some embodiments, the RNA polymerase variant comprises the amino acid sequence of SEQ ID NO: 42. In some embodiments, the RNA polymerase variant comprises the amino acid sequence of SEQ ID NO: 43.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows the positive and negative selection strategy used to introduce gene knockouts into E. coli.

FIG. 2 shows the lineage of E. coli strain construction performed in the Examples.

FIG. 3 shows PCR confirmation of Strain 17 genotype. 7 different PCR reactions were performed using Strain 17 colonies as template DNA to confirm PCR amplicons are of expected length as shown in Table 5. 1 kb plus DNA ladder is shown in the lane to the left of lane 1.

FIGS. 4A-4B show T7 RNA polymerase variant 1 expression with commercial BL21 and Strain 11 strains. FIG. 4A shows a total protein gel of Strain 11 and BL21 lysates with overexpression of T7 RNA polymerase variant 1. FIG. 4B shows a growth profile displays final cell density of both Strain 11 and BL21.

FIGS. 5A-5B show the mucoid phenotype in Strain 12 and lack of mucoid phenotype in Strain 24 and Strain 18. FIG. 5A shows the appearance of Strain 12, Strain 24 (BL21 host), and Strain 18 in culture after shake flask fermentation. FIG. 5B shows the appearance of the strains after centrifugation of 100 uL culture in microcentrifuge tubes.

FIG. 6 shows a total protein SDS-PAGE gel image of T7 RNA polymerase variant 1 overexpressed by Strain 17.

FIGS. 7A-7F show online AMBR profiles from fermentation experiments. FIG. 7A shows the CER profile. FIG. 7B shows % dissolved oxygen. FIG. 7C shows the pH profile. FIG. 7D shows base addition. FIG. 7E shows acid pumped. FIG. 7F shows the feeding profile, where induction occurred at hour 9 and feeding began at hour 13.

FIGS. 8A-8B show expression of T7 RNA polymerase variant 2 by Strain 19 (Strain 17 strain transformed with pStrain 23). FIG. 8A shows an SDS-PAGE of T7 RNA polymerase variant 2 expressed and purified from Strain 19 (Strain 17/pStrain 23). FIG. 8B shows specific T7 RNA polymerase variant 2 yield (mg protein per gram biomass) obtained from Strain 19 cell paste and T7 RNA polymerase variant 2 concentration in lysate.

### DETAILED DESCRIPTION

### Genetically Modified Microorganisms

Some aspects of the present disclosure relate to genetically modified microorganisms comprising a genome in which an ompT gene and a lon gene have been mutated, disabled, or deleted. The genome of a microorganism, as used herein, refers to the chromosome or chromosomes of the microorganism, which are long DNA molecules required for cell survival and replication.

The ompT gene encodes the protein OmpT, an aspartyl protease commonly found in the outer membrane of Escherichia coli. When E. coli cells are lysed, OmpT in the outer membrane cleaves proteins that are otherwise stable in the cytoplasm, such as T7 RNA polymerase, reducing the amount of an intact protein that can be isolated from an E. coli cell expressing the protein (see, e.g., Grodberg et al. J Bacteriol. 1988. 170 (3): 1245-1253). Deletion of the ompT gene from the genome thus increases the amount of a protein that can be purified from a cell of a microorganism. An example of a DNA sequence of an ompT gene is given by Accession No. M23630.1 and is reproduced as SEQ ID NO: 27. An example of an amino acid sequence of an OmpT protein is given by Accession No. P09169 and is reproduced as SEQ ID NO: 28.

The lon gene encodes the protein Lon, an ATP-dependent serine protease involved in the degradation of unfolded proteins, including mutant and abnormal proteins. The presence of Lon in cells of a microorganism thus reduces the amount of protein that may be isolated from a cell expressing the protein. An example of a DNA sequence of a lon gene is given by Accession No. M38347.1 and is reproduced as SEQ ID NO: 29. An example of an amino acid sequence of a Lon protein is given by Accession No. POA9MO and is reproduced as SEQ ID NO: 30. A gene or nucleic acid sequence is said to be mutated if it comprises one or more modifications, such as insertions, deletions, or substitutions, relative to a wild-type sequence. An insertion is a modification in which a modified nucleic acid sequence differs from a wild-type nucleic acid sequence by the addition of one or more nucleotides to the wild-type sequence. A deletion is a modification in which a modified nucleic acid sequence differs from the wild-type sequence by the removal of one or more nucleotides. A substitution is a modification in which a modified nucleic acid sequence differs from the wild-type sequence by the change of one nucleotide to a different nucleotide at a position of the sequence. A gene or nucleic acid sequence is said to be disabled if a protein encoded by the gene or nucleic acid sequence has reduced function relative to a protein encoded by a wild-type gene or nucleic acid sequence. A gene or nucleic acid sequence is said to be deleted from a genome if a genome comprising the gene or nucleic acid sequence is modified such that, after being modified, the genome does not comprise the gene or nucleic acid sequence. Mutation or deletion of a promoter operably linked to the gene or nucleic acid sequence often reduces expression of the gene, but if the gene or nucleic acid sequence is present in the genome, then alternative promoters or non-specific transcription by RNA polymerases may result in transcription of the gene or nucleic acid sequence and, consequently, production of the encoded protein. Deletion of the gene or nucleic acid sequence from the genome prevents such mechanisms of transcription, and prevents production of the protein by a cell with a genome in which the gene or nucleic acid sequence has been deleted.

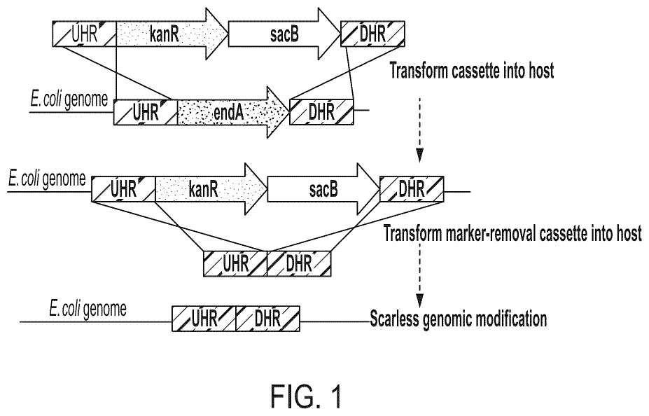

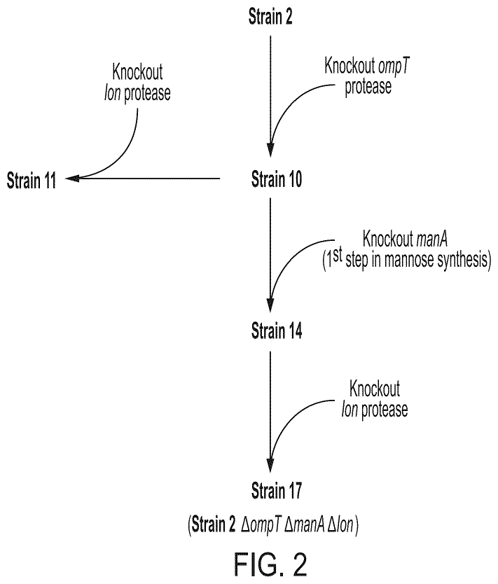

In some embodiments of the genetically modified microorganisms described herein, the microorganism does not express functional ManA. ManA, or mannose-6-phosphate isomerase, is an enzyme encoded by the manA gene that catalyzes the conversion of D-fructose-6-phosphate to D-mannopyranosc 6-phosphate or D-mannose-6-phosphate. This reaction is the first step in the pathway required for the synthesis of GDP-mannose. An example of this synthesis pathway in E. coli is provided in Lee et al., Microb Cell Fact. 2012. 11:48 (see, e.g., FIG. 1). Abrogation of ManA function thus inhibits mannose synthesis, and consequently the synthesis of capsular polysaccharides containing mannose, which contribute to mucoid phenotypes in bacteria. An example of a DNA sequence of a manA gene is given by Accession No. M15380.1 and is reproduced as SEQ ID NO: 31. An example of an amino acid sequence of a ManA protein is given by Accession No. P00946 and is reproduced as SEQ ID NO: 32. Lack of functional ManA in a cell prevents the cell from producing GDP-mannose. Functional ManA refers to a form of ManA that is capable of catalyzing the conversion of D-fructose-6-phosphate to D-mannopyranose 6-phosphate or D-mannose-6-phosphate. A cell is said to lack expression of, or not express, functional ManA if it expresses a form of ManA that does not catalyze the conversion of D-fructose-6-phosphate to D-mannopyranose 6-phosphate or D-mannose-6-phosphate, or if it does not express any ManA proteins.

In some embodiments, the genome comprises a mutation in a manA gene or a promoter operably linked to the manA gene. Mutation, as used herein, refers to a modification of a nucleic acid sequence in which one or more nucleotides is substituted for a different nucleotide (substitution), or one or more nucleotides are added (insertion) or removed (deletion) from a sequence. A mutation in a nucleic acid sequence encoding a protein may change the amino acid sequence of the encoded protein. For example, a mutation that introduces a STOP codon into the coding sequence (nonsense mutation) results in translation of a truncated form of the protein. A mutation that introduces or removes a number of nucleotides other than a multiple of three (e.g. 1, 2, 4, or 5 nucleotides) results in different amino acids being encoded by the nucleic acid sequence starting at the point of the insertion or deletion (frameshift mutation). A mutation that changes a codon encoding a first amino acid to a codon encoding a different amino acid results in the translation of a protein with a different amino acid at the position corresponding to the codon in which the substitution occurred (missense mutation). A mutation in a promoter may prevent RNA polymerase from interacting with the promoter and initiating transcription, and consequently inhibit expression of a nucleic acid sequence to which the promoter is operably linked. A promoter is a nucleic acid sequence that controls expression of a gene or nucleic acid sequence to which it is operably linked. A promoter is said to be operably linked to a gene if the promoter controls the degree to which the gene is expressed. A promoter may be a constitutive promoter, which results in expression of an operably linked gene at a consistent level. A promoter may be a conditional promoter, which regulates expression of an operably linked gene based on environmental conditions, such as the presence, absence, or amount of a stimulus, such as a small molecule, protein, or nucleic acid. In some embodiments, the genome does not comprise a nucleic acid sequence encoding ManA.

In some embodiments of the genetically modified microorganisms described herein, the microorganism does not express functional G6PI. G6PI, or glucose-6-phosphate isomerase, is an enzyme encoded by the pgi gene that catalyzes the reversible isomerization of glucose-6-phosphate to fructose-6-phosphate, which is required for GDP-mannose synthesis. Abrogation of G6PI function thus inhibits mannose synthesis, and consequently the synthesis of capsular polysaccharides containing mannose, which contribute to mucoid phenotypes in bacteria. An example of a DNA sequence of a pgi gene is given by Accession No. X15196 and is reproduced as SEQ ID NO: 49. An example of an amino acid sequence of a G6PI protein is given by Accession No. POA6T1 and is reproduced as SEQ ID NO: 50. Lack of functional G6PI in a cell prevents the cell from isomerizing glucose-6-phosphate to fructose-6-phosphate. Functional G6PI refers to a form of G6PI that is capable of catalyzing isomerization of glucose-6-phosphate to fructose-6-phosphate. A cell is said to lack expression of, or not express, functional G6PI if it expresses a form of G6PI that does not catalyze the isomerization of glucose-6-phosphate to fructose-6-phosphate, or if it does not express any G6PI proteins. In some embodiments, the genome comprises a mutation in a pgi gene or a promoter operably linked to the pgi gene. In some embodiments, the genome does not comprise a nucleic acid sequence encoding G6PI.

In some embodiments of the genetically modified microorganisms described herein, the microorganism does not express functional ManB. ManB, or phosphomannomutase, is an enzyme encoded by the manB gene that catalyzes the conversion of D-mannose 6-phosphate to alpha-D-mannose 1-phosphate, which is required for GDP-mannose synthesis. Abrogation of ManB function thus inhibits mannose synthesis, and consequently the synthesis of capsular polysaccharides containing mannose, which contribute to mucoid phenotypes in bacteria. ManB is also known in the art as CpsG, which is encoded by the cpsG gene. An example of a DNA sequence of a manB gene is given by Accession No. AAC77847 and is reproduced as SEQ ID NO: 51. An example of an amino acid sequence of a ManB protein is given by Accession No. P24175 and is reproduced as SEQ ID NO: 52. Lack of functional ManB in a cell prevents the cell from producing alpha-D-mannose 1-phosphate. Functional ManB refers to a form of ManB that is capable of catalyzing conversion of D-mannose-6-phosphate to alpha-D-mannose 1-phosphate. A cell is said to lack expression of, or not express, functional ManB if it expresses a form of ManB that does not catalyze the conversion of D-mannose-6-phosphate to alpha-D-mannose 1-phosphate, or if it does not express any ManB proteins. In some embodiments, the genome comprises a mutation in a manB gene or a promoter operably linked to the manB gene. In some embodiments, the genome does not comprise a nucleic acid sequence encoding ManB.

In some embodiments of the genetically modified microorganisms described herein, the microorganism does not express functional ManC. ManC, or mannose-1-phosphate guanylyltransferase, is an enzyme encoded by the manC gene that catalyzes the conversion of alpha-D-mannose 1-phosphate to GDP-alpha-D-mannose, which is required for GDP-mannose synthesis. Abrogation of ManC function thus inhibits mannose synthesis, and consequently the synthesis of capsular polysaccharides containing mannose, which contribute to mucoid phenotypes in bacteria. ManC is also known in the art as CpsB, which is encoded by the cpsB gene. An example of a DNA sequence of a manC gene is given by Accession No. AAC77846 and is reproduced as SEQ ID NO: 53. An example of an amino acid sequence of a ManC protein is given by Accession No. P24174 and is reproduced as SEQ ID NO: 54. Lack of functional ManC in a cell prevents the cell from producing GDP-alpha-D-mannose. Functional ManC refers to a form of ManC that is capable of catalyzing conversion of alpha-D-mannose 1-phosphate to GDP-alpha-D-mannose. A cell is said to lack expression of, or not express, functional ManC if it expresses a form of ManC that does not catalyze the conversion of alpha-D-mannose 1-phosphate to GDP-alpha-D-mannose, or if it does not express any ManC proteins. In some embodiments, the genome comprises a mutation in a manC gene or a promoter operably linked to the manC gene. In some embodiments, the genome does not comprise a nucleic acid sequence encoding ManC.

In some embodiments of the genetically modified microorganisms described herein, the microorganism does not express functional Gmd. Gmd, or GDP-mannose 4,6-dehydratase, is an enzyme encoded by the gmd gene that catalyzes the conversion of GDP-alpha-D-mannose to GDP-4-dehydro-6-deoxy-D-mannose, which is required for GDP-L-fucose synthesis. Abrogation of Gmd function thus inhibits fucose synthesis, and consequently the synthesis of capsular polysaccharides containing fucose, which contribute to mucoid phenotypes in bacteria. An example of a DNA sequence of a gmd gene is given by Accession No. AAC77842 and is reproduced as SEQ ID NO: 55. An example of an amino acid sequence of a Gmd protein is given by Accession No. POAC88 and is reproduced as SEQ ID NO: 56. Lack of functional Gmd in a cell prevents the cell from producing GDP-4-dehydro-6-deoxy-D-mannose. Functional Gmd refers to a form of Gmd that is capable of catalyzing conversion of GDP-alpha-D-mannose to GDP-4-dehydro-6-deoxy-D-mannose. A cell is said to lack expression of, or not express, functional Gmd if it expresses a form of Gmd that does not catalyze the conversion of GDP-alpha-D-mannose to GDP-4-dehydro-6-deoxy-D-mannose, or if it does not express any Gmd proteins. In some embodiments, the genome comprises a mutation in a gmd gene or a promoter operably linked to the gmd gene. In some embodiments, the genome does not comprise a nucleic acid sequence encoding Gmd.

In some embodiments of the genetically modified microorganisms described herein, the microorganism does not express functional Fel. Fcl, or GDP-L-fucose synthase, is an enzyme encoded by the fel gene that catalyzes the conversion of GDP-4-dehydro-6-deoxy-D-mannose to GDP-fucose, which is required for GDP-L-fucose synthesis. Abrogation of Fel function thus inhibits fucose synthesis, and consequently the synthesis of capsular polysaccharides containing fucose, which contribute to mucoid phenotypes in bacteria. Fel is also known in the art as WcaG, which is encoded by the wcaG gene. An example of a DNA sequence of an fel gene is given by Accession No. AAC77843 and is reproduced as SEQ ID NO: 57. An example of an amino acid sequence of a Fel protein is given by Accession No. P32055 and is reproduced as SEQ ID NO: 58. Lack of functional Fel in a cell prevents the cell from producing GDP-fucose. Functional Fel refers to a form of Fel that is capable of catalyzing conversion of GDP-4-dehydro-6-deoxy-D-mannose to GDP-fucose. A cell is said to lack expression of, or not express, functional Fel if it expresses a form of Fel that does not catalyze the conversion of GDP-4-dehydro-6-deoxy-D-mannose to GDP-fucose, or if it does not express any Fel proteins. In some embodiments, the genome comprises a mutation in a fel gene or a promoter operably linked to the fel gene. In some embodiments, the genome does not comprise a nucleic acid sequence encoding Fcl.

In some embodiments of the genetically modified microorganisms described herein, the microorganism does not express functional KduI. KduI, or 4-deoxy-L-threo-5-hexosulose-uronate ketol-isomerase, is an enzyme encoded by the kduI gene that catalyzes the isomerization of 5-dehydro-4-deoxy-D-glucuronate to 3-deoxy-D-glycero-2,5-hexodiulosonate, a first step in hexuronate metabolism involved in the synthesis of extracellular polysaccharides that contribute to bacterial mucoid phenotypes. An example of a DNA sequence of a kduI gene is given by Accession No. AAC75882 and is reproduced as SEQ ID NO: 59. An example of an amino acid sequence of a KduI protein is given by Accession No. Q46938 and is reproduced as SEQ ID NO: 60. Lack of functional KduI in a cell prevents the cell from producing 3-deoxy-D-glycero-2,5-hexodiulosonate. Functional KduI refers to a form of KduI that is capable of catalyzing isomerization of 5-dehydro-4-deoxy-D-glucuronate to 3-deoxy-D-glycero-2,5-hexodiulosonate. A cell is said to lack expression of, or not express, functional KduI if it expresses a form of KduI that does not catalyze the isomerization of 5-dehydro-4-deoxy-D-glucuronate to 3-deoxy-D-glycero-2,5-hexodiulosonate, or if it does not express any KduI proteins. In some embodiments, the genome comprises a mutation in a kduI gene or a promoter operably linked to the kduI gene.

In some embodiments, the genome does not comprise a nucleic acid sequence encoding KduI. In some embodiments, the genome comprises a first nucleic acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or up to 100% sequence identity to SEQ ID NO: 23, and a second nucleic acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or up to 100% sequence identity to SEQ ID NO: 25. In some embodiments the genome comprises a third nucleic acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or up to 100% sequence identity to SEQ ID NO: 24.

In some embodiments of the genetically modified microorganisms described herein, the microorganism does not exhibit a mucoid phenotype. A mucoid phenotype, as used herein, refers to a phenotype characterized by excess production of sugars, such as polysaccharides. Microorganism cells with a mucoid phenotype more readily adhere to each other than cells with a non-mucoid phenotype, and liquid in which a microorganism with a mucoid phenotype is growing has a consistency similar to that of mucus (see. e.g., Hamelin et al. J Bacteriol. 1975. 122 (1): 19-24). This mucoid consistency of the liquid medium, and the extracellular polysaccharides present on microorganism cells, interfere with bacterial mixing during fermentation, reduce dissolved oxygen content, and pelleting of the microorganism by centrifugation. Thus, a microorganism without a mucoid phenotype is more easily separated from a liquid medium than a microorganism with a mucoid phenotype.

Escherichia coli

In some embodiments of the genetically modified microorganisms described herein, the microorganism is Escherichia coli (E. coli). In some embodiments, the microorganism is derived from E. coli MG1655. E. coli has been used as a host for plasmid DNA production for many years. Several different strains have been constructed for many different purposes including cloning, plasmid DNA production, and protein expression. Most commonly, E. coli K12 derivatives used such as DH5α, JM108, DH10B and others are used for plasmid DNA cloning and production because they possess specific genomic mutations that are desirable for cloning purposes. These primarily result in the inactivation of genes that encode nucleases, recombinases and other enzymes that reduce DNA stability, purity and cloning efficiency of the strain.

Additionally. E. coli, among other organisms, possess regulatory pathways which limit or modulate expression of other products, which may be desirable to have in larger quantities (e.g., nucleotides). Thus, while the genes controlling these pathways are active, it is difficult to increase the efficiency of the E. coli in producing a desired product.

In some embodiments, the genome does not comprise a nucleic acid sequence encoding endA, the genome does not comprise a nucleic acid sequence encoding recA, and the EcoK1 restriction system has been inactivated. The endA gene encodes endonuclease-1 protein, which when expressed induces double-strand break activity. This activity will degrade and otherwise compromise the production of plasmid DNA by E. coli possessing the gene. The recA gene encodes the RecA protein, which is a key protein for the repair and maintenance of DNA. However, RecA through its properties in facilitating DNA repair, plays a central role in the homologous recombination of DNA, as well as mediate homology pairing, homologous recombination. DNA break repair, and the SOS response, wherein DNA damage triggers the cell cycle to arrest initiate DNA repair and mutagenesis. The properties of both EndA and RecA are not beneficial in the production of consistent and identical DNA plasmids.

Native E. coli possess the EcoKI restriction system. EcoKI is a restriction-modification enzyme complex responsible for identifying and restricting unmethylated, foreign DNA, and for modifying native, hemimethylated DNA by methylation for self-identification. Left alone, the EcoKI system will recognize non-methylated DNA as foreign and, if the DNA also possesses unique EcoKI-recognition sites, degrade it. While it is not essential to inactivate the EcoKI system from E. coli to clone plasmid DNA, deletion does significantly increase cloning and transformation efficiencies if the desired plasmid DNA possesses EcoKI recognition sites.

In some embodiments, the genotype of the microorganism is ΔendA ΔrecA Δ(mrr-hsdRMS-symE-mcrBC) ΔompT ΔmanA Δlon. mrr encodes Mrr, a restriction endonuclease that cleaves methylated DNA at specific recognition sites. The hsdRMS operon encodes HsdR, HsdM, and HsdS, three protein components of the EcoK1 restriction system, which cleaves unmethylated DNA at specific recognition sites. symE encodes SymE, an endoribonuclease, merBC encodes MerBC, a restriction endonuclease that cleaves methylated DNA at specific recognition sites.

### Vectors Encoding Recombinant Proteins

The genetically modified microorganisms described herein, in some embodiments, comprise a nucleic acid sequence encoding a recombinant protein. A “recombinant protein” as used herein, refers to a protein encoded by a nucleic acid sequence that has been cloned into an expression vector, such as a plasmid, and expressed from that vector by the transcription of mRNA from the vector and translation of the resulting mRNA.

In some embodiments, the nucleic acid sequence encoding the recombinant protein is located in the genome of the microorganism. A nucleic acid is said to be located in the genome if the genome of the organism comprises the nucleic acid sequence. In some embodiments, the nucleic acid sequence encoding the recombinant protein is located on a plasmid. A plasmid refers to a circular DNA molecule that is separate from the chromosome of a microorganism.

In some embodiments, the recombinant protein encoded by the nucleic acid sequence is an RNA polymerase. An RNA polymerase is a protein that binds to a template polynucleotide and synthesizes an RNA polynucleotide, or transcript, that comprises an RNA sequence that is complementary to a sequence in the template polynucleotide. In some embodiments, the RNA polymerase is a DNA-dependent RNA polymerase, which synthesizes an RNA transcript from a DNA template polynucleotide. In some embodiments, the RNA polymerase is an RNA-dependent RNA polymerase, which synthesizes an RNA transcript from an RNA template polynucleotide. RNA polymerases include but are not limited to, a phage RNA polymerase, e.g., a T7 RNA polymerase, a T3 RNA polymerase, an SP6 RNA polymerase, a K11 RNA polymerase, and/or mutant polymerases such as, but not limited to, polymerases able to incorporate modified nucleic acids. As a non-limiting example, the RNA polymerase may be modified to exhibit an increased ability to incorporate a 2′-modified nucleotide triphosphate compared to an unmodified RNA polymerase.

In some embodiments, the RNA polymerase is a T7 RNA polymerase, T3 RNA polymerase, SP6 RNA polymerase, or K11 RNA polymerase. An example of a DNA sequence encoding a T7 RNA polymerase is given by Accession No. M38308.1 and is reproduced as SEQ ID NO: 33. An example of an amino acid sequence of a T7 RNA polymerase is given by Accession No. P00573 and is reproduced as SEQ ID NO: 34. An example of a DNA sequence encoding a T3 RNA polymerase is given by Accession No. X02981.1 and is reproduced as SEQ ID NO: 35. An example of an amino acid sequence of a T3 RNA polymerase is given by Accession No. P07659 and is reproduced as SEQ ID NO: 36. An example of a DNA sequence encoding an SP6 RNA polymerase is given by Accession No. Y00105.1 and is reproduced as SEQ ID NO: 37. An example of an amino acid sequence of an SP6 RNA polymerase is given by Accession No. P06221 and is reproduced as SEQ ID NO: 38. An example of a DNA sequence encoding a K11 RNA polymerase is given by Accession No. X53238.1 and is reproduced as SEQ ID NO: 39. An example of an amino acid sequence of a K11 RNA polymerase is given by Accession No. P18147 and is reproduced as SEQ ID NO: 40.

In some embodiments, the RNA polymerase is an RNA polymerase variant. RNA polymerase variants include at least one amino acid substitution, relative to the wild type (WT) RNA polymerase. For example, with reference to WT T7 RNA polymerase having an amino acid sequence of SEQ ID NO: 34, the glycine at position 47 is considered a “wild-type amino acid.” whereas a substitution of the glycine for alanine at position 47 is considered an “amino acid substitution” that has a high-helix propensity. In some embodiments, the RNA polymerase variant is a T7 RNA polymerase variant comprising at least one (one or more) amino acid substitution relative to WT RNA polymerase (e.g., WT T7 RNA polymerase having an amino acid sequence of SEQ ID NO: 34).

In some embodiments, a RNA polymerase variant comprises a RNA polymerase that includes an (at least one) amino acid modification causes a loop structure of the RNA polymerase variant to undergo a conformational change to a helix structure as the RNA polymerase variant transitions from an initiation complex to an elongation complex. In some embodiments, the RNA polymerase variant comprises (a) an amino acid substitution at a binding site residue for de novo RNA synthesis; and (b) an amino acid modification that causes increased transcription efficiency, relative to wild-type RNA polymerase. In some embodiments, the amino acid modification is an amino acid substitution at position 47, relative to the wild-type RNA polymerase, wherein the wild-type RNA polymerase comprises the amino acid sequence of SEQ ID NO: 34. The amino acid substitution, in some embodiments, is a high propensity amino acid substitution. Examples of high-helix propensity amino acids include alanine, isoleucine, leucine, arginine, methionine, lysine, glutamine, and/or glutamate. In some embodiments, the amino acid substitution at position 47 is G47A. In some embodiments, an RNA polymerase variant comprises an RNA polymerase that includes an additional C-terminal amino acid, relative to the wild-type RNA polymerase. The additional C-terminal amino acid, in some embodiments, is selected from glycine, alanine, threonine, proline, glutamine, serine. In some embodiments, the additional C-terminal amino acid (e.g., at position 884 relative to wild-type RNA polymerase comprising the amino acid sequence of SEQ ID NO: 34) is glycine. In some embodiments, the RNA polymerase variant comprises the amino acid sequence of SEQ ID NO: 41. In some embodiments, the RNA polymerase variant comprises an amino acid substitution at position 350, and/or an amino acid substitution at position 351 relative to the wild-type RNA polymerase, wherein the wild-type RNA polymerase comprises the amino acid sequence of SEQ ID NO: 34. In some embodiments, the amino acid substitution at position 350 is E350W. In some embodiments, the amino acid substitution at position 351 is D351V. In some embodiments, the RNA polymerase variant comprises the amino acid sequence of SEQ ID NO: 42. In some embodiments, the RNA polymerase variant comprises the amino acid sequence of SEQ ID NO: 43.

### Methods of Recombinant Protein Production

Some aspects of the disclosure relate to a method for producing a polypeptide or protein, comprising the steps of i) introducing a nucleic acid molecule comprising a sequence encoding a polypeptide or protein into one of the genetically modified microorganisms described herein; ii) culturing the genetically modified organism under conditions suitable for expression of the polypeptide or protein; and iii) isolating the polypeptide or protein.

Introducing a vector into a microorganism refers to contacting a microorganism with the vector or donor organism under conditions that result in the incorporation of the vector into the cell of the microorganism. A vector may be introduced by transformation, a process in which a cell acquires extracellular DNA from the environment. Transformation may be accomplished through electroporation, or electropermeabilization, a process in which a composition comprising extracellular DNA and a microorganism cell is subjected to a pulse of electricity, which transiently opens pores in the cell wall and cell membrane(s), allowing DNA to enter the cytoplasm of the cell. Transformation may also be accomplished by subjecting a composition comprising extracellular DNA and competent microorganism cells to heat shock, in which the composition is rapidly heated and cooled, which results in entry of extracellular DNA into the cytoplasm of the competent cells. A cell is said to be competent if it is capable of acquiring extracellular DNA from the environment. Cells may be naturally competent, or capable of acquiring extracellular DNA, or induced to be competent, such as by incubation with one or more compounds that facilitate the process of DNA acquisition. For example, chemically competent cells are cells treated with a salt, dimethyl sulfoxide (DMSO), and/or polyethylene glycol (PEG). Salts, such as RbCl, MgCl₂, and/or CaCl₂), neutralize the negative charge of phospholipids of the cell membrane the phosphate backbone of DNA, allowing DNA to associate with the surface of a cell instead of being repelled. DMSO weakens the lipid bilayer of the cell membrane, reducing its thickness and increasing its permeability. Cells that are made chemically competent by treatment with salt. DMSO, and/or PEG are thus more likely than unmodified cells to acquire extracellular DNA.

A vector may be introduced by conjugation, a process in which a donor organism introduces the vector into the cell of a recipient organism, such as the microorganism. In conjugation, a donor organism cell comprising a pilus, or hair-like appendage, contacts a recipient organism cell, and the pilus forms a tunnel that connects the interior of both cells. One DNA strand of the vector is then transferred through the pilus into the recipient cell. Following this transfer, both cells, which each contain a single-stranded form of the vector, synthesize the complementary strand, leaving each cell with a double-stranded form of the vector.

A vector may be introduced by transduction, a process in which DNA is introduced to a cell by a virus or viral vector. Infection of a microorganism by a virus, such as a bacteriophage, introduces viral DNA into the cytoplasm of the microorganism cell. If the bacteriophage contains the DNA sequence of a vector or plasmid, then the microorganism can will contain the vector plasmid after infection, maintaining and replicating the plasmid during cell division (see, e.g. Ammann et al. J Bacteriol. 2008. 190 (8): 3083-3087).

Culturing a microorganism refers to incubating the microorganism in an environment that permits growth and replication of the microorganism. The environment that permits a microorganism's growth and replication depends on the microorganism. The environment may be a liquid or solid medium containing nutrients that the microorganism uses for growth and replication. Non-limiting examples of media that may be used to culture a microorganism include lysogeny broth, Luria-Bertani (LB) broth or agar medium, tryptone soy (TS) broth or agar medium, and Todd-Hewitt broth or agar medium.

Conditions suitable for expression of the polypeptide or protein depend on the microorganism, the vector and/or nucleic acid sequence encoding the polypeptide or protein, and the polypeptide or protein itself. As described in the preceding paragraph, the environment in which the microorganism is cultured must support its growth and replication, and may be one of the liquid or solid media known in the art.

If the nucleic acid sequence encoding the polypeptide or protein is present on a vector, such as a plasmid, then the vector must be maintained in the cells of the microorganism for the polypeptide or protein to be expressed. Methods of maintaining a vector in a microorganism are well known in the art. One method of maintaining a vector in a microorganism, if the vector encodes a protein that makes a cell resistant to the action of antibiotic, involves culturing the microorganism in the presence of an antibiotic that kills or inhibits the growth of cells that do not contain the vector, but permits growth and replication of cells that do contain the vector.

If the nucleic acid sequence is operably linked to a conditional promoter, then expression of the polypeptide or protein requires that the microorganism be cultured in conditions in which the conditional promoter is active. For example, the lac operon in many bacteria is active only when lactose is present in the cell. A lac repressor protein complex binds to the lac operator, a nucleic acid sequence located between the lac promoter and the genes encoded by the lac operon, and prevents transcription of genes encoded in the lac operon. Allolactose, an isomer of lactose, binds to lac repressor, inducing a conformational change that results in release of lac repressor from the operator, which allows transcription of the encoded genes. If a lac operator is present upstream of the nucleic acid sequence encoding the gene or polypeptide to be expressed, then the polypeptide or protein will be expressed only in the presence of allolactose or a similar compound, such as isopropyl-β-D-thiogalactopyranoside (IPTG), and expression can be controlled by modulating the amount of IPTG in the culture environment (see, e.g., Donovan et al. J Ind Microbiol. 1996. 16 (3): 145-54).

Methods of isolating a polypeptide or protein are well known in the art (see, e.g., Wingfield. Curr Protoc Protein Sci. 2016. 80:6.1.1-6.1.35). Generally, bacteria expressing the protein are pelleted by centrifugation to separate bacterial cells from liquid medium. Then, bacterial cells are lysed, such as by mechanical French press or enzymatically by lysozyme treatment. Bacterial lysate is centrifuged to pellet cellular components. If the recombinant protein is present in the cytoplasm or periplasm of bacterial cells, then the protein is purified from the lysate supernatant. If the recombinant protein is present in inclusion bodies, then the lysate pellet is treated with Triton, EDTA, and/or urea to extract cell wall components, and the remaining inclusion bodies are solubilized. Denatured protein may then be purified by from the solubilized inclusion bodies, and, if necessary, refolded into a desired conformation.

Some aspects of the disclosure relate to a method for producing a polypeptide or protein, comprising the steps of i) culturing one of the genetically modified microorganisms described herein under conditions suitable for expression of the polypeptide or protein; and ii) isolating the polypeptide or protein.

In some embodiments of the methods described herein, the polypeptide or protein is an RNA polymerase. In some embodiments, the RNA polymerase is T7 RNA polymerase, T3 RNA polymerase, SP6 RNA polymerase, or K11 RNA polymerase. In some embodiments, the RNA polymerase is an RNA polymerase variant. RNA polymerase variants include at least one amino acid substitution, relative to the wild type (WT) RNA polymerase. For example, with reference to WT T7 RNA polymerase having an amino acid sequence of SEQ ID NO: 34, the glycine at position 47 is considered a “wild-type amino acid,” whereas a substitution of the glycine for alanine at position 47 is considered an “amino acid substitution” that has a high-helix propensity. In some embodiments, the RNA polymerase variant is a T7 RNA polymerase variant comprising at least one (one or more) amino acid substitution relative to WT RNA polymerase (e.g., WT T7 RNA polymerase having an amino acid sequence of SEQ ID NO: 34).

In some embodiments, a RNA polymerase variant comprises a RNA polymerase that includes an (at least one) amino acid modification causes a loop structure of the RNA polymerase variant to undergo a conformational change to a helix structure as the RNA polymerase variant transitions from an initiation complex to an elongation complex. In some embodiments, the RNA polymerase variant comprises (a) an amino acid substitution at a binding site residue for de novo RNA synthesis; and (b) an amino acid modification that causes increased transcription efficiency, relative to wild-type RNA polymerase. In some embodiments, the amino acid modification is an amino acid substitution at position 47, relative to the wild-type RNA polymerase, wherein the wild-type RNA polymerase comprises the amino acid sequence of SEQ ID NO: 34. The amino acid substitution, in some embodiments, is a high propensity amino acid substitution. Examples of high-helix propensity amino acids include alanine, isoleucine, leucine, arginine, methionine, lysine, glutamine, and/or glutamate. In some embodiments, the amino acid substitution at position 47 is G47A.

In some embodiments, an RNA polymerase variant comprises an RNA polymerase that includes an additional C-terminal amino acid, relative to the wild-type RNA polymerase. The additional C-terminal amino acid, in some embodiments, is selected from glycine, alanine, threonine, proline, glutamine, serine. In some embodiments, the additional C-terminal amino acid (e.g., at position 884 relative to wild-type RNA polymerase comprising the amino acid sequence of SEQ ID NO: 34) is glycine. In some embodiments, the RNA polymerase variant comprises the amino acid sequence of SEQ ID NO: 41. In some embodiments, the RNA polymerase variant comprises an amino acid substitution at position 350, and/or an amino acid substitution at position 351 relative to the wild-type RNA polymerase, wherein the wild-type RNA polymerase comprises the amino acid sequence of SEQ ID NO: 34. In some embodiments, the amino acid substitution at position 350 is E350W. In some embodiments, the amino acid substitution at position 351 is D351V. In some embodiments, the RNA polymerase variant comprises the amino acid sequence of SEQ ID NO: 42. In some embodiments, the RNA polymerase variant comprises the amino acid sequence of SEQ ID NO: 43.

### Nucleic Acids

A “nucleic acid” is at least two nucleotides covalently linked together, and in some instances, may contain phosphodiester bonds (e.g., a phosphodiester “backbone”). As used herein, the terms “nucleic acid sequence” and “polynucleotide” are used interchangeably and do not imply any length restriction. As used herein, the terms “nucleic acid” and “nucleotide” are used interchangeably. The terms “nucleic acid sequence” and “polynucleotide” embrace DNA (including cDNA) and RNA sequences. The nucleic acid sequences described herein include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

Engineered nucleic acids may be produced using standard molecular biology methods. In some embodiments, engineered nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

The nucleic acid vectors described herein also may have one or more terminator sequences present or removed. A terminator sequence is a nucleic acid sequence that signals the end of the expression cassette or transcribed region. For effective transcription vectors typically include one or more terminator sequences. Terminator sequences include, for instance. T7 and T4 terminator sequences.

The preferred vectors described herein may also have a resistant marker, or a marker that is unique to the particular vector. For instance, the vector may have originally had an ampicillin resistant marker. In some embodiments described herein, the ampicillin marker is replaced with a different marker such as kanamycin resistant marker. In some embodiments, the E. coli genomes disclosed herein may further express a gene for a positive selection marker based on a first environmental factor or a negative selection maker based on a second environmental factor, wherein the first and second environmental factors are not the same. In some embodiments, the E. coli genomes disclosed herein may further express a gene for a positive selection marker based on a first environmental factor and a negative selection maker based on a second environmental factor, wherein the first and second environmental factors are not the same. In some embodiments, the positive selection marker is a gene capable of conferring kanamycin resistance. In some embodiments, the negative selection marker is a gene capable of expressing levansucrase.

A vector disclosed herein may also have any pathogen-derived sequences removed. Removal of pathogen derived sequences can have a positive effect on the product yield.

The origin of replication (ori) is included in the nucleic acid described herein and may be modified as disclosed herein. The nucleic acid may in some embodiments contain several ori, for example 2 ori's. It can, for example, be a combination of a low-copy ori and a temperature-dependent ori or for example ori's that allow propagation in various host organisms.

The nucleic acids may also contain one or more elements from other known vectors. For example other vectors include phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In other embodiments the nucleic acids described herein do not include any elements from any one or more of the other vectors.

When applied to a nucleic acid sequence, the term “isolated” in the context used herein denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

There are many established algorithms available to align two nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of nucleic acid sequences for comparison may be conducted, for example, by computer implemented algorithms (e.g., GAP, BESTFIT, FASTA or TFASTA), or BLAST and BLAST 2.0 algorithms.

In a sequence identity comparison, the identity may exist over a region of the sequences that is at least 10 nucleic acid residues in length (e.g. at least 15, 20, 30, 40, 50, 75, 100, 150, 200, 250, 300, 350, 400, 450, 500, 550, 600, 650 or 685 nucleotides in length, e.g. up to the entire length of the reference sequence.

Substantially homologous or substantially identical nucleic acids have one or more nucleotide substitutions, deletions, or additions. In many embodiments, those changes are of a minor nature, for example, involving only conservative nucleic acid substitutions that may result in the same amino acid being coded for during translation or in a different but conservative amino acid substitution. Conservative amino acid substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine. Substantially homologous nucleic acids also encompass those comprising other substitutions that do not significantly affect the folding or activity of a translation product.

The nucleic acid vectors described herein may be empty vectors or may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide. An expression cassette encodes an RNA including at least the following elements: a 5′ untranslated region, an open reading frame region encoding the mRNA, a 3′ untranslated region and a polyA tail. The open reading frame may encode any mRNA.

A “5′ untranslated region (UTR)” refers to a region of an mRNA that is directly upstream (i.e., 5′) from the start codon (i.e., the first codon of an mRNA transcript translated by a ribosome) that does not encode a protein or peptide.

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

A “polyA tail” is a region of mRNA that is downstream. e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly (A) tail functions to protect mRNA from enzymatic degradation, e.g., in the cytoplasm, and aids in transcription termination, export of the mRNA from the nucleus, and translation.

One of ordinary skill in the art appreciates that different species exhibit “preferential codon usage”. As used herein, the term “preferential codon usage” refers to codons that are most frequently used in cells of a certain species, thus favoring one or a few representatives of the possible codons encoding each amino acid. For example, the amino acid threonine (Thr) may be encoded by ACA. ACC. ACG, or ACT, but in mammalian host cells ACC is the most commonly used codon; in other species, different Thr codons may be preferential. Preferential codons for a particular host cell species can be introduced into the polynucleotides described herein by a variety of methods known in the art. Alternatively non-preferred codons may be used. In some embodiments, the nucleic acid sequence is codon optimized. Methods for codon optimization are known in the art.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of the polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide).

A “nucleic acid vector” is a polynucleotide that carries at least one foreign or heterologous nucleic acid fragment. A nucleic acid vector may function like a “molecular carrier”, delivering fragments of nucleic acids respectively polynucleotides into a host cell or as a template for IVT. An “in vitro transcription template (IVT),” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a poly A tail. The particular nucleotide sequence composition and length of an IVT template will depend on the mRNA of interest encoded by the template.

In some embodiments, the nucleic acid vector described herein is a circular nucleic acid such as a plasmid. In other embodiments it is a linearized nucleic acid. According to one embodiment the nucleic acid vector comprises a predefined restriction site, which can be used for linearization of the vector. Intelligent placement of the linearization restriction site is important, because the restriction site determines where the vector nucleic acid is opened/linearized. The restriction enzymes chosen for linearization should preferably not cut within the critical components of the vector.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

### EXAMPLES

### Example 1: Production of T7 RNA Polymerase Variant 1 and T7 RNA Polymerase Variant 2 in Genetically Modified E. coli Strain Deficient in Lon, OmpT, and ManA

### Introduction

The goal of the work described in this Example was to generate an E. coli strain for expression of a recombinant protein. Both lon and ompT ORFs were completely removed from Strain 2, a strain that accepts and maintains plasmid DNA, and can be purified from the host due to the removal of the EcoKI restriction system, endA and recA from the genome. A robust strain for plasmid-borne enzyme expression that has significantly reduced protease activity, and therefore produces recombinant enzymes at high purity was successfully produced.

### Methods

TABLE 1
Strains used in this Example.

| Strain Name | Strain Genotype | Plasmid |
|---|---|---|
| Strain 1 | ΔendA ΔrecA | None |
| Strain 9 | ΔendA ΔrecA | pStrain 21 |
| Strain 5 | ΔendA ΔrecA Δ(mrr-hsdRMS- | pStrain 21 |
| | symE-mcrBC)::kan-sacB | |
| Strain 2 | ΔendA ΔrecA Δ(mrr-hsdRMS- | None |
| | symE-mcrBC) | |
| Strain 10 | ΔendA ΔrecA Δ(mrr-hsdRMS- | None |
| | symE-mcrBC) ΔompT | |
| Strain 11 | ΔendA ΔrecA Δ(mrr-hsdRMS- | None |
| | symE-mcrBC) ΔompT Δlon | |
| Strain 12 | ΔendA ΔrecA Δ(mrr-hsdRMS- | pStrain 2 |
| | symE-mcrBC) ΔompT Δlon | |
| Strain 13 | ΔendA ΔrecA Δ(mrr-hsdRMS- | pStrain 21 |
| | symE-mcrBC) ΔompT | |
| | ΔmanA::kan-sacB | |
| Strain 14 | ΔendA ΔrecA Δ(mrr-hsdRMS- | pStrain 21 |
| | symE-mcrBC) ΔompT ΔmanA | |
| Strain 15 | ΔendA ΔrecA Δ(mrr-hsdRMS- | None |
| | symE-mcrBC) ΔompT ΔmanA | |
| | Δlon::kan-sacB | |
| Strain 16 | ΔendA ΔrecA Δ(mrr-hsdRMS- | pStrain 21 |
| | symE-mcrBC) ΔompT ΔmanA | |
| | Δlon::kan-sacB | |
| Strain 17 | ΔendA ΔrecA Δ(mrr-hsdRMS- | None |
| | symE-mcrBC) ΔompT ΔmanA | |
| | Δlon | |
| Strain 18 | ΔendA ΔrecA Δ(mrr-hsdRMS- | pStrain 2 |
| | symE-mcrBC) ΔompT ΔmanA | |
| | Δlon | |
| Strain 19 | ΔendA ΔrecA Δ(mrr-hsdRMS- | pStrain 23 |
| | symE-mcrBC) ΔompT ΔmanA | |
| | Δlon | |
| Strain 20 | ΔendA ΔrecA Δ(mrr-hsdRMS- | pStrain 23 |
| | symE-mcrBC) ΔompT ΔmanA | |
| | Δlon | |

TABLE 2
Plasmids used in this Example.

| Plasmid | |
|---|---|
| Name | Plasmid Genotype |
| pStrain 21 | \|<repA101\|ori101_ts\|<recA\|<bla\|<tetR\|<P(tetR)\|P(tet)> |
| | \|gamma>\|beta>\|exo>60a> |
| pStrain 22 | [R6K>\|Ori_R6Kgamma\|ompT UHR \| ompT DHR \| kanR> |
| | \| SacB> \| |
| pStrain 2 | \|pUC>\|term_rpoC\|term_bla\|P(T5)>\| T7 RNA polymerase |
| | variant 1>P(amp)>\|kan>\|P(lacI)>\|lacI\| |
| pStrain 23 | \|pUC>\|term_rpoC\|term_bla\|P(T5)>\| T7 RNA polymerase |
| | variant 2>\|P(amp)>\|kan>\|P(lacI)>\|lacI\| |
| pStrain 24 | \|pUC>\|term_rpoC\|term_bla\|P(T5)>\| T7 RNA polymerase |
| | variant 3>P(amp)>\|kan>\|P(lacI)>\|lacI\| |

TABLE 3
Primers used in this Example.

| Primer | Sequence (5′-3′) | Note |
|---|---|---|
| KS786 | /5Phos/AAAAATACATATTCAATCATTAAAACGATTGAAT | KO cassette for ompT |
| | GGAGAACTTTTGTGTAGGCTGGAGCTGCTTC (SEQ ID | |
| | NO: 1) | |
| KS787 | T*T*C*C*C*C*GGGGCGATTTTCACCTCGGGGAAATTTTA | KO cassette for ompT |
| | GTTGGCGTTCTTAATTCTCATGTTTGACAG (SEQ ID | |
| | NO: 2) | |
| KS651 | AACGGATAAGACGGGCATAAATGAGG (SEQ ID NO: 3) | Popout cassette for |
| | | ΔompT::kan-sacB |
| KS652 | GTTTAATAAAAAAAAGATTAAGGGATGAAGGAAC (SEQ | Popout cassette for |
| | ID NO: 4) | ΔompT::kan-sacB |
| KS1727 | /5Phos/GCTTTAATAGTGGGATTAATTTCCACATTAAAAC | KO cassette for manA |
| | AGGGATTGATCGTGTAGGCTGGAGCTGCTTCGG (SEQ ID | |
| | NO: 5) | |
| KS1728 | C*C*T*T*TAATAAGCTTAGCAAGAGATGTTAATTTTTTCA | KO cassette for manA |
| | GTAAGCTCTTAATTCTCATGTTTGACAGGGTACCCAT | |
| | (SEQ ID NO: 6) | |
| KS1729 | /5Phos/GATGTCGGAGTTACGCAGGAATTG (SEQ ID | Popout cassette for |
| | NO: 7) | ΔmanA::kan-sacB |
| KS1730 | T*A*T*C*AAACAGTGGTTTGCTTACTTCGTTATTAAC | Popout cassette for |
| | (SEQ ID NO: 8) | ΔmanA::kan-sacB |
| KS1132 | /5Phos/CCCATACAATTAGTTAACCAAAAAGGGGGGATTT | KO cassette for lon |
| | TATCTCCCCTTGTGTAGGCTGGAGCTGCTTCGG (SEQ ID | |
| | NO: 9) | |
| KS1133 | GCCTGCCAGCCCTGTTTTTATTAGTGCATTTTGCGCGAGGT | KO cassette for lon |
| | CACGTTAATTCTCATGTTTGACAGGGTACCCAT (SEQ ID | |
| | NO: 10) | |
| KS1260 | /5Phos/TCTGATTCAGATCCTCAAAGAGCCG (SEQ ID | Popout cassette for |
| | NO: 11) | Δlon::kan-sacB |
| KS1261 | G*G*C*A*G*C*ACGCTCTTTAACGGC (SEQ ID NO: | Popout cassette for |
| | 12) | Δlon::kan-sacB |
| KS518 | CATCAGTTCGAACTGCAATCCGTG (SEQ ID NO: 13) | PCR confirm endA |
| KS519 | CATCTTCGCAAAGCGCGATG (SEQ ID NO: 14) | PCR confirm endA |
| KS520 | GGCGGCAGTGAAGAGAAGCCT (SEQ ID NO: 15) | PCR confirm recA |
| KS521 | GCAATAACGCGCTCGTAATCTTCTG (SEQ ID NO: 16) | PCR confirm recA |
| KS1431 | CGGCGTACCGCAACACTTTTG (SEQ ID NO: 17) | PCR confirm purR |
| KS1432 | TGTCGCCTGCGTTAACCTGGTT (SEQ ID NO: 18) | PCR confirm purR |
| KS1112 | GCGTTGTTGGGATCAATTCTGAGAC (SEQ ID NO: 19) | PCR confirm EcoKI |
| KS1113 | TGCTGACCAAAACCGACGTCG (SEQ ID NO: 20) | PCR confirm EcoKI |
| KS651 | AACGGATAAGACGGGCATAAATGAGG (SEQ ID NO: 3) | PCR confirm ompT |
| KS652 | GTTTAATAAAAAAAAGATTAAGGGATGAAGGAAC (SEQ | PCR confirm ompT |
| | ID NO: 4) | |
| KS1729 | GATGTCGGAGTTACGCAGGAATTG (SEQ ID NO: 7) | PCR confirm manA |
| KS1730 | TATCAAACAGTGGTTTGCTTACTTCGTTATTAAC (SEQ | PCR confirm manA |
| | ID NO: 8) | |
| KS979 | GTCGCAACGTTGAATGAACTGAGC (SEQ ID NO: 21) | PCR confirm lon |
| KS980 | AGCTCGGTACTTTAGCAGCAGCGA (SEQ ID NO: 22) | PCR confirm lon |

‘*’ denotes phosphorothioate modification of preceding nucleotide
‘/5Phos/’ denotes 5′ phosphorylation of 5′ terminal nucleotide

TABLE 4
gBlocks used in this Example

| KS gBlock # | DNA sequence (5′-3′) | Note |
|---|---|---|
| KS gBlock | AACGGATAAGACGGGCATAAATGAGGAAGAAATGGCGCGCCCTGC | Template to |
| 165 | AGGATTCGAACCTGCGGCCCACGACTTAGAAGTTCCTAGAACGAC | amplify ompT |
| | ATTTTAAGTCAACAACTTACCGCGCCATCTCTGCGCTCACACGTC | popout |
| | CCACTACCTCAAAACATGTAAAGCCTTGCAAGCCATTGCGAGGCC | cassette |
| | TTATGTGTCTCAGTTTTGTCCCTCTTTTTTGTACTAAAAAACATA | |
| | GTAATTGAGGATAAACCTCATGCTATTTTCGCTTATATGCCTCTA | |
| | AAGGCATGGCACTTAAATAGATAAAAGCACCACAAAAGCATAAAA | |
| | AAACCACACAGTAAAACCGAAATATGAAACAATAACAGATAATTA | |
| | AACCAAAAACAGATAGCGCATTGTGATAATCATTCAATACTAAAC | |
| | AAAATATAAACAGTGGAGCAATATGTAATTGACTCATTAAGTTAG | |
| | ATATAAAAAATACATATTCAATCATTAAAACGATTGAATGGAGAA | |
| | CTTTTGAACGCCAACTAAAATTTCCCCGAGGTGAAAATCGCCCCG | |
| | GGGAATAACTAGCCATTTCAATGTAACAATTAACCCTTAAAATAA | |
| | ACCCAGAAGGTTATTAACTAAATCACATAGAAAACCATCAATTAT | |
| | AGTATGTATAAAATAGGCGACAGCAACCCAATTACAAATTAATGG | |
| | TTCCAGAATATCACATCAAAAAAAACGCTGTATAATATTATAATT | |
| | AACATGTAGACAACTTGTAATAAACATTATCAGTCAATTGTTTTG | |
| | TTTATTCCATCTGTGACGCCGATTATTTTCTCAAAATAATGAGAT | |
| | GGCGTGACACCATAATAATCTTTAAATGCACATATGAAATATGAA | |
| | GTACTGTTATAGCCACATTTCTGGGCTACGACATTGATAGAATAA | |
| | GAGTTTGAAGTTATGAGTTTTTTTGCATACCTCATCCTAGTATCT | |
| | CTCAATATTTCAGTAAATGACGTTCCTTCATCCCTTAATCTTTTT | |
| | TTTATTAAAC (SEQ ID NO: 23) | |
| KS gBlock | ACGCCTTTCGCCGCGATGTCGGAGTTACGCAGGAATTGCAGCGCC | Template to |
| 659 | ACATATTTATCATTTTCGCTGGCCTCCGGGTCACGCAGAATGTCG | amplify |
| | GCCACCTGTTGTTGGTGCGCCGGACGCAGCATGAACGACGCATCA | manA popout |
| | TGAAACGCCTGGCGCGCCAACAGAGTTAACGCTTCGGGTGCGACT | cassette |
| | TTCAAAATCTCCTGCCCTTCAAATTCAGATACGCTAACGTGTTCG | |
| | CTGGTTAGCAGGTAATACTCAGTATCATCTTTTTTGAGTGGAAAA | |
| | GGAGCCTGATAATGAAAGGGTTTGTTTGACATTGTTCTCTCACTT | |
| | ACTGCCTGGTTTGGTTATGCTCTGGGCGGGTGTTCCGTTGCCCTG | |
| | TTAAAAGCGAGTAACAATATCCTACACACTTTTTTAACAAAAACT | |
| | GAGACTAGTACGACTTTTTGCGGCTCCAGGTTACTTCCCGTAGGA | |
| | TTCTTGCTTTAATAGTGGGATTAATTTCCACATTAAAACAGGGAT | |
| | TGATCGAGCTTACTGAAAAAATTAACATCTCTTGCTAAGCTTATT | |
| | AAAGGCTTATAACACCTTCAGGCGGCCAGTCCGCCTGATTTCATT | |
| | TTATGGATAATCATTATGAATAAATCGCTGGTAGCGGTAGGCGTC | |
| | ATTGTTGCGCTAGGCGTAGTCTGGACAGGCGGCGCATGGTATACA | |
| | GGCAAGAAGATTGAAACCCATCTCGAAGACATGGTCGCGCAGGCG | |
| | AACGCGCAACTCAAACTGACAGCTCCTGAATCCAACCTGGAAGTG | |
| | AGTTATCAAAACTATCATCGCGGCGTATTCAGCAGCCAGTTGCAA | |
| | CTGTTGGTGAAACCCATTGCCGGGAAAGAAAATCCGTGGATTAAA | |
| | AGCGGTCAGAGCGTCATCTTCAACGAATCGGTTGATCATGGTCCC | |
| | TTCCCGCTTGCCCAGCTTAAAAAACTGAACCTGATCCCGTCGATG | |
| | GCATCAATTCAAACCACGCTGGTTAATAACGAAGTAAGCAAACCA | |
| | CTGTTTGATA (SEQ ID NO: 24) | |
| KS gBlock | TCTGATTCAGATCCTCAAAGAGCCGAAAAACGCCCTGACCAAGCA | Template to |
| 166 | GTATCAGGCGCTGTTTAATCTGGAAGGCGTGGATCTGGAATTCCG | amplify lon |
| | TGACGAGGCGCTGGATGCTATCGCTAAGAAAGCGATGGCGCGTAA | popout |
| | AACCGGTGCCCGTGGCCTGCGTTCCATCGTAGAAGCCGCACTGCT | cassette |
| | CGATACCATGTACGATCTGCCGTCCATGGAAGACGTCGAAAAAGT | |
| | GGTTATCGACGAGTCGGTAATTGATGGTCAAAGCAAACCGTTGCT | |
| | GATTTATGGCAAGCCGGAAGCGCAACAGGCATCTGGTGAATAATT | |
| | AACCATTCCCATACAATTAGTTAACCAAAAAGGGGGGATTTTATC | |
| | TCCCCTTTAATTTTTCCTCTATTCTCGGCGTTGAATGTGGGGGAA | |
| | ACATCCCCATATACTGACGTACATGTTAATAGATGGCGTGAAGCA | |
| | CAGTCGTGTCATCTGATTACCTGGCGGAAATTAAACTAAGAGAGA | |
| | GCTCTTGACCTCGCGCAAAATGCACTAATAAAAACAGGGCTGGCA | |
| | GGCTAATTCGGGCTTGCCAGCCTTTTTTTGTCTCGCTAAGTTAGA | |
| | TGGCGGATCGGGCTTGCCCTTATTAAGGGGTGTTGTAAGGGGATG | |
| | GCTGGCCTGATATAACTGCTGCGCGTTCGTACCTTGAAGGATTCA | |
| | AGTGCGATATAAATTATAAAGAGGAAGAGAAGAGTGAATAAATCT | |
| | CAATTGATCGACAAGATTGCTGCAGGGGCTGATATCTCTAAAGCT | |
| | GCGGCTGGCCGTGCGTTAGATGCTATTATTGCTTCCGTAACTGAA | |
| | TCTCTGAAAGAAGGGGATGATGTAGCACTGGTAGGTTTTGGTACT | |
| | TTTGCCGTTAAAGAGCGTGCTGCCCGTACTGGCCGCAACCCGCAG | |
| | ACCGGTAAAGAGATCACCATCGCTGCTGCTAAAGTACCGAGCTTC | |
| | CGTGCAGGTAAAGCACTGAAAGACGCGGTAAACTAAGCGTTGTCC | |
| | CCAGTGGGGA (SEQ ID NO: 25) | |

### Construction of Knockout Cassettes

Knockout cassettes for strain engineering work included a DNA cassette than encodes a kanamycin resistance marker (kan) in addition to sacB (encoding the enzyme levansucrase) for negative selection. To allow for the integration of this kan-sacB knockout cassette into the correct location of the genome, small 45-bp upstream and downstream homologous regions (UHR and DHR, respectively) were appended onto the knockout cassette using PCR (FIG. 1) and Invitrogen Platinum SuperFi PCR Master Mix. The knockout cassette was amplified from an internally produced plasmid containing the kan-sacB expression cassette, pStrain 22.

Introduction of Scar-Less Genomic Deletions in E. coli

The strain to be genetically modified was first transformed with pStrain 21 and transformants selected for by plating onto LB-animal free (LBAF) agar containing 100 μg/ml carbenicillin. A single transformant was then grown up in LBAF broth containing 100 μg/ml carbenicillin @ 30° C. for 16 hours followed by transferring 30 μl of this overnight culture into a test tube containing 3 ml LBAF broth with 100 μg/ml carbenicillin and incubated for 2 hours at 30° C., 250 rpm. After 2 hours of incubation, expression of the genes encoding the lambda red system and a codon optimized E. coli recA were induced using 100 ng/ml anhydrotetracycline and 1 mM isopropyl β-D-1-thiogalactopyranoside, respectively. After 2-3 additional hours of shaking incubation at 30° C., when OD600 ˜0.6-1.0, 1 ml of culture was harvested to prepare 0.1 ml electrocompetent cells. 50 μl of electrocompetent cells were mixed with 1 μg of purified knockout cassette and electroporated in 1 mm gapped cuvettes @ 1800 volts. Transformations were rescued in 1 ml SOC media @ 30° C., 300 rpm for 2 hr then plated onto LBAF agar containing 50 μg/ml kanamycin and 100 μg/ml carbenicillin and incubated overnight at 30° C. Colony PCR (cPCR) with LongAmp Taq DNA polymerase was then utilized to screen for primary integrants using a universal primer that binds to the kanamycin resistance gene, kan, and a location-specific primer that binds upstream of the gene targeted for knockout. In parallel with cPCR, the same clones were spotted onto LBAF agar containing 35 μg/ml kanamycin and 100 μg/ml carbenicillin and LB agar containing 60 g/l sucrose. These plates are incubated overnight at 30° C. After confirmation of primary integrants by cPCR, the sucrose sensitivity that was expected was confirmed by visually checking for a “no growth” phenotype where the clone was spotted onto LBAF agar containing 60 g/l sucrose. Once a primary integration clone was confirmed by cPCR and was also confirmed to be sucrose-sensitive, the knockout cassette was removed using a similar approach as described below.

To remove a given knockout cassette and obtain a scar-less deletion, a linear dsDNA fragment containing only the UHR and DHR regions was amplified from gBlocks (IDT) and unique sets of primers as indicated in Table 1. For convenience, these linear dsDNA fragments used to remove the kan-sacB cassettes are called ‘popout cassettes’. Confirmed primary integrants were grown up in LBAF broth containing 100 μg/ml carbenicillin and 50 μg/ml kanamycin @ 30° C. for 16 hours followed by transferring 30 μl of this overnight culture into a test tube containing 3 ml LBAF broth with 100 μg/ml carbenicillin and 50 μg/ml kanamycin and incubated for 2 hours at 30° C. 250 rpm. After 2 hours of incubation, expression of the genes encoding the lambda red system and a codon optimized E. coli recA were induced using 100 ng/ml aTc and 1 mM IPTG, respectively. After 2-3 additional hours of shaking incubation at 30° C., when OD600 ˜0.6-1.0, 1 ml of culture was harvested to prepare 0.1 ml electrocompetent cells. 50 μl of electrocompetent cells were mixed with 1 μg of purified popout cassette, and electroporated in 1 mm gapped cuvettes @ 1800 volts. Transformations were rescued in 1 ml SOC media @ 30° C. 300 rpm for 2 hr then transferred to a 125 ml shake flask containing 9 ml LBAF broth. This diluted culture was then grown at 30° C. 300 rpm for 5-16 hours followed by transferring 50 μl of culture into a test tube containing 5 ml LBAF-no salt broth containing sucrose (10 g/l soytone, 5 g/l yeast extract, 60 g/l sucrose; filter sterilized with 0.2 uM filter). This sucrose-containing culture was then incubated at 30° C. 250 rpm overnight (˜16 hours). Grown up culture was then diluted 1 million-fold in sterile LBAF broth, plated onto LBAF agar (200 μl plated) and incubated overnight at 37° C.

Once isolated colonies were obtained on LBAF agar plate, clones were screened for successful removal of the knockout cassette (kan-sacB) using cPCR and primers that bind upstream and downstream of the gene(s) to be knocked out. In parallel, the clones were replica-spotted onto LBAF agar and LBAF agar containing 100 μg/ml carbenicillin. These plates were incubated overnight (16 hours) at 30° C. to confirm loss of the temperature-sensitive plasmid needed for genome editing, pStrain 21. DNA sequences of the linear popout cassettes used to construct Strain 11 and Strain 17 are listed in the Exemplary Sequences table.

### Strain Confirmation of Strain 17

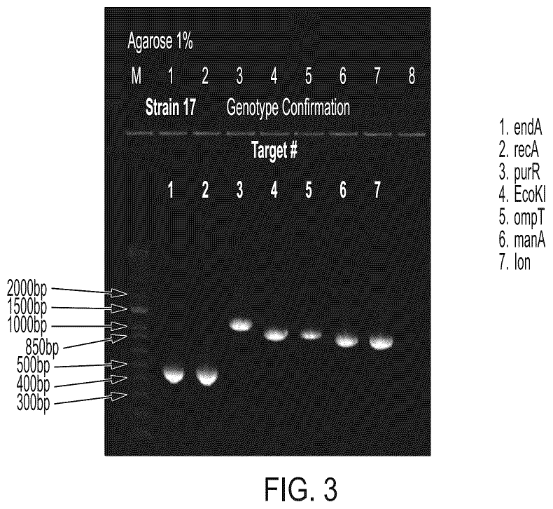

PCR was performed to confirm the genotype of Strain 17. Template DNA was isolated using shavings from the Strain 17 glycerol stock. PCR reactions were setup using the appropriate primers (Table 3) for each target gene and LongAmp Taq DNA polymerase (Cat #M0323S). After analyzing the gel in FIG. 3 all banding patterns were consistent with expected molecular weights at the edited loci. Also, the PCR products were sequence confirmed to verify that the expected mutations were introduced at the desired loci.

TABLE 5
Expected PCR amplicon size for various genomic loci in parental
strain Strain 2 and custom protein expression strain, Strain 17,
using indicated primers (see Table 3 for primer sequences).

| | | | Expected | Expected |
|---|---|---|---|---|
| | | | PCR | PCR |
| | | | product | product |
| | | | length | length |
| | | | from | from |
| Target genomic locus for | Forward | Reverse | Strain 2 | Strain 17 |
| identity verification by PCR | primer | primer | (bp) | (bp) |
| endA | KS518 | KS519 | 499 | 499 |
| recA | KS520 | KS521 | 500 | 500 |
| purR | KS1431 | KS1432 | 1226 | 1226 |
| mrr-hsdRMS-symE-mcrBC | KS1112 | KS1113 | 1048 | 1048 |
| (EcoKI) | | | | |
| ompT | KS651 | KS652 | 1954 | 955 |
| manA | KS1729 | KS1730 | 2162 | 986 |
| lon | KS979 | KS980 | 3330 | 843 |

### Results

T7 RNA Polymerase Variant 1 Expression with Strain 11

Strain 11/pStrain 2 and BL21/pStrain 2 clones were incubated overnight at 30° C., 300 rpm. The following day baffled 500 mL shake flasks were prepared containing 60 ml TBAF++ and 50 μg/ml kanamycin. A portion of each overnight culture was transferred to a flask to obtain initial OD600=0.05. Flasks were then incubated at 28° C., 300 rpm for the duration of the experiment. Flasks were induced with 1 mM IPTG at hour 12 (roughly OD600=15). Every hour post-induction, OD600 was measured and 1 OD600-equivalent cell pellets were saved for lysis and protein gels at −20° C.

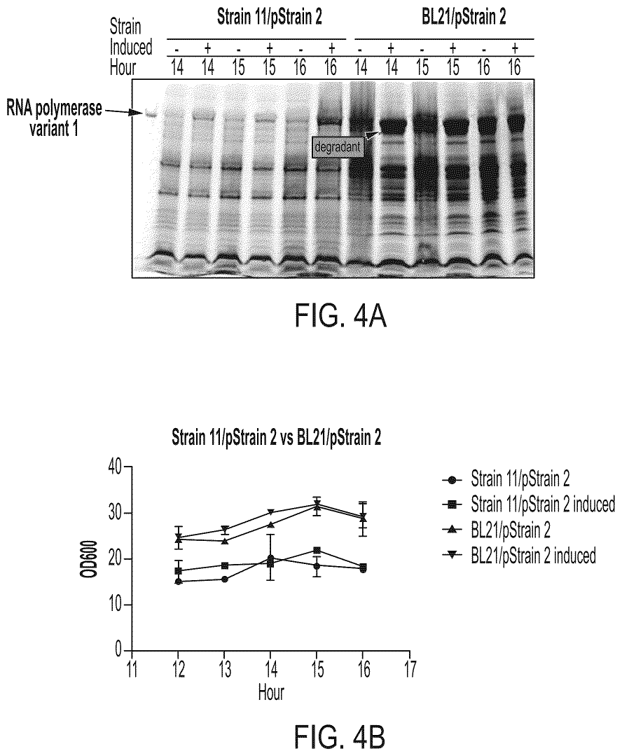

After successfully creating Strain 11, it was transformed with plasmid pStrain 2, creating Strain 12, to allow for high-copy, IPTG-inducible expression of T7 RNA polymerase variant 1. The growth profile, characteristics, yield and purity of T7 RNA polymerase variant 1 protein of Strain 12 was initially assayed in shake flasks as described in method section. As a comparison, BL21 carrying pStrain 2 was also included in the shake flask study. As shown in FIG. 4, growth of Strain 12 and expression of the T7 RNA polymerase variant lenzyme is comparable to the BL21. Also, BL21 showed displayed a degradant of T7 RNA polymerase variant 1 that was not observed in Strain 11. Unfortunately, Strain 11 displayed a negative growth phenotype resulting in a very viscous, slurry-like culture, which lowered the mixing performance of Ambr bioreactors, resulting in reduced oxygen mass transfer and reduced ability to maintain target % dissolved oxygen (data not shown).

The mucoid phenotype appeared to be the result of the lon deletion as the ompT knockout by itself in Strain 10 had normal growth and culture appearance (data not shown). Without being bound by theory, it is possible that the complete removal of Lon causes overaccumulation of RcsA, therefore resulting in overproduction of a polysaccharide called colanic acid. Thus, manA, a gene that encodes for the first step in the mannose biosynthesis pathway, was deleted from the genome. This non-essential gene was deleted from the genome to eliminate the overproduction of polysaccharide and thereby abrogate the mucoid phenotype.

manA Knockout Results in Loss of Undesirable Mucoid Phenotype

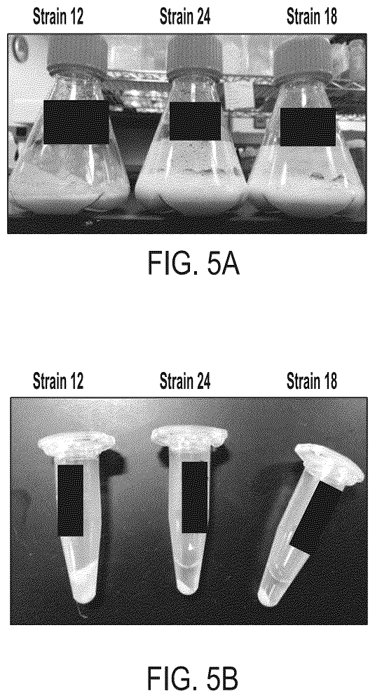

After having constructed Strain 17, it was demonstrated that the manA knockout removes the mucoid phenotype that arises from the necessary lon knockout. Strain 11, BL21 and Strain 17 were transformed with pStrain 2 (for expression of T7 RNA polymerase variant 1) to obtain Strain 12, Strain 24, and Strain 18, respectively. As can be clearly seen from FIG. 5A, Strain 12 (Strain 11/pStrain 2) displays a dense mucoid formation, evident by lack of frothing and trapped bubbles. On the other hand, strains Strain 24 and Strain 18 (host strains BL21 and Strain 17, respectively) display normal phenotypes with the T7 RNA polymerase variant 1-encodingplasmid present. To further demonstrate the improvement in cell morphology and its utility in industrial processes, Strain 12, Strain 24, and Strain 18 cultures were pelleted by centrifugation @ 13.000×g. As shown in FIG. 5B. Strain 24 (BL21/pStrain 2) created dense pellets. However, Strain 12 (Strain 11/pStrain 2), which has manA intact, did not pellet well. But, Strain 18 (Strain 17/pStrain 2), the manA knockout strain, created crisp, dense pellets with clear supernatant. These results from shake flask fermentations supported further testing of Strain 17 for recombinant protein expression in shake flasks and Ambr250 bioreactors.

Expression of T7 RNA Polymerase Variant 1 with Host Strain Strain 17

To perform shake flask expression fermentations, Strain 17/pStrain 2 clones were inoculated into 3 ml LBAF with 50 μg/ml kanamycin test tubes and incubated overnight 30° C. at 300 rpm. The OD600 of each culture was determined and the necessary amount of each seed culture to obtain initial OD600=0.1 was transferred to sterile 250 ml baffled, vented shake flasks containing 30 ml TBAF+50 μg/ml kanamycin. Inoculated flasks were then incubated at 37° C., 300 rpm. At 3 hr timepoint, 0.5 mM IPTG was added to induce T7 RNA polymerase variant 1 expression. OD600 and 1 OD-equivalent cell pellets were taken 4 hours post-induction.

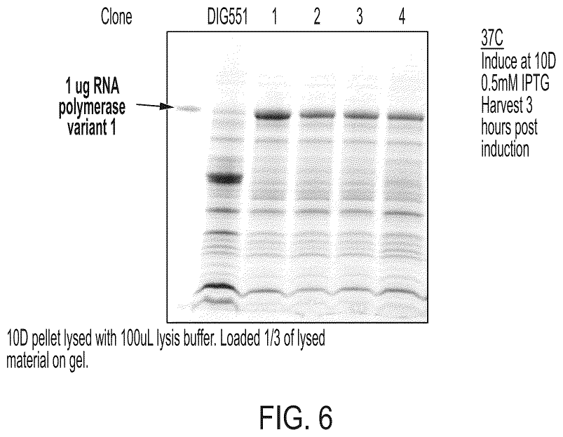

This study evaluated four different clones expressing T7 RNA polymerase variant 1 in Strain 17. In FIG. 6, overexpression of T7 RNA polymerase variant 1 was observed in all clones while clone 1 appeared to have the highest expression of T7 RNA polymerase variant 1. Possibly due to the lack of the mucoid phenotype, lysis was more efficient resulting in a clean gel image as compared to experiments using Strain 11 as the host strain.

### Performance of Strain 17 Expressing T7 RNA Polymerase Variant 2 in Ambr250 Bioreactors

After Strain 17 was transformed with pStrain 23, which carries an IPTG-inducible DNA sequence encoding T7 RNA polymerase variant 2, two clones were picked for further screening and characterization: Strain 19 and Strain 20. Once the clones' plasmid and genotypes were confirmed, their performance in an AMBR250 bioreactor was evaluated. This process involves aerobic fed-batch fermentations using TBAF media, yeastolate, and glycerol feeds.

For this experiment, one 500 mL shake flask was inoculated using an ice shaving from glycerol stock to 75 mL of TBAF+50 μg/mL kanamycin and grown for 9.5 hours at 28° C. Each bioreactor was inoculated with 6.7% inoculum (initial OD₆₀₀ 0.05), which was calculated based on the initial starting volume of 200 mL and then multiplied by the seed flask target OD600 of 0.8. This value was then normalized by the actual seed flask OD600.

TABLE 6
Wet cell weights from 28 hr EFT timepoint.

| | | Wet cell |
|---|---|---|
| Reactor # | Clone | weight (g/L) |
| 1 | Strain 19 | 163 |
| 2 | Strain 19 | 151 |
| 3 | Strain 20 | 148 |
| 4 | Strain 20 | 157 |

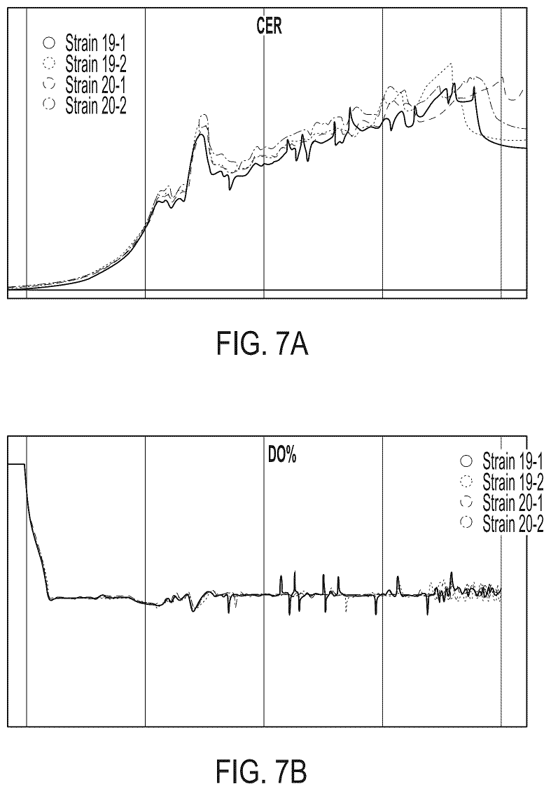

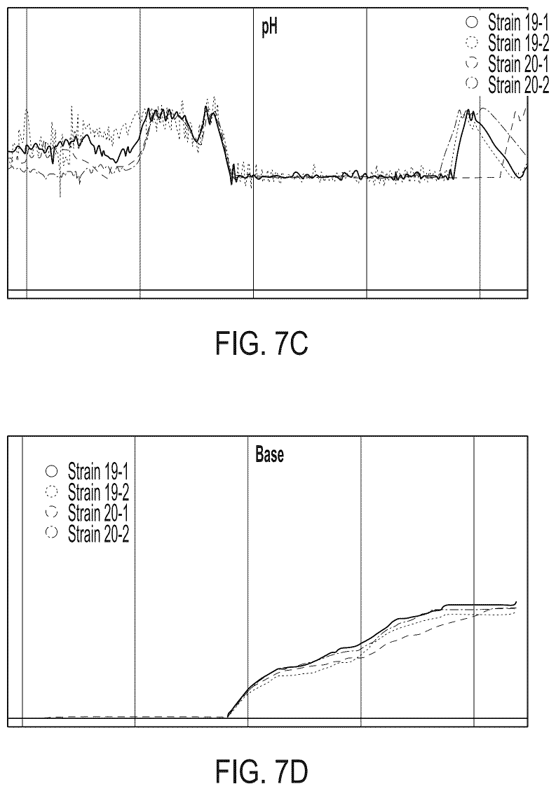

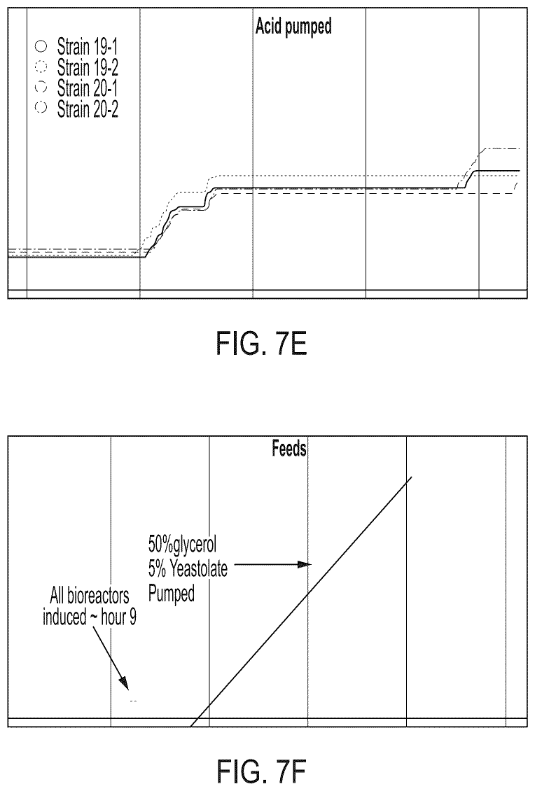

Online profiles aligned with expectations and were consistent between all bioreactors (FIG. 7). CER reached a value of 60 at hour 9, triggering induction of all four bioreactors with IPTG bolus addition. CER continued to climb throughout the culture duration reaching a peak CER of 90. Dissolved Oxygen was maintained at 50% for the entire culture duration with only minor fluctuations that are typical for these sensors. pH was maintained at a target of 6.95±0.05. The pH climbed above 7.0 during exponential phase triggering acid additions until the pH stabilized around hour 11. Once the pH stopped climbing and began to stabilize at hour 11, base was triggered to feed at a near constant rate to maintain a pH of 6.95. At 28 hours, the culture was harvested and sent for purification and LabChip analysis (protein quantification by capillary electrophoresis). Both clones, Strain 19 and Strain 20, behaved similarly, grew to similar final wet cell weights (Table 6) and, importantly, no issues were observed using host strain Strain 17 for recombinant protein expression in high density bioreactor fermentations.

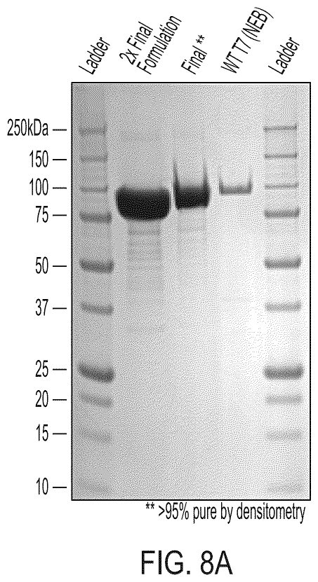

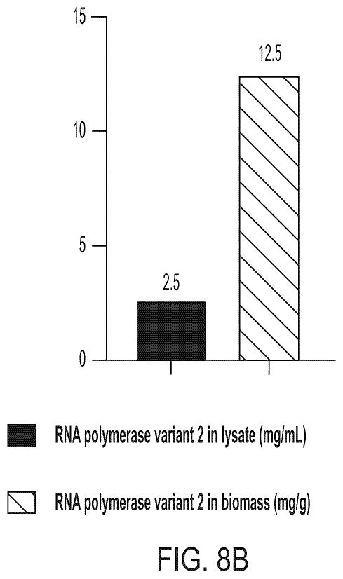

Finally, T7 RNA polymerase variant 2 expression was visualized, quantified and purified from frozen cell pellets taken during the Ambr fermentation described above. Cell pellets from Strain 19 bioreactors were processed by freezing at −80° C., thawing to ambient temperature, lysing bacterial cells in the presence of Tris-HCl, NaCl, DTT, protease inhibitors, PMSF, lysozyme. DNAse, Triton-X 100. Lysate was filtered, and the T7 RNA polymerase variant 2 present in the filtrate was analyzed by capillary electrophoresis. Results are shown in FIG. 8. The resulting final protein yield was approximately 2.5 g/L (grams of protein per liter of culture) with purity >95% purity via densitometry. The T7 RNA polymerase variant 2 yield achieved here is quite high for a 1st generation strain and fermentation process and, if successfully scaled to a 30 L fermentation, is expected to deliver >60 grams of unpurified T7 enzyme per fermentation batch. For context, production of 100-150 g of mRNA requires approximately 1 g of T7 RNA polymerase.

### CONCLUSIONS

A custom E. coli strain for expression of recombinant protein has been developed. To begin, Strain 2 was selected as the parent strain to begin introducing genomic modifications that are specifically beneficial for recombinant protein expression. Strain 2 possesses several mutations that are useful for an industrial protein expression strain. These include endA and recA knockouts to increase plasmid stability and purity upon purification, and inactivation of the EcoKI restriction system for improved transformation and cloning efficiency. An aim of this work was to decrease protease activity of Strain 2 to deliver a strain that can express recombinant enzymes with high purity.

The ompT gene, which encodes a major protease that is found in the outer membrane of E. coli, was deleted from the genome. Secondly, a major house-keeping protease Lon was deleted from the genome. It was discovered that a lon knockout in the strain resulted in a negative mucoid phenotype, caused by upregulation of a polysaccharide synthesis pathway. This lon knockout exhibited poor performance in Ambr250 bioreactors as the culture viscosity became a limitation on the agitation and aeration of the culture. According to the genome sequence of E coli BL21 (Genbank: CP053601.1), the lon ORF remains intact in this strain. It is possible BL21 does exhibit some residual Lon activity and, therefore, does not display a mucoid phenotype. To prevent degradation of a desired recombinant protein, a strain entirely deficient in Lon activity. To develop a strain that is a true lon knockout, but that also does not exhibit a mucoid phenotype, an additional gene knockout ΔmanA, was introduced to remove the 1st step of a non-essential polysaccharide synthesis pathway. The final genotype of the 1st generation protein expression strain, Strain 17, is E. coli MG1655 ΔendA ΔrecA Δ(mrr-hsdRMS-symE-mcrBC) ΔompT ΔmanA Δlon.

### Example 2: Production of T7 RNA Polymerase Variant 3 and T7 RNA Polymerase Variant 3 in Genetically Modified E. coli Strain Deficient in Lon, OmpT, and ManA

pStrain 23, encoding an IPTG-inducible DNA sequence encoding T7 RNA polymerase variant 2, is modified by site-directed mutagenesis so that the coding sequence instead encodes T7 RNA polymerase variant 3. This modified plasmid is referred to as pStrain 24. Strain 17 is transformed with pStrain 24, which carries an IPTG-inducible DNA sequence encoding T7 RNA polymerase variant 3. Once the genotypes of the bacterial strains and transformed plasmids are confirmed, the strains are inoculated into an AMBR250 bioreactor to initiate fermentation and production of T7 RNA polymerase variant 3. This process involves aerobic fed-batch fermentations using TBAF media, yeastolate, and glycerol feeds.

IPTG is added to each bioreactor to trigger induction of T7 RNA polymerase variant 3 expression. The bacterial culture is grown, and then the culture is harvested and sent for purification of T7 RNA polymerase variant 3 and subsequent LabChip analysis (protein quantification by capillary electrophoresis).

Finally, T7 RNA polymerase variant 3 expression is visualized, quantified and purified from frozen cell pellets taken during the Ambr fermentation described above. Cell pellets from Strain 19 bioreactors are processed by freezing at −80° C., thawing to ambient temperature, lysing bacterial cells in the presence of Tris-HCl, NaCl, DTT, protease inhibitors, PMSF, lysozyme, DNAse, Triton-X 100. Lysate is filtered, and the T7 RNA polymerase variant 3 present in the filtrate is analyzed by capillary electrophoresis. If scaled to a 30 L fermentation, fermentation is expected to deliver >60 grams of unpurified T7 RNA polymerase variant 3 enzyme per fermentation batch. For context, production of 100-150 g of mRNA requires approximately 1 g of T7 RNA polymerase.

EXEMPLARY SEQUENCES

| SEQ | | |
|---|---|---|
| ID | | |
| NO | SEQUENCE | DESCRIPTION |
| 26 | TATTGAAGATCCGCTTCGATCAGGGGATGGGCTACTGGCGCATCAACTTTTCATCGC | mcrC |
| | AATGGCATTACTTTGATTTCCGCGATGACGTTTCTTTCCAGTTAGTCAAAATGGCTC | deletion |
| | AGGCCTGCAAGGAAGGGAATGTCGCCAACAGCGAAGAGAGTTGGGCAACGGATGTGC | cassette |
| | TGGTGGAGGTGATCGCCTCCTGATGATGAGCCGCTCCCGATGTGGTGTCGGGAGCGG | |
| | TATTTTCTATAAAACTTACCGCAATATCAGGCCGGATGCGGCTGCGCCTTATCCGGC | |
| | CCATAACCCCTTACTTCCTCAACCCCGCAAACGCAGCCCGAATCTCTTCCTCCGGCA | |
| | GCTGGATCCCGATAAACACCATCGTGCTATGCGGTTTTTCATCGCCCCACGGCCTGT | |
| | CCCAGTCGGCGCTGTAGAGGCGCTGGACGCCCTGGAACAGCAGGCGGTTAGGTTCGC | |
| | CGTCAATCCACAGCATCCCTTTGTAACGTAGCAGTTTATCCGCC | |
| 23 | AACGGATAAGACGGGCATAAATGAGGAAGAAATGGCGCGCCCTGCAGGATTCGAACC | ompT |
| | TGCGGCCCACGACTTAGAAGTTCCTAGAACGACATTTTAAGTCAACAACTTACCGCG | deletion |
| | CCATCTCTGCGCTCACACGTCCCACTACCTCAAAACATGTAAAGCCTTGCAAGCCAT | cassette |
| | TGCGAGGCCTTATGTGTCTCAGTTTTGTCCCTCTTTTTTGTACTAAAAAACATAGTA | |
| | ATTGAGGATAAACCTCATGCTATTTTCGCTTATATGCCTCTAAAGGCATGGCACTTA | |
| | AATAGATAAAAGCACCACAAAAGCATAAAAAAACCACACAGTAAAACCGAAATATGA | |
| | AACAATAACAGATAATTAAACCAAAAACAGATAGCGCATTGTGATAATCATTCAATA | |
| | CTAAACAAAATATAAACAGTGGAGCAATATGTAATTGACTCATTAAGTTAGATATAA | |
| | AAAATACATATTCAATCATTAAAACGATTGAATGGAGAACTTTTGAACGCCAACTAA | |
| | AATTTCCCCGAGGTGAAAATCGCCCCGGGGAATAACTAGCCATTTCAATGTAACAAT | |
| | TAACCCTTAAAATAAACCCAGAAGGTTATTAACTAAATCACATAGAAAACCATCAAT | |
| | TATAGTATGTATAAAATAGGCGACAGCAACCCAATTACAAATTAATGGTTCCAGAAT | |
| | ATCACATCAAAAAAAACGCTGTATAATATTATAATTAACATGTAGACAACTTGTAAT | |
| | AAACATTATCAGTCAATTGTTTTGTTTATTCCATCTGTGACGCCGATTATTTTCTCA | |
| | AAATAATGAGATGGCGTGACACCATAATAATCTTTAAATGCACATATGAAATATGAA | |
| | GTACTGTTATAGCCACATTTCTGGGCTACGACATTGATAGAATAAGAGTTTGAAGTT | |
| | ATGAGTTTTTTTGCATACCTCATCCTAGTATCTCTCAATATTTCAGTAAATGACGTT | |
| | CCTTCATCCCTTAATCTTTTTTTTATTAAAC | |
| 25 | TCTGATTCAGATCCTCAAAGAGCCGAAAAACGCCCTGACCAAGCAGTATCAGGCGCT | lon |
| | GTTTAATCTGGAAGGCGTGGATCTGGAATTCCGTGACGAGGCGCTGGATGCTATCGC | deletion |
| | TAAGAAAGCGATGGCGCGTAAAACCGGTGCCCGTGGCCTGCGTTCCATCGTAGAAGC | cassette |
| | CGCACTGCTCGATACCATGTACGATCTGCCGTCCATGGAAGACGTCGAAAAAGTGGT | |
| | TATCGACGAGTCGGTAATTGATGGTCAAAGCAAACCGTTGCTGATTTATGGCAAGCC | |
| | GGAAGCGCAACAGGCATCTGGTGAATAATTAACCATTCCCATACAATTAGTTAACCA | |
| | AAAAGGGGGGATTTTATCTCCCCTTTAATTTTTCCTCTATTCTCGGCGTTGAATGTG | |
| | GGGGAAACATCCCCATATACTGACGTACATGTTAATAGATGGCGTGAAGCACAGTCG | |
| | TGTCATCTGATTACCTGGCGGAAATTAAACTAAGAGAGAGCTCTTGACCTCGCGCAA | |
| | AATGCACTAATAAAAACAGGGCTGGCAGGCTAATTCGGGCTTGCCAGCCTTTTTTTG | |
| | TCTCGCTAAGTTAGATGGCGGATCGGGCTTGCCCTTATTAAGGGGTGTTGTAAGGGG | |
| | ATGGCTGGCCTGATATAACTGCTGCGCGTTCGTACCTTGAAGGATTCAAGTGCGATA | |
| | TAAATTATAAAGAGGAAGAGAAGAGTGAATAAATCTCAATTGATCGACAAGATTGCT | |
| | GCAGGGGCTGATATCTCTAAAGCTGCGGCTGGCCGTGCGTTAGATGCTATTATTGCT | |
| | TCCGTAACTGAATCTCTGAAAGAAGGGGATGATGTAGCACTGGTAGGTTTTGGTACT | |
| | TTTGCCGTTAAAGAGCGTGCTGCCCGTACTGGCCGCAACCCGCAGACCGGTAAAGAG | |
| | ATCACCATCGCTGCTGCTAAAGTACCGAGCTTCCGTGCAGGTAAAGCACTGAAAGAC | |
| | GCGGTAAACTAAGCGTTGTCCCCAGTGGGGA | |
| 24 | ACGCCTTTCGCCGCGATGTCGGAGTTACGCAGGAATTGCAGCGCCACATATTTATCA | manA |
| | TTTTCGCTGGCCTCCGGGTCACGCAGAATGTCGGCCACCTGTTGTTGGTGCGCCGGA | deletion |
| | CGCAGCATGAACGACGCATCATGAAACGCCTGGCGCGCCAACAGAGTTAACGCTTCG | cassette |
| | GGTGCGACTTTCAAAATCTCCTGCCCTTCAAATTCAGATACGCTAACGTGTTCGCTG | |
| | GTTAGCAGGTAATACTCAGTATCATCTTTTTTGAGTGGAAAAGGAGCCTGATAATGA | |
| | AAGGGTTTGTTTGACATTGTTCTCTCACTTACTGCCTGGTTTGGTTATGCTCTGGGC | |
| | GGGTGTTCCGTTGCCCTGTTAAAAGCGAGTAACAATATCCTACACACTTTTTTAACA | |
| | AAAACTGAGACTAGTACGACTTTTTGCGGCTCCAGGTTACTTCCCGTAGGATTCTTG | |
| | CTTTAATAGTGGGATTAATTTCCACATTAAAACAGGGATTGATCGAGCTTACTGAAA | |
| | AAATTAACATCTCTTGCTAAGCTTATTAAAGGCTTATAACACCTTCAGGCGGCCAGT | |
| | CCGCCTGATTTCATTTTATGGATAATCATTATGAATAAATCGCTGGTAGCGGTAGGC | |
| | GTCATTGTTGCGCTAGGCGTAGTCTGGACAGGCGGCGCATGGTATACAGGCAAGAAG | |
| | ATTGAAACCCATCTCGAAGACATGGTCGCGCAGGCGAACGCGCAACTCAAACTGACA | |
| | GCTCCTGAATCCAACCTGGAAGTGAGTTATCAAAACTATCATCGCGGCGTATTCAGC | |
| | AGCCAGTTGCAACTGTTGGTGAAACCCATTGCCGGGAAAGAAAATCCGTGGATTAAA | |
| | AGCGGTCAGAGCGTCATCTTCAACGAATCGGTTGATCATGGTCCCTTCCCGCTTGCC | |
| | CAGCTTAAAAAACTGAACCTGATCCCGTCGATGGCATCAATTCAAACCACGCTGGTT | |
| | AATAACGAAGTAAGCAAACCACTGTTTGATA | |
| 27 | GATTCGAACTCGGCCCACGACTTAGAAGTTCTAGAACGACATTTTAAGTCAACAACT | ompT DNA |
| | TACCGCGCCATCTCTGCGCTCACACGTCCCACTACCTCAAAACATGTAAAGCCTTGC | coding |
| | AAGCCATTGCGAGGCCTTATGTGTCTCAGTTTTGTCCCTCTTTTTTGTACTAAAAAA | sequence |
| | CATAGTAATTGAGGATAAACCTCATGCTATTTTCGCTTATATGCCTCTAAAGGCATG | |
| | GCACTTAAATAGATAAAAGCACCACAAAAGCATAAAAAAACCACACAGTAAAACCGA | |
| | AATATGAAACAATAACAGATAATTAAACCAAAAACAGATAGCGCATTGTGATAATCA | |
| | TTCAATACTAAACAAAATATAAACAGTGGAGCAATATGTAATTGACTCATTAAGTTA | |
| | GATATAAAAAATACATATTCAATCATTAAAACGATTGAATGGAGAACTTTTATGCGG | |
| | GCGAAACTTCTGGGAATAGTCCTGACAACCCCTATTGCGATCAGCTCTTTTGCTTCT | |
| | ACCGAGACTTTATCGTTTACTCCTGACAACATAAATGCGGACATTAGTCTTGGAACT | |
| | CTGAGCGGAAAAACAAAAGAGCGTGTTTATCTAGCCGAAGAAGGAGGCCGAAAAGTC | |
| | AGTCAACTCGACTGGAAATTCAATAACGCTGCAATTATTAAAGGIGCAATTAATTGG | |
| | GATTTGATGCCCCAGATATCTATCGGGGCTGCTGGCTGGACAACTCTCGGCAGCCGA | |
| | GGTGGCAATATGGTCGATCAGGACTGGATGGATTCCAGTAACCCCGGAACCTGGACG | |
| | GATGAAAGTAGACACCCTGATACACAACTCAATTATGCCAACGAATTTGATCTGAAT | |
| | ATCAAAGGCTGGCTCCTCAACGAACCCAATTACCGCCTGGGACTCATGGCCGGATAT | |
| | CAGGAAAGCCGTTATAGCTTTACAGCCAGAGGTGGTTCCTATATCTACAGTTCTGAG | |
| | GAGGGATTCAGAGATGATATCGGCTCCTTCCCGAATGGAGAAAGAGCAATCGGCTAC | |
| | AAACAACGTTTTAAAATGCCCTACATTGGCTTGACTGGAAGTTATCGTTATGAAGAT | |
| | TTTGAACTCGGTGGCACATTTAAATACAGCGGCTGGGTGGAATCATCTGATAACGAT | |
| | GAACACTATGACCCGGGAAAAAGAATCACTTATCGCAGTAAGGTCAAAGACCAAAAT | |
| | TACTATTCTGTTGCAGTCAATGCAGGTTATTACGTCACACCTAACGCAAAAGTTTAT | |
| | GTTGAAGGCGCATGGAATCGGGTTACGAATAAAAAAGGTAATACTTCACTTTATGAT | |
| | CACAATAATAACACTTCAGACTACAGCAAAAATGGAGCAGGTATAGAAAACTATAAC | |
| | TTCATCACTACTGCTGGTCTTAAGTACACATTTTAAGAACGCCAACTAAAATTTCCC | |
| | CGAGGTGAAAATCGCCCCGGGAATAACTAGCCATTTCAATGTAACAATTAACCCTTA | |
| | AAATAAACCCAGAAGGTTATTAACTAAATCACATAGAAAACCATCAATTATAGTATG | |
| | TATAAAATAGGCGACAGCAACCCAATTACAAATTAATGGTTCCAGAATATCACATCA | |
| | AAAAAAACGCTGTATAATATTATAATTAACATGTAGACAACTTGTAATAAACATTAT | |
| | CAGTCAATTGTTTTGTTTATTCCATCTGTGACGCCGATTATTTTCTCAAAATAATGA | |
| | GATGGCGTGAGACCATAATAATCTTTAAATGCACATATGAAATATGAAG | |
| 28 | MRAKLLGIVLTTPIAISSFASTETLSFTPDNINADISLGTLSGKTKERVYLAEEGGR | OmpT amino |
| | KVSQLDWKENNAAIIKGAINWDLMPQISIGAAGWTTLGSRGGNMVDQDWMDSSNPGT | acid |
| | WTDESRHPDTQLNYANEFDLNIKGWLLNEPNYRLGLMAGYQESRYSFTARGGSYIYS | sequence |
| | SEEGFRDDIGSFPNGERAIGYKQRFKMPYIGLIGSYRYEDFELGGTFKYSGWVESSD | |
| | NDEHYDPGKRITYRSKVKDQNYYSVAVNAGYYVTPNAKVYVEGAWNRVINKKGNTSL | |
| | YDHNNNTSDYSKNGAGIENYNFITTAGLKYTF | |
| 29 | GAATTCCGTGACGAGGCGCTGGATGCTATCGCTAAGAAAGCGATGGCGCGTAAAACC | lon DNA |
| | GGTGCCCGTGGCCTGCGTTCCATCGTAGAAGCCGCACTGCTCGATACCATGTACGAT | coding |
| | CTGCCGTCCATGGAAGATGTCGAAAAAGTGGTTATCGACGAGTCGGTAATTGATGGT | sequence |
| | CAAAGCAAACCGTTGCTGATTTATGGTAAGCCGGAAGCGCAACAGGCATCTGGTGAA | |
| | TAATTAACCATTCCCATACAATTAGTTAACCAAAAAGGGGGGATTTTATCTCCCCTT | |
| | TAATTTTTCCTCTATTCTCGGCGTTGAATGTGGGGGAAACATCCCCATATACTGACG | |
| | TACATGTTAATAGATGGCGTGAAGCACAGTCGTGTCATCTGATTACCTGGCGGAAAT | |
| | TAAACTAAGAGAGAGCTCTATGAATCCTGAGCGTTCTGAACGCATTGAAATCCCCGT | |
| | ATTGCCGCTGCGCGATGTGGTGGTTTATCCGCACATGGTCATCCCCTTATTTGTCGG | |
| | GCGGGAAAAATCTATCCGTTGTCTGGAAGCGGCGATGGACCATGATAAAAAAATTAT | |
| | GCTGGTCGCGCAGAAAGAAGCTTCAACGGATGAGCCGGGTGTAAACGATCTTTTCAC | |
| | CGTCGGGACCGTGGCCTCTATATTGCAGATGCTGAAACTGCCTGACGGCACCGTCAA | |
| | AGTGCTGGTCGAGGGGTTACAGCGCGCGCGTATTTCTGCGCTCTCTGACAATGGCGA | |
| | ACACTTTTCTGCGAAGGCGGAGTATCTGGAGTCGCCGACCATTGATGAGCGGGAACA | |
| | GGAAGTGCTGGTGCGTACTGCAATCAGCCAGTTCGAAGGCTACATCAAGCTGAACAA | |
| | AAAAATCCCACCAGAAGTGCTGACGTCGCTGAATAGCATCGACGATCCGGCGCGTCT | |
| | GGCGGATACCATTGCTGCACATATGCCGCTGAAACTGGCTGACAAACAGTCCGTTCT | |
| | GGAGATGTCCGACGTTAACGAACGTCTGGAATATCTGATGGCAATGATGGAATCGGA | |
| | AATCGATCTGCTGCAGGTTGAGAAACGCATTCGCAACCGCGTTAAAAAGCAGATGGA | |
| | GAAATCCCAGCGTGAGTACTATCTGAACGAGCAAATGAAAGCTATTCAGAAAGAACT | |
| | CGGTGAAATGGACGACGCGCCGGACGAAAACGAAGCCCTGAAGCGCAAAATCGACGC | |
| | GGCGAAGATGCCGAAAGAGGCAAAAGAGAAAGCGGAAGCAGAGTTGCAGAAGCTGAA | |
| | AATGATGTCTCCGATGTCGGCAGAAGCGACCGTAGTGCGTGGTTATATCGACTGGAT | |
| | GGTACAGGTACCGTGGAATGCGCGCAGCAAGGTCAAAAAAGACCTGCGTCAGGCGCA | |
| | GGAAATCCTTGATACCGACCATTATGGTCTGGAGCGCGTGAAAGATCGCATCCTTGA | |
| | GTACCTTGCGGTTCAAAGCCGTGTCAACAAAATCAAGGGACCAATCCTCTGCCTGGT | |
| | AGGGCCGCCGGGGGTAGGTAAAACCTCTCTTGGTCAGTCCATTGCCAAAGCCACCGG | |
| | GCGTAAATATGTCCGTATGGCGCTGGGCGGCGTGCGTGATGAAGCGGAAATCCGTGG | |
| | TCACCGCCGTACTTACATCGGTTCTATGCCGGGTAAACTGATCCAGAAAATGGCGAA | |
| | AGTGGGCGTGAAAAACCCGCTGTTCCTGCTCGATGAGATCGACAAAATGTCTTCTGA | |
| | CATGCGAGGCGATCCGGCCTCTGCACTGCTTGAAGTGCTGGATCCAGAGCAGAACGT | |
| | AGCGTTCAGCGACCACTACCTGGAAGTGGATTACGATCTCAGCGACGIGATGTTTGT | |
| | CGCGACGTCGAACTCCATGAACATTCCGGCACCGCTGCTGGATCGTATGGAAGTGAT | |
| | TCGCCTCTCCGGTTATACCGAAGATGAAAAACTGAACATCGCCAAACGTCACCTGCT | |
| | GCCGAAGCAGATTGAACGTAATGCACTGAAAAAAGGTGAGCTGACCGTCGACGATAG | |
| | CGCCATTATCGGCATTATTCGTTACTACACCCGTGAGGCGGGCGTGCGTGGTCTGGA | |
| | GCGTGAAATCTCCAAACTGTGCCGCAAAGCGGTTAAGCAGTTACTGCTCGATAAGTC | |
| | ATTAAAACATATCGAAATTAACGGCGATAACCTGCATGACTACCTCGGTGTTCAGCG | |
| | TTTCGACTATGGTCGCGCTGATAACGAAAACCGTGTCGGTCAGGTAACCGGTCTGGC | |
| | GTGGACGGAAGTGGGCGGTGACTTGCTGACCATTGAAACCGCATGTGTTCCGGGTAA | |
| | AGGCAAACTGACCTATACCGGTTCGCTCGGCGAAGTGATGCAGGAGTCTATTCAGGC | |
| | GGCGTTAACGGTGGTTCGTGCGCGTGCGGAAAAACTGGGGATCAACCCTGATTTTTA | |
| | CGAAAAACGTGACATCCACGTCCACGTACCGGAAGGTGCGACGCCGAAAGATGGTCC | |
| | GAGTGCCGGTATTGCTATGTGCACCGCGCTGGTTTCTTGCCTGACCGGTAACCCGGT | |
| | TCGTGCCGATGTGGCAATGACCGGTGAGATCACTCTGCGTGGTCAGGTACTGCCGAT | |
| | CGGTGGTTTGAAAGAAAAACTCCTGGCAGCGCATCGCGGCGGGATTAAAACAGTGCT | |
| | AATTCCGTTCGAAAATAAACGCGATCTGGAAGAGATTCCTGACAACGTAATTGCCGA | |
| | TCTGGACATTCATCCTGTGAAGCGCATTGAGGAAGTTCTGACTCTGGCGCTGCAAAA | |
| | TGAACCGTCCGGCATGCACCATTCCTTGCGGCGGCGGTGCTCAACGGCCTCAACCTA | |
| | CTACTGGGCTGCTTCCTAA | |
| 30 | MNPERSERIEIPVLPLRDVVVYPHMVIPLFVGREKSIRCLEAAMDHDKKIMLVAQKE | Lon amino acid |
| | ASTDEPGVNDLFTVGTVASILQMLKLPDGTVKVLVEGLQRARISALSDNGEHFSAKA | sequence |
| | EYLESPTIDEREQEVLVRTAISQFEGYIKLNKKIPPEVLTSLNSIDDPARLADTIAA | |
| | HMPLKLADKQSVLEMSDVNERLEYLMAMMESEIDLLQVEKRIRNRVKKQMEKSQREY | |
| | YLNEQMKAIQKELGEMDDAPDENEALKRKIDAAKMPKEAKEKAEAELQKLKMMSPMS | |
| | AEATVVRGYIDWMVQVPWNARSKVKKDLRQAQEILDTDHYGLERVKDRILEYLAVQS | |
| | RVNKIKGPILCLVGPPGVGKTSLGQSIAKATGRKYVRMALGGVRDEAEIRGHRRTYI | |
| | GSMPGKLIQKMAKVGVKNPLFLLDEIDKMSSDMRGDPASALLEVLDPEQNVAFSDHY | |
| | LEVDYDLSDVMFVATSNSMNIPAPLLDRMEVIRLSGYTEDEKLNIAKRHLLPKQIER | |
| | NALKKGELTVDDSAIIGIIRYYTREAGVRGLEREISKLCRKAVKQLLLDKSLKHIEI | |
| | NGDNLHDYLGVQRFDYGRADNENRVGQVTGLAWTEVGGDLLTIETACVPGKGKLTYT | |
| | GSLGEVMQESIQAALTVVRARAEKLGINPDFYEKRDIHVHVPEGATPKDGPSAGIAM | |
| | CTALVSCLTGNPVRADVAMTGEITLRGQVLPIGGLKEKLLAAHRGGIKTVLIPFENK | |
| | RDLEEIPDNVIADLDIHPVKRIEEVLTLALQNEPSGMQVVTAK | |
| 31 | CCGGACGCAGCATGAACGACGCATCATGAAACGCCTGGCGCGCCAACAGAGTTAACG | manA DNA |
| | CTTCGGGTGCGACTTTCAAAATCTCCTGCCCTTCAAATTCAGATACGCTAACGTGTT | coding |
| | CGCTGGTTAGCAGGTAATACTCAGTATCATCTTTTTTGAGTGGAAAAGGAGCCTGAT | sequence |
| | AATGAAAGGGTTTGTTTGACATTGTTCTCTCACTTACTGCCTGGTTTGGTTATGCTC | |
| | TGGGCGGGTGTTCCGTTGCCCTGTTAAAAGCGAGTAACAATATCCTACACACTTTTT | |
| | TAACAAAAACTGAGACTAGTACGACTTTTTGCGGCTCCAGGTTACTTCCCGTAGGAT | |
| | TCTTGCTTTAATAGTGGGATTAATTTCCACATTAAAACAGGGATTGATCATGCAAAA | |
| | ACTCATTAACTCAGTGCAAAACTATGCCTGGGGCAGCAAAACGGCGTTGACTGAACT | |
| | TTATGGTATGGAAAATCCGTCCAGCCAGCCGATGGCCGAGCTGTGGATGGGCGCACA | |
| | TCCGAAAAGCAGTTCACGAGTGCAGAATGCCGCCGGAGATATCGTTTCACTGCGTGA | |
| | TGTGATTGAGAGTGATAAATCGACTCTGCTCGGAGAGGCCGTTGCCAAACGCTTTGG | |
| | CGAACTGCCTTTCCTGTTCAAAGTATTATGCGCAGCACAGCCACTCTCCATTCAGGT | |
| | TCATCCAAACAAACACAATTCTGAAATCGGTTTTGCCAAAGAAAATGCCGCAGGTAT | |
| | CCCGATGGATGCCGCCGAGCGTAACTATAAAGATCCTAACCACAAGCCGGAGCTGGT | |
| | TTTTGCGCTGACGCCTTTCCTTGCGATGAACGCGTTTCGTGAATTTTCCGAGATTGT | |
| | CTCCCTACTCCAGCCGGTCGCAGGTGCACATCCGGCGATTGCTCACTTTTTACAACA | |
| | GCCTGATGCCGAACGTTTAAGCGAACTGTTCGCCAGCCTGTTGAATATGCAGGGTGA | |
| | AGAAAAATCCCGCGCGCTGGCGATTTTAAAATCGGCCCTCGATAGCCAGCAGGGTGA | |
| | ACCGTGGCAAACGATTCGTTTAATTTCTGAATTTTACCCGGAAGACAGCGGTCTGTT | |
| | CTCCCCGCTATTGCTGAATGTGGTGAAATTGAACCCTGGCGAAGCGATGTTCCTGTT | |
| | CGCTGAAACACCGCACGCTTACCTGCAAGGCGTGGCGCTGGAAGTGATGGCAAACTC | |
| | CGATAACGTGCTGCGTGCGGGTCTGACGCCTAAATACATTGATATTCCGGAACTGGT | |
| | TGCCAATGTGAAATTCGAAGCCAAACCGGCTAACCAGTTGTTGACCCAGCCGGTGAA | |
| | ACAAGGTGCAGAACTGGACTTCCCGATTCCAGTGGATGATTTTGCCTTCTCGCTGCA | |
| | TGACCTTAGTGATAAAGAAACCACCATTAGCCAGCAGAGTGCCGCCATTTTGTTCTG | |
| | CGTCGAAGGCGATGCAACGTTGTGGAAAGGTTCTCAGCAGTTACAGCTTAAACCGGG | |
| | TGAATCAGCGTTTATTGCCGCCAACGAATCACCGGTGACTGTCAAAGGCCACGGCCG | |
| | TTTAGCGCGTGTTTACAACAAGCTGTAAGAGCTTACTGAAAAAATTAACATCTCTTG | |
| | CTAAGCTT | |
| 32 | MQKLINSVQNYAWGSKTALTELYGMENPSSQPMAELWMGAHPKSSSRVQNAAGDIVS | ManA amino |
| | LRDVIESDKSTLLGEAVAKRFGELPFLFKVLCAAQPLSIQVHPNKHNSEIGFAKENA | acid |
| | AGIPMDAAERNYKDPNHKPELVFALTPFLAMNAFREFSEIVSLLQPVAGAHPAIAHF | sequence |
| | LQQPDAERLSELFASLLNMQGEEKSRALAILKSALDSQQGEPWQTIRLISEFYPEDS | |
| | GLFSPLLLNVVKLNPGEAMFLFAETPHAYLQGVALEVMANSDNVLRAGLTPKYIDIP | |
| | ELVANVKFEAKPANQLLTQPVKQGAELDFPIPVDDFAFSLHDLSDKETTISQQSAAI | |
| | LFCVEGDATLWKGSQQLQLKPGESAFIAANESPVTVKGHGRLARVYNKL | |
| 33 | TCGCGCTGCACTGGCGTAATGCTGACCGGATGGCTATCGCTAATGGTCTTACGCTCA | T7 RNA |
| | ACATTGATAAGCAACTTGACGCAATGTTAATGGGCTGATAGTCTTATCTTACAGGTC | polymerase |
| | ATCTGCGGGTGGCCTGAATAGGTACGATTTACTAACTGGAAGAGGCACTAAATGAAC | DNA coding |
| | ACGATTAACATCGCTAAGAACGACTTCTCTGACATCGAACTGGCTGCTATCCCGTTC | sequence |
| | AACACTCTGGCTGACCATTACGGTGAGCGTTTAGCTCGCGAACAGTTGGCCCTTGAG | |
| | CATGAGTCTTACGAGATGGGTGAAGCACGCTTCCGCAAGATGTTTGAGCGTCAACTT | |
| | AAAGCTGGTGAGGTTGCGGATAACGCTGCCGCCAAGCCTCTCATCACTACCCTACTC | |
| | CCTAAGATGATTGCACGCATCAACGACTGGTTTGAGGAAGTGAAAGCTAAGCGCGGC | |
| | AAGCGCCCGACAGCCTTCCAGTTCCTGCAAGAAATCAAGCCGGAAGCCGTAGCGTAC | |
| | ATCACCATTAAGACCACTCTGGCTTGCCTAACCAGTGCTGACAATACAACCGTTCAG | |
| | GCTGTAGCAAGCGCAATCGGTCGGGCCATTGAGGACGAGGCTCGCTTCGGTCGTATC | |
| | CGTGACCTTGAAGCTAAGCACTTCAAGAAAAACGTTGAGGAACAACTCAACAAGCGC | |
| | GTAGGGCACGTCTACAAGAAAGCATTTATGCAAGTIGTCGAGGCTGACATGCTCTCT | |
| | AAGGGTCTACTCGGTGGCGAGGCGTGGTCTTCGTGGCATAAGGAAGACTCTATTCAT | |
| | GTAGGAGTACGCTGCATCGAGATGCTCATTGAGTCAACCGGAATGGTTAGCTTACAC | |
| | CGCCAAAATGCTGGCGTAGTAGGTCAAGACTCTGAGACTATCGAACTCGCACCTGAA | |
| | TACGCTGAGGCTATCGCAACCCGTGCAGGTGCGCTGGCTGGCATCTCTCCGATGTTC | |
| | CAACCTTGCGTAGTTCCTCCTAAGCCGTGGACTGGCATTACTGGTGGTGGCTATTGG | |
| | GCTAACGGTCGTCGTCCTCTGGCGCTGGTGCGTACTCACAGTAAGAAAGCACTGATG | |
| | CGCTACGAAGACGTTTACATGCCTGAGGTGTACAAAGCGATTAACATTGCGCAAAAC | |
| | ACCGCATGGAAAATCAACAAGAAAGTCCTAGCGGTCGCCAACGTAATCACCAAGTGG | |
| | AAGCATTGTCCGGTCGAGGACATCCCTGCGATTGAGCGTGAAGAACTCCCGATGAAA | |
| | CCGGAAGACATCGACATGAATCCTGAGGCTCTCACCGCGTGGAAACGTGCTGCCGCT | |
| | GCTGTGTACCGCAAGGACAGGGCTCGCAAGTCTCGCCGTATCAGCCTTGAGTTCATG | |
| | CTTGAGCAAGCCAATAAGTTTGCTAACCATAAGGCCATCTGGTTCCCTTACAACATG | |
| | GACTGGCGCGGTCGTGTTTACGCCGTGTCAATGTTCAACCCGCAAGGTAACGATATG | |
| | ACCAAAGGACTGCTTACGCTGGCGAAAGGTAAACCAATCGGTAAGGAAGGTTACTAC | |
| | TGGCTGAAAATCCACGGTGCAAACTGTGCGGGTGTCGATAAGGTTCCGTTCCCTGAG | |
| | CGCATCAAGTTCATTGAGGAAAACCACGAGAACATCATGGCTTGCGCTAAGTCTCCA | |
| | CTGGAGAACACTTGGTGGGCTGAGCAAGATTCTCCGTTCTGCTTCCTTGCGTTCTGC | |
| | TTTGAGTACGCTGGGGTACAGCACCACGGCCTGAGCTATAACTGCTCCCTTCCGCTG | |
| | GCGTTTGACGGGTCTTGCTCTGGCATCCAGCACTTCTCCGCGATGCTCCGAGATGAG | |
| | GTAGGTGGTCGCGCGGTTAACTTGCTTCCTAGTGAGACCGTTCAGGACATCTACGGG | |
| | ATTGTTGCTAAGAAAGTCAACGAGATTCTACAAGCAGACGCAATCAATGGGACCGAT | |
| | AACGAAGTAGTTACCGTGACCGATGAGAACACTGGTGAAATCTCTGAGAAAGTCAAG | |
| | CTGGGCACTAAGGCACTGGCTGGTCAATGGCTGGCTCACGGTGTTACTCGCAGTGTG | |
| | ACTAAGCGTTCAGTCATGACGCTGGCTTACGGGTCCAAAGAGTTCGGCTTCCGTCAA | |
| | CAAGTGCTGGAAGATACCATTCAGCCAGCTATTGATTCCGGCAAGGGTCCGATGTTC | |
| | ACTCAGCCGAATCAGGCTGCTGGATACATGGCTAAGCTGATTTGGGAATCTGTGAGC | |
| | GTGACGGTGGTAGCTGCGGTTGAAGCAATGAACTGGCTTAAGTCTGCTGCTAAGCTG | |
| | CTGGCTGCTGAGGTCAAAGATAAGAAGACTGGAGAGATTCTTCGCAAGCGTTGCGCT | |
| | GTGCATTGGGTAACTCCTGATGGTTTCCCTGTGTGGCAGGAATACAAGAAGCCTATT | |
| | CAGACGCGCTTGAACCTGATGTTCCTCGGTCAGTTCCGCTTACAGCCTACCATTAAC | |
| | ACCAACAAAGATAGCGAGATTGATGCACACAAACAGGAGTCTGGTATCGCTCCTAAC | |
| | TTTGTACACAGCCAAGACGGTAGCCACCTTCGTAAGACTGTAGTGTGGGCACACGAG | |
| | AAGTACGGAATCGAATCTTTTGCACTGATTCACGACTCCTTCGGTACCATTCCGGCT | |
| | GACGCTGCGAACCTGTTCAAAGCAGTGCGCGAAACTATGGTTGACACATATGAGTCT | |
| | TGTGATGTACTGGCTGATTTCTACGACCAGTTCGCTGACCAGTTGCACGAGTCTCAA | |
| | TTGGACAAAATGCCAGCACTTCCGGCTAAAGGTAACTTGAACCTCCGTGACATCTTA | |
| | GAGTCGGACTTCGCGTTCGCGTAACGCCAAATCAATACGACTCACTATAGAGGGACA | |
| | AACTCAAGGTCATTCGCAAGAGTGGCC | |
| 34 | MNTINIAKNDFSDIELAAIPENTLADHYGERLAREQLALEHESYEMGEARFRKMFER | T7 RNA |
| | QLKAGEVADNAAAKPLITTLLPKMIARINDWFEEVKAKRGKRPTAFQFLQEIKPEAV | polymerase |
| | AYITIKTTLACLTSADNTTVQAVASAIGRAIEDEARFGRIRDLEAKHFKKNVEEQLN | amino acid |
| | KRVGHVYKKAFMQVVEADMLSKGLLGGEAWSSWHKEDSIHVGVRCIEMLIESTGMVS | sequence |
| | LHRQNAGVVGQDSETIELAPEYAEAIATRAGALAGISPMFQPCVVPPKPWTGITGGG | |
| | YWANGRRPLALVRTHSKKALMRYEDVYMPEVYKAINIAQNTAWKINKKVLAVANVIT | |
| | KWKHCPVEDIPAIEREELPMKPEDIDMNPEALTAWKRAAAAVYRKDKARKSRRISLE | |
| | FMLEQANKFANHKAIWFPYNMDWRGRVYAVSMFNPQGNDMTKGLLTLAKGKPIGKEG | |
| | YYWLKIHGANCAGVDKVPFPERIKFIEENHENIMACAKSPLENTWWAEQDSPFCFLA | |
| | FCFEYAGVQHHGLSYNCSLPLAFDGSCSGIQHFSAMLRDEVGGRAVNLLPSETVQDI | |
| | YGIVAKKVNEILQADAINGTDNEVVTVTDENTGEISEKVKLGTKALAGQWLAYGVTR | |
| | SVTKRSVMTLAYGSKEFGFRQQVLEDTIQPAIDSGKGLMFTQPNQAAGYMAKLIWES | |
| | VSVTVVAAVEAMNWLKSAAKLLAAEVKDKKTGEILRKRCAVHWVTPDGFPVWQEYKK | |
| | PIQTRLNLMFLGQFRLQPTINTNKDSEIDAHKQESGIAPNFVHSQDGSHLRKTVVWA | |
| | HEKYGIESFALIHDSFGTIPADAANLFKAVRETMVDTYESCDVLADFYDQFADQLHE | |
| | SQLDKMPALPAKGNLNLRDILESDFAFA | |
| 35 | GATGAGGTGCGCATTGTGGGGCAAACCGTTACACATAGACGCATACCTTGACAAGCG | T3 RNA |
| | TCTACAAGGCTGATAGAGTCTTTTCTTACAGGTCATCATGAGGIGGCCTGAATAGGA | polymerase |
| | ACGATTTATTCACAATGAGGTAAGCAATGAACATCATCGAAAACATCGAAAAGAATG | DNA coding |
| | ACTTCTCAGAAATCGAACTGGCTGCTATCCCGTTCAACACACTGGCTGACCACTACG | sequence |
| | GAAGCGCCTTGGCTAAAGAGCAGTTGGCTTTAGAACATGAGTCTTATGAGCTAGGCG | |
| | AGCGCCGCTTCCTCAAGATGCTTGAGCGTCAAGCGAAAGCTGGTGAGATTGCAGACA | |
| | ACGCAGCCGCTAAGCCGTTACTCGCTACGCTTCTCCCTAAGTTAACCACACGTATCG | |
| | TCGAGTGGCTCGAAGAGTACGCATCGAAGAAAGGCCGCAAGCCTAGCGCATACGCAC | |
| | CGCTCCAGTTACTCAAGCCGGAGGCCTCCGCGTTTATCACCCTGAAAGTTATCCTTG | |
| | CGTCACTAACCAGTACGAACATGACAACCATTCAGGCCGCTGCTGGTATGCTGGGGA | |
| | AAGCCATTGAGGACGAGGCACGATTTGGGCGCATCCGTGACCTAGAAGCGAAGCACT | |
| | TCAAGAAGCACGTTGAGGAACAGCTTAACAAGCGCCACGGGCAAGTCTACAAGAAAG | |
| | CATTTATGCAGGTGGTCGAGGCCGATATGATTGGTCGAGGTCTGCTTGGTGGCGAGG | |
| | CGTGGTCTAGCTGGGATAAAGAAACCACGATGCACGTAGGGATTCGCCTGATTGAAA | |
| | TGCTGATTGAATCCACGGGTCTGGTGGAATTACAGCGCCACAACGCAGGTAACGCAG | |
| | GCTCTGACCATGAGGCACTGCAACTGGCCCAAGAGTACGTGGACGTATTAGCGAAGC | |
| | GTGCAGGCGCTCTGGCGGGTATCTCTCCGATGTTCCAGCCGTGTGTCGTACCGCCGA | |
| | AACCTTGGGTAGCAATCACAGGGGGCGGCTATTGGGCTAACGGTCGCAGACCTTTGG | |
| | CACTCGTTCGCACTCACTCTAAGAAGGGCTTGATGCGCTACGAAGACGTTTACATGC | |
| | CAGAAGTCTACAAGGCTGTGAACCTCGCGCAAAACACCGCATGGAAAATCAACAAGA | |
| | AAGTTCTTGCTGTTGTCAATGAGATTGTTAACTGGAAGAATTGCCCGGTAGCAGACA | |
| | TTCCATCGCTGGAGCGCCAAGAGTTACCGCCTAAGCCTGACGACATTGACACCAACG | |
| | AGGCAGCGCTCAAGGAGTGGAAGAAAGCCGCTGCTGGTATCTATCGCTTGGACAAGG | |
| | CACGAGTGTCTCGCCGTATCAGCTTAGAGTTCATGCTGGAGCAGGCCAACAAGTTCG | |
| | CAAGTAAGAAAGCAATCTGGTTCCCTTACAACATGGACTGGCGCGGTCGTGTGTACG | |
| | CTGTGCCGATGTTCAACCCGCAAGGCAACGACATGACGAAAGGTCTGCTGACCCTTG | |
| | CTAAAGGCAAGCCAATCGGTGAGGAAGGTTTCTACTGGCTGAAAATCCACGGTGCGA | |
| | ACTGTGCGGGTGTTGATAAGGTTCCATTCCCGGAGCGCATCGCGTTCATTGAGAAGC | |
| | ACGTAGACGACATTCTGGCTTGCGCTAAAGACCCAATCAATAACACTTGGTGGGCTG | |
| | AGCAGGATTCACCGTTCTGTTTCCTCGCGTTTTGCTTCGAGTATGCAGGCGTTACGC | |
| | ACCACGGTCTGAGCTACAATTGCTCTCTGCCGCTGGCGTTCGACGGGTCTTGCTCTG | |
| | GTATCCAGCACTTCTCCGCGATGCTCCGCGATGAGGIAGGCGGTCGTGCGGTTAACC | |
| | TGCTGCCAAGCGAAACCGTGCAGGACATTTACGGCATCGTTGCACAGAAAGTAAACG | |
| | AGATTCTCAAACAGGATGCAATCAACGGCACGCCTAACGAGATGATTACCGTGACCG | |
| | ACAAGGACACCGGGGAAATCTCAGAGAAGCTCAAACTTGGAACCTCAACGCTGGCGC | |
| | AACAGTGGCTGGCATATGGTGTAACCCGTAGCGTAACTAAACGTTCGGTCATGACGC | |
| | TGGCTTACGGTTCCAAGGAGTTCGGCTTTCGTCAACAGGTATTGGATGACACCATTC | |
| | AGCCTGCAATTGACAGCGGTAAGGGCTTGATGTTCACCCAACCGAACCAAGCGGCTG | |
| | GCTATATGGCTAAGCTGATTTGGGATGCGGTAAGCGTGACCGTAGTTGCAGCGGTTG | |
| | AGGCGATGAACTGGCTCAAATCTGCCGCTAAGCTGCTGGCTGCTGAGGTCAAGGACA | |
| | AGAAGACCAAGGAGATTCTGCGCCACCGTTGCGCGGTTCACTGGACTACGCCGGACG | |
| | GCTTCCCGGTCTGGCAGGAATACCGCAAGCCACTCCAGAAGCGTCTCGATATGATTT | |
| | TCTTAGGGCAATTCCGTCTGCAACCGACGATTAATACCCTCAAGGATTCAGGCATTG | |
| | ACGCACACAAGCAGGAGTCTGGCATCGCTCCTAACTTTGTTCACTCACAGGACGGTA | |
| | GCCACCTCCGCATGACAGTCGTTTATGCTCACGAGAAGTATGGCATTGAGTCCTTTG | |
| | CGCTCATCCATGACAGCTTTGGGACTATCCCGGCAGACGCTGGTAAGCTCTTTAAGG | |
| | CTGTGCGTGAAACGATGGTTATCACCTATGAGAACAACGATGTGCTGGCAGACTTCT | |
| | ACTCTCAGTTTGCCGACCAGCTACACGAGACCCAACTGGACAAGATGCCTCCGCTTC | |
| | CGAAGAAAGGAAACCTGAACCTGCAAGACATTCTCAAGTCTGACTTTGCCTTTGCAT | |
| | AACAAGCACTTAGCATTAACCCTCACTAACGGGAGACTACTTAAGGTCTCCCACTTT | |
| | AAGACACTTTAGGTACTAAGAGATTAAATTTATGATTAACATTAAG | |
| 36 | MNIIENIEKNDFSEIELAAIPENTLADHYGSALAKEQLALEHESYELGERRFLKMLE | T3 RNA |
| | RQAKAGEIADNAAAKPLLATLLPKLTTRIVEWLEEYASKKGRKPSAYAPLQLLKPEA | polymerase |
| | SAFITLKVILASLTSTNMTTIQAAAGMLGKAIEDEARFGRIRDLEAKHFKKHVEEQL | amino acid |
| | NKRHGQVYKKAFMQVVEADMIGRGLLGGEAWSSWDKETTMHVGIRLIEMLIESTGLV | sequence |
| | ELQRHNAGNAGSDHEALQLAQEYVDVLAKRAGALAGISPMFQPCVVPPKPWVAITGG | |
| | GYWANGRRPLALVRTHSKKGLMRYEDVYMPEVYKAVNLAQNTAWKINKKVLAVVNEI | |
| | VNWKNCPVADIPSLERQELPPKPDDIDTNEAALKEWKKAAAGIYRLDKARVSRRISL | |
| | EFMLEQANKFASKKAIWFPYNMDWRGRVYAVPMFNPQGNDMTKGLLTLAKGKPIGEE | |
| | GFYWLKIHGANCAGVDKVPFPERIAFIEKHVDDILACAKDPINNTWWAEQDSPFCEL | |
| | AFCFEYAGVTHHGLSYNCSLPLAFDGSCSGIQHFSAMLRDEVGGRAVNLLPSETVQD | |
| | IYGIVAQKVNEILKQDAINGTPNEMITVTDKDTGEISEKLKLGTSTLAQQWLAYGVT | |
| | RSVTKRSVMTLAYGSKEFGFRQQVLDDTIQPAIDSGKGLMFTQPNQAAGYMAKLIWD | |
| | AVSVTVVAAVEAMNWLKSAAKLLAAEVKDKKTKEILRHRCAVHWTTPDGFPVWQEYR | |
| | KPLQKRLDMIFLGQFRLQPTINTLKDSGIDAHKQESGIAPNFVHSQDGSHLRMTVVY | |
| | AHEKYGIESFALIHDSFGTIPADAGKLFKAVRETMVITYENNDVLADFYSQFADQLH | |
| | ETQLDKMPPLPKKGNLNLQDILKSDFAFA | |
| 37 | GCGCTCAATTAAGTTTTCTAGTACCGCATGAGGATACAAGATGCAAGATTTACACGC | SP6 RNA |
| | TATCCAGCTTCAATTAGAAGAAGAGATGTTTAATGGTGGCATTCGTCGCTTCGAAGC | polymerase |
| | AGATCAACAACGCCAGATTGCAGCAGGTAGCGAGAGCGACACAGCATGGAACCGCCG | DNA coding |
| | CCTGTTGTCAGAACTTATTGCACCTATGGCTGAAGGCATTCAGGCTTATAAAGAAGA | sequence |
| | GTACGAAGGTAAGAAAGGTCGTGCACCTCGCGCATTGGCTTTCTTACAATGTGTAGA | |
| | AAATGAAGTTGCAGCATACATCACTATGAAAGTTGTTATGGATATGCTGAATACGGA | |
| | TGCTACCCTTCAGGCTATTGCAATGAGTGTAGCAGAACGCATTGAAGACCAAGTGCG | |
| | CTTTTCTAAGCTAGAAGGTCACGCCGCTAAATACTTTGAGAAGGTTAAGAAGTCACT | |
| | CAAGGCTAGCCGTACTAAGTCATATCGTCACGCTCATAACGTAGCTGTAGTTGCTGA | |
| | AAAATCAGTTGCAGAAAAGGACGCGGACTTTGACCGTTGGGAGGCGTGGCCAAAAGA | |
| | AACTCAATTGCAGATTGGTACTACCTTGCTTGAAATCTTAGAAGGTAGCGTTTTCTA | |
| | TAATGGTGAACCTGTATTTATGCGTGCTATGCGCACTTATGGCGGAAAGACTATTTA | |
| | CTACTTACAAACTTCTGAAAGTGTAGGCCAGTGGATTAGCGCATTCAAAGAGCACGT | |
| | AGCGCAATTAAGCCCAGCTTATGCCCCTTGCGTAATCCCTCCTCGTCCTTGGAGAAC | |
| | TCCATTTAATGGAGGGTTCCATACTGAGAAGGTAGCTAGCCGTATCCGTCTTGTAAA | |
| | AGGTAACCGTGAGCATGTACGCAAGTTGACTCAAAAGCAAATGCCAAAGGTTTATAA | |
| | GGCTATCAACGCATTACAAAATACACAATGGCAAATCAACAAGGATGTATTAGCAGT | |
| | TATTGAAGAAGTAATCCGCTTAGACCTTGGTTATGGTGTACCTTCCTTCAAGCCACT | |
| | GATTGACAAGGAGAACAAGCCAGCTAACCCGGTACCTGTTGAATTCCAACACCTGCG | |
| | CGGTCGTGAACTGAAAGAGATGCTATCACCTGAGCAGTGGCAACAATTCATTAACTG | |
| | GAAAGGCGAATGCGCGCGCCTATATACCGCAGAAACTAAGCGCGGTTCAAAGTCCGC | |
| | CGCCGTTGTTCGCATGGTAGGACAGGCCCGTAAATATAGCGCCTTTGAATCCATTTA | |
| | CTTCGTGTACGCAATGGATAGCCGCAGCCGTGTCTATGTGCAATCTAGCACGCTCTC | |
| | TCCGCAGTCTAACGACTTAGGTAAGGCATTACTCCGCTTTACCGAGGGACGCCCTGT | |
| | GAATGGCGTAGAAGCGCTTAAATGGTTCTGCATCAATGGTGCTAACCTTTGGGGATG | |
| | GGACAAGAAAACTTTTGATGTGCGCGTGTCTAACGTATTAGATGAGGAATTCCAAGA | |
| | TATGTGTCGAGACATCGCCGCAGACCCTCTCACATTCACCCAATGGGCTAAAGCTGA | |
| | TGCACCTTATGAATTCCTCGCTTGGTGCTTTGAGTATGCTCAATACCTTGATTTGGT | |
| | GGATGAAGGAAGGGCCGACGAATTCCGCACTCACCTACCAGTACATCAGGACGGGTC | |
| | TTGTTCAGGCATTCAGCACTATAGTGCTATGCTTCGCGACGAAGTAGGGGCCAAAGC | |
| | TGTTAACCTGAAACCCTCCGATGCACCGCAGGATATCTATGGGGCGGTGGCGCAAGT | |
| | GGTTATCAAGAAGAATGCGCTATATATGGATGCGGACGATGCAACCACGTTTACTTC | |
| | TGGTAGCGTCACGCTGTCCGGTACAGAACTGCGAGCAATGGCTAGCGCATGGGATAG | |
| | TATTGGTATTACCCGTAGCTTAACCAAAAAGCCCGTGATGACCTTGCCATATGGTTC | |
| | TACTCGCTTAACTTGCCGTGAATCTGTGATTGATTACATCGTAGACTTAGAGGAAAA | |
| | AGAGGCGCAGAAGGCAGTAGCAGAAGGGCGGACGGCAAACAAGGTACATCCTTTTGA | |
| | AGACGATCGTCAAGATTACTTGACTCCGGGCGCAGCTTACAACTACATGACGGCACT | |
| | AATCTGGCCTTCTATTTCTGAAGTAGTTAAGGCACCGATAGTAGCTATGAAGATGAT | |
| | ACGCCAGCTTGCACGCTTTGCAGCGAAACGTAATGAAGGCCTGATGTACACCCTGCC | |
| | TACTGGCTTCATCTTAGAACAGAAGATCATGGCAACCGAGATGCTACGCGTGCGTAC | |
| | CTGTCTGATGGGTGATATCAAGATGTCCCTTCAGGTTGAAACGGATATCGTAGATGA | |
| | AGCCGCTATGATGGGAGCAGCAGCACCTAATTTCGTACACGGTCATGACGCAAGTCA | |
| | CCTTATCCTTACCGTATGTGAATTGGTAGACAAGGGCGTAACTAGTATCGCTGTAAT | |
| | CCACGACTCTTTTGGTACTCATGCAGACAACACCCTCACTCTTAGAGTGGCACTTAA | |
| | AGGGCAGATGGTTGCAATGTATATTGATGGTAATGCGCTTCAGAAACTACTGGAGGA | |
| | GCATGAAGTGCGCTGGATGGTTGATACAGGTATCGAAGTACCTGAGCAAGGGGAGTT | |
| | CGACCTTAACGAAATCATGGATTCTGAATACGTATTTGCCTAATAGAACAATAAATA | |
| | TACAGGTCAGCCTTCGGGCTGGCCTTTTCTTTTAACTATTACCTGTAACATTTAATT | |
| | AACAAGTCCAACGTGTTGGACAC | |
| 38 | MQDLHAIQLQLEEEMFNGGIRRFEADQQRQIAAGSESDTAWNRRLLSELIAPMAEGI | SP6 RNA |
| | QAYKEEYEGKKGRAPRALAFLQCVENEVAAYITMKVVMDMLNTDATLQAIAMSVAER | polymerase |
| | IEDQVRFSKLEGHAAKYFEKVKKSLKASRTKSYRHAHNVAVVAEKSVAEKDADFDRW | amino acid |
| | EAWPKETQLQIGTTLLEILEGSVFYNGEPVFMRAMRTYGGKTIYYLQTSESVGQWIS | sequence |
| | AFKEHVAQLSPAYAPCVIPPRPWRTPFNGGFHTEKVASRIRLVKGNREHVRKLTQKQ | |
| | MPKVYKAINALQNTQWQINKDVLAVIEEVIRLDLGYGVPSFKPLIDKENKPANPVPV | |
| | EFQHLRGRELKEMLSPEQWQQFINWKGECARLYTAETKRGSKSAAVVRMVGQARKYS | |
| | AFESIYFVYAMDSRSRVYVQSSTLSPQSNDLGKALLRFTEGRPVNGVEALKWFCING | |
| | ANLWGWDKKTFDVRVSNVLDEEFQDMCRDIAADPLTFTQWAKADAPYEFLAWCFEYA | |
| | QYLDLVDEGRADEFRTHLPVHQDGSCSGIQHYSAMLRDEVGAKAVNLKPSDAPQDIY | |
| | GAVAQVVIKKNALYMDADDATTFTSGSVTLSGTELRAMASAWDSIGITRSLTKKPVM | |
| | TLPYGSTRLTCRESVIDYIVDLEEKEAQKAVAEGRTANKVHPFEDDRQDYLTPGAAY | |
| | NYMTALIWPSISEVVKAPIVAMKMIRQLARFAAKRNEGLMYTLPTGFILEQKIMATE | |
| | MLRVRTCLMGDIKMSLQVETDIVDEAAMMGAAAPNFVHGHDASHLILTVCELVDKGV | |
| | TSIAVIHDSFGTHADNTLTLRVALKGQMVAMYIDGNALQKLLEEHEVRWMVDTGIEV | |
| | PEQGEFDLNEIMDSEYVFA | |
| 39 | ACGAGGTGTATCTTAGGTGTCTCCGAACGGTGAGGCACCCATAGATAAACTTTATCC | K11 RNA |
| | ACAAAGAGGCACACAATGAACGCATTAAACATTGGACGTAATGACTTCTCCGAGATT | polymerase |
| | GAACTTGCTGCTATTCCGTACAACATCCTGAGCGAGCACTACGGGGACCAAGCGGCA | DNA coding |
| | CGTGAGCAGTTAGCACTGGAGCATGAGGCATACGAGCTTGGCAGACAACGTTTCCTG | sequence |
| | AAGATGTTAGAACGTCAGGTGAAAGCTGGTGAGTTCGCTGACAACGCGGCCGCTAAG | |
| | CCGCTGGTCTTAACGTTGCACCCACAGCTGACCAAGCGCATTGACGACTGGAAGGAG | |
| | GAGCAAGCAAACGCTCGCGGTAAGAAGCCTCGCGCATACTACCCGATTAAGCACGGC | |
| | GTCGCCTCAGAGTTAGCTGTTAGCATGGGCGCTGAGGTGCTAAAAGAGAAGCGCGGA | |
| | GTGTCCAGTGAGGCAATCGCACTGCTGACCATTAAGGTCGTCTTGGGGAACGCTCAC | |
| | AGACCGCTCAAAGGCCACAATCCAGCAGTATCCTCTCAGTTAGGCAAGGCTCTTGAG | |
| | GATGAGGCCCGCTTCGGTCGTATCCGTGAGCAGGAAGCCGCATACTTCAAGAAGAAC | |
| | GTAGCGGACCAGCTGGACAAGCGAGTAGGCCACGTGTACAAGAAGGCTTTCATGCAG | |
| | GTAGTCGAGGCCGATATGATATCCAAAGGGATGCTGGGCGGCGACAACTGGGCGAGC | |
| | TGGAAAACTGACGAGCAGATGCACGTAGGGACCAAGCTGCTGGAGCTACTCATTGAG | |
| | GGAACTGGTCTGGTGGAAATGACCAAGAACAAGATGGCCGATGGCTCCGATGATGTA | |
| | ACCAGTATGCAGATGGTCCAGCTGGCTCCGGCCTTTGTGGAACTCCTGAGCAAACGG | |
| | GCAGGCGCACTCGCGGGTATCAGCCCGATGCACCAGCCGTGCGTAGTCCCTCCGAAA | |
| | CCTTGGGTGGAGACCGTAGGCGGTGGCTACTGGTCAGTCGGTCGCCGTCCGCTGGCA | |
| | CTGGTGCGTACCCACTCCAAGAAGGCGCTGCGCCGCTACGCTGACGTGCACATGCCA | |
| | GAGGTATACAAGGCGGTAAACCTCGCGCAAAACACGCCGTGGAAGGTGAACAAGAAG | |
| | GTGCTGGCGGTAGTCAACGAGATTGTCAACTGGAAGCACTGCCCGGTAGGTGACGTC | |
| | CCAGCGATTGAACGCGAAGAGTTACCGCCGCGCCCGGACGATATTGACACCAACGAG | |
| | GTGGCACGTAAGGCATGGCGCAAGGAGGCCGCAGCGGTCTACCGTAAGGACAAGGCC | |
| | CGCCAGTCTCGCCGTTGTCGATGTGAGTTCATGGTCGCACAGGCTAACAAGITCGCT | |
| | AACCACAAGGCCATTTGGTTCCCGTACAACATGGACTGGCGCGGGCGTGTGTACGCT | |
| | GTGAGCATGTTCAACCCACAGGGTAACGATATGACCAAGGGGAGTCTGACGCTGGCC | |
| | AAGGGTAAGCCAATTGGTCTCGACGGGTTCTACTGGCTGAAGATTCACGGCGCAAAC | |
| | TGTGCAGGTGTCGACAAGGTTCCCTTCCCTGAGCGCATCAAGTTCATCGAAGAGAAC | |
| | GAGGGCAACATTCTGGCGAGCGCAGCGGACCCGCTGAATAACACTTGGTGGACCCAG | |
| | CAAGATTCGCCGTTCTGTTTCTTAGCGTTCTGCTTCGAGTACGCAGGTGTTAAGCAT | |
| | CACGGCCTGAATTACAACTGCTCGCTGCCGCTGGCGTTCGATGGGTCCTGCTCTGGG | |
| | ATTCAGCACTTCAGCGCGATGCTCCGAGATTCCATCGGTGGTCGTGCGGTTAACCTG | |
| | CTGCCTTCTGATACCGTGCAGGATATCTACAAGATTGTGGCCGACAAGGTGAACGAA | |
| | GTGCTCCACCAGCACGCCGTCAACGGGTCTCAGACCGTGGTCGAGCAGATTGCTGAC | |
| | AAAGAGACTGGCGAGTTTCACGAGAAGGTGACTCTGGGCGAGTCCGTACTGGCTGCG | |
| | CAGTGGTTGCAATATGGTGTGACCCGCAAGGTGACTAAGCGTTCGGTCATGACGCTG | |
| | GCATACGGTTCCAAAGAGTCGTTGGTCCGCCAGCAGGTTCTTGAGGACACCATTCAG | |
| | CCTGCTATTGACAACGGCGAGGGCCTGATGTTTACGCACCCTAACCAAGCAGCTGGC | |
| | TACATGGCTAAGCTGATTTGGGACGCTGTGACCGTGACCGTAGTGGCCGCTGTCGAG | |
| | GCAATGAACTGGCTGAAGTCTGCCGCTAAGCTGCTGGCTGCTGAAGTCAAGGACAAG | |
| | AAGACCAAAGAGGTGCTGCGTAAGCGCTGCGCAATCCACTGGGTAACACCCGATGGC | |
| | TTCCCGGTGTGGCAGGAGTACCGCAAGCAGAACCAAGCGCGCCTGAAGCTGGTCTTC | |
| | CTCGGGCAGGCCAACGTCAAGATGACGTATAACACTGGGAAGGACTCCGAGATTGAT | |
| | GCCCACAAGCAGGAATCCGGCATCGCTCCTAACTTTGTTCACTCACAGGATGGCAGT | |
| | CACCTGCGCATGACTGTAGTACACGCCAACGAGGTCTACGGGATTGACTCCTTCGCA | |
| | CTCATTCACGACTCCTCTGGGACCATTCCGGCTGACGCTGGGAATCTCTTTAAGGCA | |
| | GTCCGCGAGACGATGGTCAAGACCTACGAGGACAACGATGTAATTGCAGACTTCTAC | |
| | GACCAGTTTGCCGACCAGCTGCACGAGTCTCAACTGGACAAGATGCCTGCGGTTCCG | |
| | GCCAAAGGTGACCTGAATCTGCGCGATATCTTGGAGTCTGACTTCGCGTTTGCGTAA | |
| | GGTCTCAGGCAATTAGGGCACACTATAGGGAACCTTCGAATGACCGAGGG | |
| 40 | MNALNIGRNDFSEIELAAIPYNILSEHYGDQAAREQLALEHEAYELGRQRFLKMLER | K11 RNA |
| | QVKAGEFADNAAAKPLVLTLHPQLTKRIDDWKEEQANARGKKPRAYYPIKHGVASEL | polymerase |
| | AVSMGAEVLKEKRGVSSEAIALLTIKVVLGNAHRPLKGHNPAVSSQLGKALEDEARF | amino acid |
| | GRIREQEAAYFKKNVADQLDKRVGHVYKKAFMQVVEADMISKGMLGGDNWASWKTDE | sequence |
| | QMHVGTKLLELLIEGTGLVEMTKNKMADGSDDVTSMQMVQLAPAFVELLSKRAGALA | |
| | GISPMHQPCVVPPKPWVETVGGGYWSVGRRPLALVRTHSKKALRRYADVHMPEVYKA | |
| | VNLAQNTPWKVNKKVLAVVNEIVNWKHCPVGDVPAIEREELPPRPDDIDTNEVARKA | |
| | WRKEAAAVYRKDKARQSRRCRCEFMVAQANKFANHKAIWFPYNMDWRGRVYAVSMEN | |
| | PQGNDMTKGSLTLAKGKPIGLDGFYWLKIHGANCAGVDKVPFPERIKFIEENEGNIL | |
| | ASAADPLNNTWWTQQDSPFCFLAFCFEYAGVKHHGLNYNCSLPLAFDGSCSGIQHES | |
| | AMLRDSIGGRAVNLLPSDTVQDIYKIVADKVNEVLHQHAVNGSQTVVEQIADKETGE | |
| | FHEKVTLGESVLAAQWLQYGVTRKVTKRSVMTLAYGSKESLVRQQVLEDTIQPAIDN | |
| | GEGLMFTHPNQAAGYMAKLIWDAVTVTVVAAVEAMNWLKSAAKLLAAEVKDKKTKEV | |
| | LRKRCAIHWVTPDGFPVWQEYRKQNQARLKLVFLGQANVKMTYNTGKDSEIDAHKQE | |
| | SGIAPNFVHSQDGSHLRMTVVHANEVYGIDSFALIHDSSGTIPADAGNLFKAVRETM | |
| | VKTYEDNDVIADFYDQFADQLHESQLDKMPAVPAKGDLNLRDILESDFAFA | |
| 41 | MNTINIAKNDFSDIELAAIPENTLADHYGERLAREQLALEHESYEMAEARFRKMFER | T7 RNA |
| | QLKAGEVADNAAAKPLITTLLPKMIARINDWFEEVKAKRGKRPTAFQFLQEIKPEAV | polymerase |
| | AYITIKTTLACLTSADNTTVQAVASAIGRAIEDEARFGRIRDLEAKHFKKNVEEQLN | variant 1 |
| | KRVGHVYKKAFMQVVEADMLSKGLLGGEAWSSWHKEDSIHVGVRCIEMLIESTGMVS | amino acid |
| | LHRQNAGVVGQDSETIELAPEYAEAIATRAGALAGISPMFQPCVVPPKPWTGITGGG | sequence |
| | YWANGRRPLALVRTHSKKALMRYEDVYMPEVYKAINIAQNTAWKINKKVLAVANVIT | |
| | KWKHCPVEDIPAIEREELPMKPEDIDMNPEALTAWKRAAAAVYRKDKARKSRRISLE | |
| | FMLEQANKFANHKAIWFPYNMDWRGRVYAVSMFNPQGNDMTKGLLTLAKGKPIGKEG | |
| | YYWLKIHGANCAGVDKVPFPERIKFIEENHENIMACAKSPLENTWWAEQDSPFCFLA | |
| | FCFEYAGVQHHGLSYNCSLPLAFDGSCSGIQHFSAMLRDEVGGRAVNLLPSETVQDI | |
| | YGIVAKKVNEILQADAINGTDNEVVTVTDENTGEISEKVKLGTKALAGQWLAYGVTR | |
| | SVTKRSVMTLAYGSKEFGFRQQVLEDTIQPAIDSGKGLMFTQPNQAAGYMAKLIWES | |
| | VSVTVVAAVEAMNWLKSAAKLLAAEVKDKKTGEILRKRCAVHWVTPDGFPVWQEYKK | |
| | PIQTRLNLMFLGQFRLQPTINTNKDSEIDAHKQESGIAPNFVHSQDGSHLRKTVVWA | |
| | HEKYGIESFALIHDSFGTIPADAANLFKAVRETMVDTYESCDVLADFYDQFADQLHE | |
| | SQLDKMPALPAKGNLNLRDILESDFAFAG | |
| 42 | MNTINIAKNDFSDIELAAIPENTLADHYGERLAREQLALEHESYEMAEARFRKMFER | T7 RNA |
| | QLKAGEVADNAAAKPLITTLLPKMIARINDWFEEVKAKRGKRPTAFQFLQEIKPEAV | polymerase |
| | AYITIKTTLACLTSADNTTVQAVASAIGRAIEDEARFGRIRDLEAKHFKKNVEEQLN | variant 2 |
| | KRVGHVYKKAFMQVVEADMLSKGLLGGEAWSSWHKEDSIHVGVRCIEMLIESTGMVS | amino acid |
| | LHRQNAGVVGQDSETIELAPEYAEAIATRAGALAGISPMFQPCVVPPKPWTGITGGG | sequence |
| | YWANGRRPLALVRTHSKKALMRYEDVYMPEVYKAINIAQNTAWKINKKVLAVANVIT | |
| | KWKHCPVWVIPAIEREELPMKPEDIDMNPEALTAWKRAAAAVYRKDKARKSRRISLE | |
| | FMLEQANKFANHKAIWFPYNMDWRGRVYAVSMFNPQGNDMTKGLLTLAKGKPIGKEG | |
| | YYWLKIHGANCAGVDKVPFPERIKFIEENHENIMACAKSPLENTWWAEQDSPFCFLA | |
| | FCFEYAGVQHHGLSYNCSLPLAFDGSCSGIQHFSAMLRDEVGGRAVNLLPSETVQDI | |
| | YGIVAKKVNEILQADAINGTDNEVVTVTDENTGEISEKVKLGTKALAGQWLAYGVTR | |
| | SVTKRSVMTLAYGSKEFGFRQQVLEDTIQPAIDSGKGLMFTQPNQAAGYMAKLIWES | |
| | VSVTVVAAVEAMNWLKSAAKLLAAEVKDKKTGEILRKRCAVHWVTPDGFPVWQEYKK | |
| | PIQTRLNLMFLGQFRLQPTINTNKDSEIDAHKQESGIAPNFVHSQDGSHLRKTVVWA | |
| | HEKYGIESFALIHDSFGTIPADAANLFKAVRETMVDTYESCDVLADFYDQFADQLHE | |
| | SQLDKMPALPAKGNLNLRDILESDFAFAG | |
| 43 | MNTINIAKNDFSDIELAAIPFNTLADHYGERLAREQLALEHESYEMGEARFRKMFER | T7 RNA |
| | QLKAGEVADNAAAKPLITTLLPKMIARINDWFEEVKAKRGKRPTAFQFLQEIKPEAV | polymerase |
| | AYITIKTTLACLTSADNTTVQAVASAIGRAIEDEARFGRIRDLEAKHFKKNVEEQLN | variant 3 |
| | KRVGHVYKKAFMQVVEADMLSKGLLGGEAWSSWHKEDSIHVGVRCIEMLIESTGMVS | amino acid |
| | LHRQNAGVVGQDSETIELAPEYAEAIATRAGALAGISPMFQPCVVPPKPWTGITGGG | sequence |
| | YWANGRRPLALVRTHSKKALMRYEDVYMPEVYKAINIAQNTAWKINKKVLAVANVIT | |
| | KWKHCPVWVIPAIEREELPMKPEDIDMNPEALTAWKRAAAAVYRKDKARKSRRISLE | |
| | FMLEQANKFANHKAIWFPYNMDWRGRVYAVSMFNPQGNDMTKGLLTLAKGKPIGKEG | |
| | YYWLKIHGANCAGVDKVPFPERIKFIEENHENIMACAKSPLENTWWAEQDSPFCFLA | |
| | FCFEYAGVQHHGLSYNCSLPLAFDGSCSGIQHFSAMLRDEVGGRAVNLLPSETVQDI | |
| | YGIVAKKVNEILQADAINGTDNEVVTVIDENTGEISEKVKLGTKALAGQWLAYGVTR | |
| | SVTKRSVMTLAYGSKEFGFRQQVLEDTIQPAIDSGKGLMFTQPNQAAGYMAKLIWES | |
| | VSVTVVAAVEAMNWLKSAAKLLAAEVKDKKTGEILRKRCAVHWVTPDGFPVWQEYKK | |
| | PIQTRLNLMFLGQFRLQPTINTNKDSEIDAHKQESGIAPNFVHSQDGSHLRKTVVWA | |
| | HEKYGIESFALIHDSFGTIPADAANLFKAVRETMVDTYESCDVLADFYDQFADQLHE | |
| | SQLDKMPALPAKGNLNLRDILESDFAFA | |
| 44 | MYRYLSIAAVVLSAAFSGPALAEGINSFSQAKAAAVKVHADAPGTFYCGCKINWQGK | EndA amino |
| | KGVVDLQSCGYQVRKNENRASRVEWEHVVPAWQFGHQRQCWQDGGRKNCAKDPVYRK | acid |
| | MESDMHNLQPSVGEVNGDRGNFMYSQWNGGEGQYGQCAMKVDFKEKAAEPPARARGA | sequence |
| | IARTYFYMRDQYNLTLSRQQTQLFNAWNKMYPVTDWECERDERIAKVQGNHNPYVQR | |
| | ACQARKS | |
| 45 | MAIDENKQKALAAALGQIEKQFGKGSIMRLGEDRSMDVETISTGSLSLDIALGAGGL | RecA amino |
| | PMGRIVEIYGPESSGKTTLTLQVIAAAQREGKICAFIDAEHALDPIYARKLGVDIDN | acid |
| | LLCSQPDTGEQALEICDALARSGAVDVIVVDSVAALTPKAEIEGEIGDSHMGLAARM | sequence |
| | MSQAMRKLAGNLKQSNTLLIFINQIRMKIGVMFGNPETTTGGNALKFYASVRLDIRR | |
| | IGAVKEGENVVGSETRVKVVKNKIAAPFKQAEFQILYGEGINFYGELVDLGVKEKLI | |
| | EKAGAWYSYKGEKIGQGKANATAWLKDNPETAKEIEKKVRELLLSNPNSTPDESVDD | |
| | SEGVAETNEDF | |
| 46 | MTVPTYDKFIEPVLRYLATKPEGAAARDVHEAAADALGLDDSQRAKVITSGQLVYKN | Mrr amino |
| | RAGWAHDRLKRAGLSQSLSRGKWCLTPAGFDWVASHPQPMTEQETNHLAFAFVNVKL | acid |
| | KSRPDAVDLDPKADSPDHEELAKSSPDDRLDQALKELRDAVADEVLENLLQVSPSRF | sequence |
| | EVIVLDVLHRLGYGGHRDDLQRVGGTGDGGIDGVISLDKLGLEKVYVQAKRWQNTVG | |
| | RPELQAFYGALAGQKAKRGVFITTSGFTSQARDFAQSVEGMVLVDGERLVHLMIENE | |
| | VGVSSRLLKVPKLDMDYFE | |
| 47 | agagttgggcaacggatgtgctggtggaggtgatcgcctcctgatgatgagccgctc | Mrr-hsdRMS- |
| | ccgatgtggtgtcgggagcggtattttctataaaacttaccgctcactcaaaatagt | symRE-mcrBC |
| | ccatatccagtttcggcaccttcaacaaacgtgaagaaacccctacttcgttttcga | locus in |
| | tcattaagtgcaccaggcgttccccatcaaccaacaccataccctcgacggattggg | wildtype |
| | caaagtcacgcgcctgagaagtaaatccagaagtggtaataaacaccccacgtttcg | E coli MG1655 |
| | ctttttgcccagccagtgcgccgtaaaatgcctgtaattctggcctgcctacagtat | (sequence |
| | tctgccaacgttttgcctgaacataaactttctccaggccaagtttatcaagcgata | upstream |
| | tcacaccatcgatgccaccatctccagtaccgccaacacgctgcaaatcatcacggt | (single |
| | ggccgccataccccaggcgatgcaaaacatccagaacaatgacttcaaagcgcgaag | underline) |
| | gagaaacctgcaataagttttccagaacctcatcagccaccgcatcacgaagctctt | and |
| | ttagcgcctgatctaaccgatcgtccgggctgctctttgcaagttcttcatgatcgg | downstream |
| | gagagtcggctttcggatctaaatcgacggcatccggccgtgacttaagtttgacat | (italics) of |
| | tcacaaaagcgaaggccagatggttcgtctcctgctccgtcattggctggggatgag | region to be |
| | acgcaacccagtcaaaacccgcaggagtcaggcaccatttgccacgcgacaaacttt | deleted) |
| | gcgacaacccggcacgttttaaacggtcatgcgcccagcctgcacgatttttataaa | |
| | caagttgtccgctggtaatgactttcgctcgctggctgtcatccagtcctaatgcat | |
| | ccgcggcagcctcatgaacatcacgcgcggctgcaccttccggttttgttgccagat | |
| | aacgcagaacaggttcaataaatttgtcataggtaggaaccgtcatagtacatcctt | |
| | gcagaatcaggtagatgtttttcggctactatagcactacaaaaatagacgaacacg | |
| | ttagaaatgagtcagttgctgtgaccgtggtcattgcccggaaaggtacagaaagct | |
| | aagatgagatgttatgggccttaaatatttggacaggcccgcacagcaatggattaa | |
| | taacaatgatgaataaatccaattttgaattcctgaagggcgtcaacgacttcactt | |
| | atgccatcgcctgtgcggcggaaaataactacccggatgatcccaacacgacgctga | |
| | ttaaaatgcgtatgtttggcgaagccacagcgaaacatcttggtctgttactcaaca | |
| | tccccccttgtgagaatcaacacgatctcctgcgtgaactcggcaaaatcgcctttg | |
| | ttgatgacaacatcctctctgtatttcacaaattacgccgcattggtaaccaggcgg | |
| | tgcacgaatatcataacgatctcaacgatgcccagatgtgcctgcgactcgggttcc | |
| | gcctggctgtctggtactaccgtctggtcactaaagattatgacttcccggtgccgg | |
| | tgtttgtgttgccggaacgtggtgaaaacctctatcaccaggaagtgctgacgctaa | |
| | aacaacagcttgaacagcaggtgcgagaaaaagcgcagactcaggcagaagtcgaag | |
| | cgcaacagcagaagctggttgccctgaacggctatatcgccattctggaaggcaaac | |
| | agcaggaaaccgaagcgcaaacccaggctcgccttgcggcactggaagcacagctcg | |
| | ccgagaagaacgcggaactggcaaaacagaccgaacaggaacgtaaggcttaccaca | |
| | aagaaattaccgatcaggccatcaagcgcacactcaaccttagcgaagaagagagtc | |
| | gcttcctgattgatgcgcaactgcgtaaagcaggctggcaggccgacagcaaaaccc | |
| | tgcgcttctccaaaggcgcacgtccggaacccggcgtcaataaagccattgccgaat | |
| | ggccgaccggaaaagatgaaacgggtaatcagggctttgcggattatgtgctgtttg | |
| | tcggcctcaaacccatcgcggtggtagaggcgaaacgtaacaatatcgacgttcccg | |
| | ccaggctcaatgagtcgtatcgctacagtaaatgtttcgataatggcttcctgcggg | |
| | aaaccttgcttgagcactactcaccggatgaagtgcatgaagcagtgccagagtatg | |
| | aaaccagctggcaggacaccagcggcaaacaacggtttaaaatccccttctgctact | |
| | cgaccaacgggcgcgaataccgcgcaacaatgaagaccaaaagcggcatctggtatc | |
| | gcgacgtgcgtgatacccgcaatatgtcgaaagccttacccgagtggcaccgcccgg | |
| | aagagctgctggaaatgctcggcagcgaaccgcaaaaacagaatcagtggtttgccg | |
| | ataaccctggcatgagcgagctgggcctgcgttattatcaggaagatgccgtccgcg | |
| | cggttgaaaaggcaatcgtcaaggggcaacaagagatcctgctggcgatggcgaccg | |
| | gtaccggtaaaacccgtacggcaatcgccatgatgttccgcctgatccagtcccagc | |
| | gttttaaacgcattctcttccttgtcgaccgccgttctcttggcgaacaggcgctgg | |
| | gcgcgtttgaagatacgcgtattaacggcgacaccttcaacagcattttcgacatta | |
| | aagggctgacggataaattcccggaagacagcaccaaaattcacgttgccaccgtac | |
| | agtcgctggtgaaacgcaccctgcaatcagatgaaccgatgccggtggcccgttacg | |
| | actgtatcgtcgttgacgaagcgcatcgcggctatattctcgataaagagcagaccg | |
| | aaggcgaactgcagttccgcagccagctggattacgtctctgcctaccgtcgcattc | |
| | tcgatcacttcgatgcggtaaaaatcgctctcaccgccaccccggcgctacatactg | |
| | tgcagattttcggcgagccggtttaccgttatacctaccgtaccgcggttatcgacg | |
| | gttttctgatcgaccaggatccgcctattcagatcatcacccgcaacgcgcaggagg | |
| | gggtttatctctccaaaggcgagcaggtagagcgcatcagcccgcagggagaagtga | |
| | tcaatgacaccctggaagacgatcaggattttgaagtcgccgactttaaccgtggcc | |
| | tggtgatcccggcgtttaaccgcgccgtctgtaacgaactcaccaattatcttgacc | |
| | cgaccggatcgcaaaaaacgctggtcttctgcgtcaccaatgcccatgccgatatgg | |
| | tggtggaagagctgcgtgccgcgttcaagaaaaagtatccgcaactggagcacgacg | |
| | cgatcatcaagatcaccggtgatgccgataaagacgcgcgcaaagtgcagaccatga | |
| | tcacccgcttcaataaagagcggctgcccaatatcgtggtaaccgtcgacctgctga | |
| | cgaccggcgtcgatattccgtcgatctgtaatatcgtgttcctgcgtaaagtacgca | |
| | gccgcattctgtacgaacagatgaaaggccgcgccacgcgcttatgcccggaggtga | |
| | ataaaaccagctttaagatttttgactgtgtcgatatctacagcacgctggagagcg | |
| | tcgacaccatgcgtccggtggtggtgcgcccgaaggtggaactgcaaacgctggtca | |
| | atgaaattaccgattcagaaacctataaaatcaccgaagcggatggccgcagttttg | |
| | ccgagcacagccatgaacaactggtggcgaagctccagcgtatcatcggtctggcca | |
| | cgtttaaccgtgaccgcagcgaaacgatagataaacaggtgcgtcgtctggatgagc | |
| | tatgccaggacgcgggggcgtgaactttaacggcttcgcctcgcgcctgcgggaaa | |
| | aagggccgcactggagcgccgaagtctttaacaaactgcctggctttatcgcccgtc | |
| | tggaaaagctgaaaacggacatcaacaacctgaatgatgcgccgatcttcctcgata | |
| | tcgacgatgaagtggtgagtgtaaaatcgctgtacggtgattacgacacgccgcagg | |
| | atttcctcgaagcctttgactcgctggtgcaacgttccccgaacgcgcaaccggcat | |
| | tgcaggcagttattaatcgcccgcgcgatctcacccgtaaagggctggtcgagctac | |
| | aggagtggtttgaccgccagcactttgaggaatcttccctgcgcaaagcatggaaag | |
| | agacgcgcaatgaagatatcgccgcccggctgattggtcatattcgccgcgctgcgg | |
| | tgggcgatgcgctgaaaccgtttgaggaacgtgtcgatcacgcgctgacgcgcatta | |
| | agggcgaaaacgactggagcagcgagcaattaagctggctcgatcgtttagcgcagg | |
| | cgctgaaagagaaagtggtgctcgacgacgatgtcttcaaaaccggcaacttccacc | |
| | gtcgcggcgggaaggcgatgctgcaaagaacctttgacgataatctcgataccctgc | |
| | tgggcaaattcagcgattatatctgggacgagctggcctgacacgtatacacttcat | |
| | ccttcaggctgcctctgcgttggctgcgctcgttcaccccggtcacgtacttctgta | |
| | cgctcccggggattcactcacttgccgccttgatgcaacctgaatgattttgtgtat | |
| | attaccctcggcaatttcttcttctgcggctcgatgaatttgggccgctgcttaatt | |
| | tacggaactcacaatgaacaataacgatctggtcgcgaagctgtggaagctgtgcga | |
| | caacctgcgcgatggcggcgtttcctatcaaaactacgtcaatgaactcgcctcgct | |
| | gctgtttttgaaaatgtgtaaagagaccggtcaggaagcggaatacctgccggaagg | |
| | ttaccgctgggatgacctgaaatcccgcatcggccaggagcagttgcagttctaccg | |
| | aaaaatgctcgtgcatttaggcgaagatgacaaaaagctggtacaggcagtttttca | |
| | taatgttagtaccaccatcaccgagccgaaacaaataaccgcactggtcagcaatat | |
| | ggattcgctggactggtacaacggcgcgcacggtaagtcgcgcgatgacttcggcga | |
| | tatgtacgaagggctgttgcagaagaacgcgaatgaaaccaagtctggtgcaggcca | |
| | gtacttcaccccgcgtccgctgattaaaaccattattcatctgctgaaaccgcagcc | |
| | gcgtgaagtggtgcaggacccggcggcaggtacggcgggctttttgattgaagccga | |
| | ccgctatgttaagtcgcaaaccaatgatctggacgaccttgatggcgacacgcagga | |
| | tttccagatccaccgcgcgtttatcggcctcgaactggtgcccggcacccgtcgtct | |
| | ggcactgatgaactgcctgctgcacgatattgaaggcaacctcgaccacggcggcgc | |
| | aatccgtctgggcaacactctgggtagcgacggtgaaaacctgccgaaggcgcatat | |
| | tgtcgccactaacccgccgtttggcagcgccgcaggcaccaacattacccgcacctt | |
| | tgttcacccgaccagcaacaaacagttgtgctttatgcagcatattatcgaaacgct | |
| | gcatcccggcggtcgtgcggcggtggtggtgccggataacgtgctgtttgaaggcgg | |
| | caaaggcaccgacattcgtcgtgacctgatggataagtgtcatctgcacaccattct | |
| | gcgtctgccgaccggtattttttacgctcagggcgtgaagaccaacgtgctgttctt | |
| | taccaaagggacggtggcgaacccgaatcaggataagaactgtaccgatgatgtgtg | |
| | ggtgtatgacctgcgtaccaatatgccgagtttcggcaagcgcacaccgtttaccga | |
| | cgagcatttgcagccgtttgagcgcgtgtatggcgaagacccgcacggtttaagccc | |
| | gcgcactgaaggtgaatggagttttaacgccgaagagacggaagttgccgacagcga | |
| | agagaacaaaaacaccgaccagcatcttgctaccagccgctggcgcaagttcagccg | |
| | tgagtggatccgcaccgcaaaatccgattcgctggatatctcctggctgaaagataa | |
| | agacagtattgatgccgacagcctgccggagccggatgtattagcggcagaagcgat | |
| | gggcgaactggtacaggcgctgtctgaactggatgcgctgatgcgtgaactgggggc | |
| | gagcgatgaggccgatttgcagcgtcagttgctggaagaagcgtttggtggggtgaa | |
| | ggaatgagtgcggggaaattgccggaggggtgggttatcgccccagtatctacggtc | |
| | acaactctaatccgaggagtaacgtataaaaaagagcaggcaataaattatctaaaa | |
| | gatgattatttgcctcttatccgtgcgaacaatattcagaatggcaagtttgatact | |
| | acggacttggtttttgttcctaaaaatcttgttaaagaaagtcaaaaaatatctcct | |
| | gaagatattgttattgcaatgtcatcagggagcaaatccgtagttggtaaatccgca | |
| | catcagcatctaccatttgaatgtagtttcggcgcattttgcggtgtattacgtcct | |
| | gaaaaacttatattttctggttttattgctcatttcacaaaatcttctctttatcga | |
| | aacaaaatttcatcactttctgctggtgcaaatattaataatattaagccggcaagc | |
| | tttgatttgataaatataccaatcccaccacttgccgaacaaaaaatcatcgctgaa | |
| | aaactcgatacgctgctggcgcaggtagacagcaccaaagcacgttttgagcaaatc | |
| | ccacaaatcctgaaacgttttcgtcaagcggtattggggggcgcagttaatggaaaa | |
| | ttgacagaaaaatggcgtaattttgagccgcaacattctgtatttaagaagttaaat | |
| | tttgaatctatcttaactgaattacgtaatggtctttcatcaaagccaaatgaaagt | |
| | ggtgttggtcatccaatactacgcattagttctgtacgtgctggccatgtagatcaa | |
| | aacgatattcggtttctagaatgttcagaaagtgaactaaaccgccacaaattacaa | |
| | gatggagatcttttatttactcgctataacggaagtttagaatttgttggtgtttgt | |
| | gggttattgaaaaaattacaacatcaaaatttgctatatcctgataaacttattcga | |
| | gctcgattaaccaaagatgctttaccagaatatatcgaaatatttttttcatccccc | |
| | tcagcacgaaatgcaatgatgaactgcgtgaaaacaacttctggtcaaaaaggtatt | |
| | tcaggaaaagatatcaaatcccaagttgttttattacctccagtaaaagaacaagcc | |
| | gaaatcgttcgccgcgtcgagcaactcttcgcctacgccgacaccatagaaaaacag | |
| | gtcaacaacgccttagcccgcgtcaacaacctgacgcaatccatcctggcaaaagcg | |
| | ttccgtggtgaacttaccgcccagtggcgggccgaaaacccggatttgatcagcgga | |
| | gaaaacagcgccgccgcgttgctggaaaaaatcaaagctgaacgcgcagctagcggg | |
| | ggtaaaaaagcctcacgtaaaaaatcctgaacattattttctggcgcacctttccgg | |
| | tgcgctttttattatttcacgccaatcataacccacataaatatatttaaatcattc | |
| | cagaaattgcccattttattctatttttagctggactttccccatatttactgatga | |
| | tatatacaggtatttagcgcggtgcggatgtgcgccaacacacccgcatcgctaatc | |
| | acaatcactattcctggagaatagcagttatgactgacacgcattctattgcacaac | |
| | cgttcgaagcagaagtctccccggcaaataaccgtcatgtcaccgtcggttatgcga | |
| | gtcgctacccggattacagccgtattcccgccatcaccctgaaaggtcagtggctgg | |
| | aagccgccggttttgccactggcacggcggtagatgtcaaagtgatggaaggctgta | |
| | ttgtcctcaccgcccaaccacccgccgccgaggagagcgaactgatgcagtcgctac | |
| | gccaggtgtgcaaactgtcggcgcgtaaacaaaagcaggtgcaggcgtttattggag | |
| | tgattgccggtaaacagaaagtcgcgtaacagcgtttactcccggttaaccaccggg | |
| | agccttccactgactcaatagaaactttccccctcagtaaatatttaccagtctgat | |
| | tttgcagtaaaaatctattgtttcagtacgttgcgaaagcgataatagaggcttagc | |
| | aatgaggaaggcatatcttatggaatctattcaaccctggattgaaaaatttattaa | |
| | gcaagcacagcaacaacgttcgcaatccactaaagattatccaacgtcttaccgtaa | |
| | cctgcgagtaaaattgagtttcggttatggtaattttacgtctattccctggtttgc | |
| | atttcttggagaaggtcaggaagcttctaacggtatatatcccgttattctctatta | |
| | taaagattttgatgagttggttttggcttatggtataagcgacacgaatgaaccaca | |
| | tgcccaatggcagttctcttcagacatacctaaaacaatcgcagagtattttcaggc | |
| | aacttcgggtgtatatcctaaaaaatacggacagtcctattacgcctgttcccaaaa | |
| | agtctcacagggtattgattacacccgatttgcctctatgctggacaacataatcaa | |
| | cgactataaattaatatttaattctggcaagagtgttattccacctatgtcaaaaac | |
| | tgaatcatactgtctggaagatgcgttaaatgatttgtttatccctgaaaccacaat | |
| | agagacgatactcaaacgattaaccatcaaaaaaaatattatcctccaggggccgcc | |
| | cggcgttggaaaaacctttgttgcacgccgtctggcttacttgctgacaggagaaaa | |
| | ggctccgcaacgcgtcaatatggttcagttccatcaatcttatagctatgaggattt | |
| | tatacagggctatcgtccgaatggcgtcggcttccgacgtaaagacggcatatttta | |
| | caatttttgtcagcaagctaaagagcagccagagaaaaagtatatttttattataga | |
| | tgaaatcaatcgtgccaatctcagtaaagtatttggcgaagtgatgatgttaatgga | |
| | acatgataaacgaggtgaaaactggtctgttcccctaacctactccgaaaacgatga | |
| | agaacgattctatgtcccggagaatgtttatatcatcggtttaatgaatactgccga | |
| | tcgctctctggccgttgttgactatgccctacgcagacgattttctttcatagatat | |
| | tgagccaggttttgatacaccacagttccggaattttttactgaataaaaaagcaga | |
| | accttcatttgttgagtctttatgccaaaaaatgaacgagttgaaccaggaaatcag | |
| | caaagaggccactatccttgggaaaggattccgcattgggcatagttacttctgctg | |
| | tgggttggaagatggcacctctccggatacgcaatggcttaatgaaattgtgatgac | |
| | ggatatcgcccctttactcgaagaatatttctttgatgacccctataaacaacagaa | |
| | atggaccaacaaattattaggggactcatagtggaacagcccgtgatacctgtccgt | |
| | aatatctattacatgcttacctatgcatggggttatttacaggaaattaagcaggca | |
| | aaccttgaagccatacccggtaacaatcttcttgatatcctggggtatgtattaaat | |
| | aaaggggttttacagctttcacgccgagggcttgagcttgattacaatcctaacacc | |
| | gagatcattcctggcatcaaagggcgaatagagtttgctaaaacaatacgcggcttc | |
| | catcttaatcatgggaaaaccgtcagtacttttgatatgcttaatgaagacacgctg | |
| | gctaaccgaattataaaaagcacattagccatattaattaagcatgaaaagttaaat | |
| | tcaactatcagagatgaagctcgttcactttatagaaaattaccgggcattagcact | |
| | cttcatttaactccgcagcatttcagctatctgaatggcggaaaaaatacgcgttat | |
| | tataaattcgttatcagtgtctgcaaattcatcgtcaataattctattccaggtcaa | |
| | aacaaaggacactaccgtttctatgattttgaaagaaacgaaaaagagatgtcatta | |
| | ctttatcaaaagtttctttatgaattttgccgtcgtgaattaacgtctgcaaacaca | |
| | acccgctcttatttaaaatgggatgcatcgagtatatcggatcagtcacttaatttg | |
| | ttacctcgaatggaaactgacatcaccattcgctcatcagaaaaaatacttatcgtt | |
| | gacgccaaatactataagagcattttttcacgacgaatgggaacagaaaaatttcat | |
| | tcgcaaaatctttatcaactgatgaattacttatggtcgttaaagcctgaaaatggc | |
| | gaaaacataggggggttattaatatatccccacgtagataccgcagtgaaacatcgt | |
| | tataaaattaatggcttcgatattggcttgtgtaccgtcaatttaggtcaggaatgg | |
| | ccgtgtatacatcaagaattactcgacattttcgatgaatatctcaaataaaatatc | |
| | aggccggatgcggctgcgccttatccggcccataaccccttacttcctcaaccccgc | |
| | aaacgcagcccgaatctcttcctccggcagctggatc | |
| 48 | caatttctacaaaacacttgatactgtatgagcatacagtataattgcttcaacaga | Mrr-hsdRMS- |
| | acatattgactatccggtattacccggcatgacaggagtaaaaatggctatcgacga | symRE-mcrBC |
| | aaacaaacagaaagcgttggcggcagcactgggccagattgagaaacaatttggtaa | locus |
| | aggctccatcatgcgcctgggtgaagaccgttccatggatgtggaaaccatctctac | (deletion) |
| | cggttcgctttcactggatatcgcgcttggggcagqtggtctgccgatgggccgtat | (sequence |
| | cgtcgaaatctacggaccggaatcttccggtaaaaccacgctgacgctgcaggtgat | upstream: |
| | cgccgcagcgcagcgtgaaggtaaaacctgtgcgtttatcgatgctgaacacgcgct | single |
| | ggaccaatctacgcacgtaaactgggcgtcgatatcgacaacctgctgtgctccca | underline; |
| | gccggacaccggcgagcaggcactggaaatctgtgacgccctggcgcgttctggcgc | downstream of |
| | agtagacgttatcgtcgttgactccgtggcggcactgacgccgaaagcggaaatcga | ORF: italics; |
| | aggcgaaatcggcgactctcacatgggccttgcggcacgtatgatgagccaggcgat | and ORF in |
| | gcgtaagctggcgggtaacctgaagcagtccaacacgctgctgatcttcatcaacca | double |
| | gatccgtatgaaaattggtgtgatcttcggtaacccggaaaccactaccggtggtaa | underline) |
| | cgcgctgaaattctacgcctctgttcgtctcgacatccgtcgtatcggcgcggtgaa | |
| | agagggcgaaaacgtggtgggtagcgaaacccgcgtgaaagtggtgaagaacaaaat | |
| | cgctgcgccgtttaaacaggctgaattccagatcctctacggcgaaggtatcaactt | |
| | ctacggcgaactggttgacctgggcgtaaaagagaagctgatcgagaaagcaggcgc | |
| | gtggtacagctacaaaggtgagaagatcggtcagggtaaagcgaatgcgactgcctg | |
| | gctgaaagataacccggaaaccgcgaaagagatcgagaagaaagtacgtgagttgct | |
| | gctgagcaacccgaactcaacgccggatttctctgtagatgatagcgaaggcgtagc | |
| | agaaactaacgaagatttttaatcgtcttgtttgatacacaagggtcgcatctgcgg | |
| | cccttttgcttttttaagttgtaaggatatgccatgacagaatcaacatcccgtcgc | |
| | ccggcata | |

*Unless otherwise specified, sequences are depicted and listed, and are to be read:-5′-to-3′ for nucleotide sequences; and -N-terminus to C-terminus for amino acid sequences.
** Unless otherwise specified, NT denotes nucleotide sequences and AA denotes amino acid sequences

### Equivalents and Scope

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list. “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims. “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of,” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A genetically modified microorganism comprising a genome in which an ompT gene and a lon gene have been mutated, disabled, or deleted.

2. The genetically modified microorganism of claim 1, wherein the microorganism does not express a functional form of one or more proteins selected from the group consisting of G6PI, ManA, ManB, ManC, Gmd, Fcl, and KduI.

3. The genetically modified microorganism of claim 1 or 2, wherein the genome comprises a mutation in a gene selected from the group consisting of pgi, manA, manB, manC, gmd, fcl, and kduI.

4. The genetically modified microorganism of any one of claims 1-3, wherein the genome comprises a mutation in a promoter operably linked to a gene selected from the group consisting of pgi, manA, manB, manC, gmd, fcl, and kduI.

5. The genetically modified microorganism of any one of claims 1-4, wherein the genome does not comprise a nucleic acid sequence encoding a carbohydrate metabolism protein selected from the group consisting of G6PI, ManA, ManB, ManC, Gmd, Fcl, and KduI.

6. The genetically modified microorganism of any one of claims 1-5, wherein the microorganism does not express functional ManA.

7. The genetically modified microorganism of any one of claims 1-6, wherein the genome comprises a mutation in a manA gene or a promoter operably linked to the manA gene.

8. The genetically modified microorganism of any one of claims 1-7, wherein the genome does not comprise a nucleic acid sequence encoding ManA.

9. The genetically modified microorganism of any one of claims 1-8, wherein the genome comprises i) a first nucleic acid sequence having at least 90% sequence identity to SEQ ID NO: 23, and ii) a second nucleic acid sequence having at least 90% sequence identity to SEQ ID NO: 25.

10. The genetically modified microorganism of claim 9, wherein the genome further comprises a third nucleic acid sequence having at least 90% sequence identity to SEQ ID NO: 24.

11. The genetically modified microorganism of any one of claims 1-10, wherein the microorganism does not exhibit a mucoid phenotype.

12. The genetically modified microorganism of any one of claims 1-11, wherein the microorganism is not capable of synthesizing mannose.

13. The genetically modified microorganism of any one of claims 1-12, wherein the microorganism is not capable of synthesizing fucose.

14. The genetically modified microorganism of any one of claims 1-13, wherein
i) the genome does not comprise a nucleic acid sequence encoding endA;
ii) the genome does not comprise a nucleic acid sequence encoding recA; and
iii) the EcoK1 restriction system has been inactivated.

15. The genetically modified microorganism of any one of claims 1-14, wherein the genotype of the microorganism is ΔendA ΔrecA Δ(mrr-hsdRMS-symE-mcrBC) ΔompT ΔmanA Δlon.

16. The genetically modified microorganism of any one of claims 1-15, wherein the microorganism is E. coli.

17. The genetically modified microorganism of claim 16, wherein the microorganism is derived from E. coli MG1655.

18. The genetically modified microorganism of any one of claims 1-17, further comprising a nucleic acid sequence encoding a recombinant protein.

19. The genetically modified microorganism of claim 18, wherein the nucleic acid sequence encoding a recombinant protein is located in the genome of the microorganism.

20. The genetically modified microorganism of claim 19, wherein the nucleic acid sequence encoding a recombinant protein is located on a plasmid.

21. The genetically modified microorganism of any one of claims 18-20, wherein the recombinant protein is an RNA polymerase.

22. The genetically modified microorganism of claim 21, wherein the RNA polymerase is a T7 RNA polymerase, T3 RNA polymerase, SP6 RNA polymerase, or K11 RNA polymerase.

23. The genetically modified microorganism of claim 22, wherein the RNA polymerase comprises
(a) an amino acid substitution at a binding site residue for de novo RNA synthesis; and
(b) an amino acid modification that causes increased transcription efficiency, relative to wild-type RNA polymerase.

24. The genetically modified microorganism of claim 23, wherein the amino acid modification is an amino acid substitution at position 47, relative to the wild-type RNA polymerase, wherein the wild-type RNA polymerase comprises the amino acid sequence of SEQ ID NO: 34.

25. The genetically modified microorganism of claim 24, wherein the amino acid substitution at position 47 is G47A.

26. The genetically modified microorganism of any one of claims 23-25, wherein the amino acid modification comprises an additional C-terminal amino acid, relative to the wild-type RNA polymerase.

27. The genetically modified microorganism of claim 26, wherein the additional C-terminal amino acid is glycine.

28. The genetically modified microorganism of any one of claims 23-27, wherein the RNA polymerase comprises the amino acid sequence of SEQ ID NO: 41.

29. The genetically modified microorganism of any one of claims 21-27, wherein the RNA polymerase comprises
(a) an amino acid substitution at position 350; and/or
(b) an amino acid substitution at position 351
relative to the wild-type RNA polymerase, wherein the wild-type RNA polymerase comprises the amino acid sequence of SEQ ID NO: 34.

30. The genetically modified microorganism of claim 29, wherein the amino acid substitution at position 350 is E350W.

31. The genetically modified microorganism of claim 29, wherein the amino acid substitution at position 351 is D351V.

32. The genetically modified microorganism of claim 29, wherein the amino acid substitution at position 350 is E350W, and the amino acid substitution at position 351 is D351V.

33. The genetically modified microorganism of any one of claims 29-32, wherein the RNA polymerase comprises the amino acid sequence of SEQ ID NO: 42.

34. The genetically modified microorganism of any one of claim 23 or 29-32, wherein the RNA polymerase comprises the amino acid sequence of SEQ ID NO: 43.

35. A method for producing a polypeptide or protein, comprising the steps of
i) introducing a nucleic acid molecule comprising a sequence encoding a polypeptide or protein into the genetically modified microorganism of any one of claims 1-17;
ii) culturing the genetically modified organism under conditions suitable for expression of the polypeptide or protein; and
iii) isolating the polypeptide or protein.

36. A method for producing a polypeptide or protein, comprising the steps of
i) culturing the genetically modified organism of any one of claims 18-34 under conditions suitable for expression of the polypeptide or protein; and
ii) isolating the polypeptide or protein.

37. The method of claim 35 or 36, wherein the polypeptide or protein is an RNA polymerase.

38. The method of claim 37, wherein the RNA polymerase is a T7 RNA polymerase, T3 RNA polymerase, SP6 RNA polymerase, or K11 RNA polymerase.

39. The method of claim 38, wherein the RNA polymerase comprises
(a) an amino acid substitution at a binding site residue for de novo RNA synthesis; and
(b) an amino acid modification that causes increased transcription efficiency, relative to a wild-type RNA polymerase.

40. The method of claim 39, wherein the amino acid modification is an amino acid substitution at position 47, relative to the wild-type RNA polymerase, wherein the wild-type RNA polymerase comprises the amino acid sequence of SEQ ID NO: 34.

41. The method of claim 40, wherein the amino acid substitution at position 47 is G47A.

42. The method of any one of claims 39-41, wherein the amino acid modification comprises an additional C-terminal amino acid, relative to the wild-type RNA polymerase.

43. The method of claim 42, wherein the additional C-terminal amino acid is glycine.

44. The method of any one of claims 40-43, wherein the RNA polymerase comprises the amino acid sequence of SEQ ID NO: 41.

45. The method of any one of claims 37-43, wherein the RNA polymerase comprises
(a) an amino acid substitution at position 350; and
(b) an amino acid substitution at position 351
relative to the wild-type RNA polymerase, wherein the wild-type RNA polymerase comprises the amino acid sequence of SEQ ID NO: 34.

46. The method of claim 45, wherein the amino acid substitution at position 350 is E350W.

47. The method of claim 45, wherein the amino acid substitution at position 351 is D351V.

48. The method of claim 45, wherein the amino acid substitution at position 350 is E350W, and the amino acid substitution at position 351 is D351V.

49. The method of any one of claims 45-48, wherein the RNA polymerase comprises the amino acid sequence of SEQ ID NO: 42.

50. The method of any one of claim 39 or 45-48, wherein the RNA polymerase comprises the amino acid sequence of SEQ ID NO: 43.
